SEQUENCE LISTING (26 sequences)

SEQ ID NO: 1 - protein, 368 aa, Homo sapiens
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 2 - DNA, 2593 bp, Homo sapiens
  source 1..2593: mol_type = genomic DNA
acaagcaccaaagcagaggggcaggcagcacaccacccagcagccagagcaccagcccagccatggtccttgaggtaagtgctgctgcccagccaggccaggccagaattcgaagtggaaaacccggggcatcaggggaatgccagggctggcccacagcccagggtcaccacagggttgggtgagtagctggaggccgcatgggttgtggcagaaacagcactggggaattgggaacccagcttggccactgaccctgggtgcctctcctctcctctctggcttcggtgtctcgtctgcaaaatggggggggcggggggcaagtagactcgaaggctcccttcaggagacttggaactttgactcttggcgtgctggactaaattggtactggggagacctgcaggtccatagtgctgggggtgaggggataaagaagattccttctcaagctaagtgggggatccccttgtgggcagctaaatggccgtgcaagcctcagtgtagggagggggaaggactgggcagactgcatgcatttggggggcgtggtgacttgtagagaggccacacggtgggggggggcgcaggaagttggggtccccggacaagggccaaggagggaagaggagggttagtgactggaaaaggggattgtggagaggcgctggggaggaaggtggttgaactatgtgttgggggtggggcactgaggacgcaaggcaagcctgaagggagagcagggagagagaggacagtgggcagagagggctctgggcactggagggacgctcttcttcctgcccaggggtccctgggccgatgggatcacgcagaagaatgcgagagaagcagcctttgagaagggaagtcactatcccagagcccaggctgagcggatggagttgaggaagtacggccctggaagactggcggggacagttataggaggagctgctcagagtaaatcacagactaaatcagactcaatcacaaaagagttcctgccaggcctttacacagccccttcctccccgttcccgccctcacaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggtgggccgcacggctctgcgggtgctgcagctggtggctggctttctgctgcccctgctggtcatggcctactgctatgcccacatcctggccgtgctgctggtttccaggggccagcggcgcctgcgggccatgcggctggtggtggtggtcgtggtggcctttgccctctgctggaccccctatcacctggtggtgctggtggacatcctcatggacctgggcgctttggcccgcaactgtggccgagaaagcagggtagacgtggccaagtcggtcacctcaggcctgggctacatgcactgctgcctcaacccgctgctctatgcctttgtaggggtcaagttccgggagcggatgtggatgctgctcttgcgcctgggctgccccaaccagagagggctccagaggcagccatcgtcttcccgccgggattcatcctggtctgagacctcagaggcctcctactcgggcttgtgaggccggaatccgggctcccctttcgcccacagtctgacttccccgcattccaggctcctccctccctctgccggctctggctctccccaatatcctcgctcccgggactcactggcagccccagcaccaccaggtctcccgggaagccaccctcccagctctgaggactgcaccattgctgctccttagctgccaagccccatcctgccgcccgaggtggctgcctggagccccactgcccttctcatttggaaactaaaacttcatcttccccaagtgcggggagtacaaggcatggcgtagagggtgctgccccatgaagccacagcccaggcctccagctcagcagtgactgtggccatggtccccaagacctctatatttgctcttttatttttatgtctaaaatcctgcttaaaacttttcaataaacaagatcgtcagga

SEQ ID NO: 3 - DNA, 720 bp, Homo sapiens
  source 1..720: mol_type = genomic DNA
atggtccttgaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggtgggccgcacggctctgcgggtgctgcagctggtggctggctttctgctgcccctgctggtcatggcctactgctatgcccacatcctggcc

SEQ ID NO: 4 - DNA, 369 bp, synthetic construct
  source 1..369: mol_type = other DNA
cagcagcagctggaggagtccgggggaggcctggtcaagcctgagggatccctgacactcacctgcaaagcctctggattcgacctcggtttctacttttacgcctgttgggtccgccaggctccagggaagggcctggagtggatcgcatgcatttatactgctggtagtggtagcacgtactacgcgagctgggcgaaaggccgattcaccatctccaaagcctcgtcgaccacggtgactctgcaaatgaccagtctggcagccgcggacacggccacctatttctgtgcgagatctactgctaatactagaagtacttattatcttaacttgtggggcccaggcaccctggtcaccgtctcctca

SEQ ID NO: 5 - protein, 123 aa, synthetic construct
QQQLEESGGGLVKPEGSLTLTCKASGFDLGFYFYACWVRQAPGKGLEWIACIYTAGSGSTYYASWAKGRFTISKASSTTVTLQMTSLAAADTATYFCARSTANTRSTYYLNLWGPGTLVTVSS

SEQ ID NO: 6 - DNA, 330 bp, synthetic construct
  source 1..330: mol_type = other DNA
gacgtcgtgatgacccagactccagcctccgtgtctgaacctgtgggaggcacagtcaccatcaagtgccaggccagtcagaggattagtagttacttatcctggtatcagcagaaaccagggcagcgtcccaagctcctgatctttggtgcatccactctggcatctggggtcccctcgcggttcaaaggcagtggatctgggacagaatacactctcaccatcagcgacctggagtgtgccgatgctgccacttactactgtcagagttatgcttattttgatagtaataattggcatgctttcggcggagggaccgaggtggtggtc

SEQ ID NO: 7 - protein, 110 aa, synthetic construct
DVVMTQTPASVSEPVGGTVTIKCQASQRISSYLSWYQQKPGQRPKLLIFGASTLASGVPSRFKGSGSGTEYTLTISDLECADAATYYCQSYAYFDSNNWHAFGGGTEVVV

SEQ ID NO: 8 - DNA, 5872 bp, Homo sapiens
  source 1..5872: mol_type = genomic DNA
gcagaagcctgaagaccaaggagtggaaagttctccggcagccctgagatctcaagagtgacatttgtgagaccagctaatttgattaaaattctcttggaatcagctttgctagtatcatacctgtgccagatttcatcatgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgtgaaatggaagtcaatagggctgttgggactttcttgaaaagaagcaaggaaatatgagtcatccgctatcacagctttcaaaagcaagaacaccatcctacataatacccaggattcccccaacacacgttcttttctaaatgccaatgagttggcctttaaaaatgcaccactttttttttttttttgacagggtctcactctgtcacccaggctggagtgcagtggcaccaccatggctctctgcagccttgacctctgggagctcaagtgatcctcctgcctcagtctcctgagtagctggaactacaaggaagggccaccacacctgactaacttttttgttttttgtttggtaaagatggcatttcaccatgttgtacaggctggtctcaaactcctaggttcactttggcctcccaaagtgctgggattacagacatgaactgccaggcccggccaaaataatgcaccacttttaacagaacagacagatgaggacagagctggtgataaaaaaaaaaaaaaaaaagcattttctagataccacttaacaggtttgagctagtttttttgaaatccaaagaaaattatagtttaaattcaattacatagtccagtggtccaactataattataatcaaaatcaatgcaggtttgttttttggtgctaatatgacatatgacaataagccacgaggtgcagtaagtacccgactaaagtttccgtgggttctgtcatgtaacacgacatgctccaccgtcaggggggagtatgagcagagtgcctgagtttagggtcaaggacaaaaaacctcaggcctggaggaagttttggaaagagttcaagtgtctgtatatcctatggtcttctccatcctcacaccttctgcctttgtcctgctcccttttaagccaggttacattctaaaaattcttaacttttaacataatattttataccaaagccaataaatgaactgcatatgataggtatgaagtacagtgagaaaattaacacctgtgagctcattgtcctaccacagcactagagtgggggccgccaaactcccatggccaaacctggtgcaccatttgcctttgtttgtctgttggtttgcttgagacagtcttgctctgttgcccaggctggaatggagtggctattcacaggcacaatcatagcacactttagccttaaactcctgggctcaagtgatccacccgcctcagtctcccaagtagctgggattacaggtgcaaacctggcatgcctgccattgtttggcttatgatctaaggatagctttttaaattttattcattttatttttttttgagacagtgtctcactctgtctcccaggctggagtacagtggtacaatcttggatcaccgcctcccagtttcaagtgatctccctgcctcagcctcctaagtagctgggactacaggtatgtgccaccacgcctggctaatttttatatttttagtagagacggggtttcaccatgttgtccaggctggtctcaaactcctgacctcaggtgatctgcccacctctgcctcccaaagtgctgggattacaggcatgagccaccatgcctggccatttcttacacttttgtatgacatgcctattgcaagcttgcgtgcctctgtcccatgttattttactctgggatttaggtggagggagcagcttctatttggaacattggccatcgcatggcaaatgggtatctgtcacttctgctcctatttagttggttctactataacctttagagcaaatcctgcagccaagccaggcatcaatagggcagaaaagtatattctgtaaataggggtgaggagaagatatttctgaacaatagtctactgcagtaccaaattgcttttcaaagtggctgttctaatgtactcccgtcagtcatataagtgtcatgtaagtatcccattgatccacatccttgctaccctctggtactatcaggtgcccttaattttgccaagccagtgggtatagaatgagatctcactgtggtcttagtttgcatttgcttggttactgatgagcaccttgtcaaatatttatataccatttgtgtttatttttttaaataaaatgcttgctcatgcttttttgcccatttgcaaaaaaacttggggccgggtgcagtggctcatgcctgtagtcccagctctttgggaggccaaggtgggcagatcgcttgagcccaggagttcgagaccagccttggcaacatggcgaaaccctgtctttacaaaaaatacaaaaattagccgggtgtggtggtgtgcacctgaagtcccagctactcagtaggttcgctttgagcctgggaggcagaggttgcagtgagctgggaccgcatcactacacttcagcctgggcaacagagaaaaaccttttctcagaaacaaacaaacccaaatgtggttgtttgtcctgattcctaaaaggtctttatgtattctagataataatctttggtcagttatatgtgttaaaaaatatcttctttgtggccaggcacggtagctcacacctgtaatcccagcactttgcggggctgaggtgggtggatcatctgaggtcaagagttcaagatcagcctggccaacacagtgaaaccccatctctactaaacatgtacaaaacttagctgggtatggtggcgggtgcctgtaaccccagctgctccagaggctgtggcagaagaatcgcttgaacccaggaggcagaggttgcagcgagccaagattgtgccattgcactccagactgggtgacaagagtgaaattctgcctatctatctatctatctatctatatctatatatatatatatatatatcctttgtaatttatttttccctttttaaaattttttataaaattcttttttatttttatttttagcagaggtgaggtttctgaggtttcattatgttgcccaggctggtcttgaactcctgagctcaagtgatcctcccacctcagccttccaaagtgctggaattgcagacatgagccaccgcgcccctcctgtttttctctaattaatggtgtctttctttgtctttctggtaataagcaaaaagttcttcatttgatttggttaaatttataactgttttctcatatggttaacattttttcttgcctggctaaagaaatccttttctgcccaatactataaagaggtttgcccacattttattccaaaagttttaagttttgtctttcatcttgaagtctaatgtatcaggaactggcttttgtgcctgttgggaggtagtgatccaattccatgtcttgcatgtaggtaaccactggtccctgcgccatgtattcaatacgtcgtctttctcctgcgggtctgcaatctcacctaccatccatcaagtttccatagggccatgggtctgcttctgggctccctgttctgttccattgtcaatttgtctatcctgtgccagtatcacactgtgtttattacaatagctttgtaacagctctcgatatccggtaggacatctccctccaccttctttttctacttcagaagtgtcttagctaggtcaggcacggtggctcacgcctgtaatcccagcactttgggaggccgacgcggatggatcacctgaggtcaggagttttgagacagcctggccaacatggtgaaaccccatctctactaaaaaatacaaaaattagtcaggcatggtggcatgtgcctgtaatcccagctatttgggaggctgaggccggagaattgcttgaacccggggggcggaggttgcagtgagccgagatcgtaccattgcactccagcctgggtgacagagcgaaactctgtctcaggaaaaaaaagaaaagagatgtcttggttattcttggttctttattattcaatataaattttagaagctgaatttgaaaagatttggattggaatttcattaaatctacaggtcaatttagggagagttgataattttacagaattgagtcatctggtgttccaataagaataagagaacaattattggctgtacaattcttgccaaatagtaggcaaagcaaagcttaggaagtatactggtgccatttcaggaacaaagctaggtgcgaatatttttgtctttctgaatcatgatgctgtaagttctaaagtgatttctcctcttggctttggacacatggtgtttaattacctactgctgactatccacaaacagaaagagactggtcatgccccacagggttggggtatccaagataatggagcgaggctctcatgtgtcctaggttacacaccgaaaatccacagtttattctgtgaagaaaggaggctatgtttatgatacagactgtgatatttttatcatagcctattctggtatcatgtgcaaaagctataaatgaaaaacacaggaacttggcatgtgagtcattgctccccctaaatgacaattaataaggaaggaacattgagacagaataaaatgatccccttctgggtttaatttagaaagttccataattaggtttaatagaaataaatgtaaatttctatgattaaaaataaattagcacatttagggatacacaaattataaatcattttctaaatgctaaaaacaagctcaggtttttttcagaagaaagttttaattttttttctttagtggaagatatcactctgacggaaagttttgatgtgaggggcggatgactataaagtgggcatcttcccccacaggaagatgtttccatctgtgggtgagaggtgcccaccgcagctagggcaggttacatgtgccctgtgtgtggtaggacttggagagtgatctttatcaacgtttttatttaaaagactatctaataaaacacaaaactatgatgttcacaggaaaaaaagaataagaaaaaaaga

SEQ ID NO: 9 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 10 - DNA, 1597 bp, Homo sapiens
  source 1..1597: mol_type = genomic DNA
aaccgtcccggccaccgctgcctcagcctctgcctcccagcctctttctgagggaaaggacaagatgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacagccaggggatttcaccactcaaaggccagacctgcagacgcccagattatgagacacaggatgaagcatttacaacccggttcactcttctcagccactgaagtattcccctttatgtacaggatgctttggttatatttagctccaaaccttcacacacagactgttgtccctgcactctttaagggagtgtactcccagggcttacggccctggccttgggccctctggtttgccggtggtgcaggtagacctgtctcctggcggttcctcgttctccctgggaggcgggcgcactgcctctcacagctgagttgttgagtctgttttgtaaagtccccagagaaagcgcagatgctagcacatgccctaatgtctgtatcactctgtgtctgagtggcttcactcctgctgtaaatttggcttctgttgtcaccttcacctcctttcaaggtaactgtactgggccatgttgtgcctccctggtgagagggccgggcagaggggcagatggaaaggagcctaggccaggtgcaaccagggagctgcaggggcatgggaaggtgggcgggcaggggagggtcagccagggcctgcgagggcagcgggagcctccctgcctcaggcctctgtgccgcaccattgaactgtaccatgtgctacaggggccagaagatgaacagactgaccttgatgagctgtgcacaaagtggcataaaaaacatgtggttacacagtgtgaataaagtgctgcggagcaagaggaggccgttgattcacttcacgctttcagcgaatgacaaaatcatctttgtgaaggcctcgcaggaagacccaacacatgggacctataactgcccagcggacagtggcaggacaggaaaaacccgtcaatgtactaggatactgctgcgtcattacagggcacaggccatggatggaaaacgctctctactctgctttttttctactgttttaatttatactggcatgctaaagccttcctattttgcataataaatgcttcagtgaaaatgca

SEQ ID NO: 11 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 12 - DNA, 66 bp, porcine teschovirus-1
  source 1..66: mol_type = unassigned DNA
ggcagcggcgccacaaacttctctctgctaaagcaagcaggtgatgttgaagaaaaccccgggcct

SEQ ID NO: 13 - protein, 22 aa, porcine teschovirus-1
GSGATNFSLLKQAGDVEENPGP

SEQ ID NO: 14 - DNA, 63 bp, thosea asigna virus
  source 1..63: mol_type = unassigned DNA
ggctccggcgagggcaggggaagtcttctacactgcggggacgtggaggaaaatcccggccca

SEQ ID NO: 15 - protein, 21 aa, thosea asigna virus
GSGEGRGSLLTCGDVEENPGP

SEQ ID NO: 16 - DNA, 69 bp, equine rhinitis A virus
  source 1..69: mol_type = unassigned DNA
ggctcgggccagtgtactaattatgctctcttgaaattggctggagatgttgagagcaacccaggtccc

SEQ ID NO: 17 - protein, 23 aa, equine rhinitis A virus
GSGQCTNYALLKLAGDVESNPGP

SEQ ID NO: 18 - DNA, 720 bp, synthetic construct
  source 1..720: mol_type = other DNA
ttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccat

SEQ ID NO: 19 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 20 - DNA, 8657 bp, synthetic construct
  source 1..8657: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagcggccgctgagttaactattctagatggccctccctgtcaccgccctgctgcttccgctggctcttctgctccacgccgctcggccctacccatacgacgttccagactacgctgctagcgacgtcgtgatgacccagactccagcctccgtgtctgaacctgtgggaggcacagtcaccatcaagtgccaggccagtcagaggattagtagttacttatcctggtatcagcagaaaccagggcagcgtcccaagctcctgatctttggtgcatccactctggcatctggggtcccctcgcggttcaaaggcagtggatctgggacagaatacactctcaccatcagcgacctggagtgtgccgatgctgccacttactactgtcagagttatgcttattttgatagtaataattggcatgctttcggcggagggaccgaggtggtggtcggtggaggtggcagcggaggaggtgggtccggcggtggaggaagccagcagcagctggaggagtccgggggaggcctggtcaagcctgagggatccctgacactcacctgcaaagcctctggattcgacctcggtttctacttttacgcctgttgggtccgccaggctccagggaagggcctggagtggatcgcatgcatttatactgctggtagtggtagcacgtactacgcgagctgggcgaaaggccgattcaccatctccaaagcctcgtcgaccacggtgactctgcaaatgaccagtctggcagccgcggacacggccacctatttctgtgcgagatctactgctaatactagaagtacttattatcttaacttgtggggcccaggcaccctggtcaccgtctcctcaaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggggatccggcgcaacaaacttctctctgctgaaacaagccggagatgtcgaagagaatcctggaccgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgagctagcctcgagatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 21 - DNA, 9058 bp, synthetic construct
  source 1..9058: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagcggccgctgagttaactattctagatggccctccctgtcaccgccctgctgcttccgctggctcttctgctccacgccgctcggccctacccatacgacgttccagactacgctgctagcgacgtcgtgatgacccagactccagcctccgtgtctgaacctgtgggaggcacagtcaccatcaagtgccaggccagtcagaggattagtagttacttatcctggtatcagcagaaaccagggcagcgtcccaagctcctgatctttggtgcatccactctggcatctggggtcccctcgcggttcaaaggcagtggatctgggacagaatacactctcaccatcagcgacctggagtgtgccgatgctgccacttactactgtcagagttatgcttattttgatagtaataattggcatgctttcggcggagggaccgaggtggtggtcggtggaggtggcagcggaggaggtgggtccggcggtggaggaagccagcagcagctggaggagtccgggggaggcctggtcaagcctgagggatccctgacactcacctgcaaagcctctggattcgacctcggtttctacttttacgcctgttgggtccgccaggctccagggaagggcctggagtggatcgcatgcatttatactgctggtagtggtagcacgtactacgcgagctgggcgaaaggccgattcaccatctccaaagcctcgtcgaccacggtgactctgcaaatgaccagtctggcagccgcggacacggccacctatttctgtgcgagatctactgctaatactagaagtacttattatcttaacttgtggggcccaggcaccctggtcaccgtctcctcaaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggggatccggcgcaacaaacttctctctgctgaaacaagccggagatgtcgaagagaatcctggaccgatggtgctggaggtgagcgaccaccaggtgctgaacgacgccgaggtggccgccctgctggagaacttcagcagcagctacgactacggcgagaacgagagcgacagctgctgcaccagccccccctgcccccaggacttcagcctgaacttcgacagggccttcctgcccgccctgtacagcctgctgttcctgctgggcctgctgggcaacggcgccgtggccgccgtgctgctgagcaggaggaccgccctgagcagcaccgacaccttcctgctgcacctggccgtggccgacaccctgctggtgctgaccctgcccctgtgggccgtggacgccgccgtgcagtgggtgttcggcagcggcctgtgcaaggtggccggcgccctgttcaacatcaacttctacgccggcgccctgctgctggcctgcatcagcttcgacaggtacctgaacatcgtgcacgccacccagctgtacaggaggggcccccccgccagggtgaccctgacctgcctggccgtgtggggcctgtgcctgctgttcgccctgcccgacttcatcttcctgagcgcccaccacgacgagaggctgaacgccacccactgccagtacaacttcccccaggtgggcaggaccgccctgagggtgctgcagctggtggccggcttcctgctgcccctgctggtgatggcctactgctacgcccacatcctggccgtgctgctggtgagcaggggccagaggaggctgagggccatgaggctggtggtggtggtggtggtggccttcgccctgtgctggaccccctaccacctggtggtgctggtggacatcctgatggacctgggcgccctggccaggaactgcggcagggagagcagggtggacgtggccaagagcgtgaccagcggcctgggctacatgcactgctgcctgaaccccctgctgtacgccttcgtgggcgtgaagttcagggagaggatgtggatgctgctgctgaggctgggctgccccaaccagaggggcctgcagaggcagcccagcagcagcaggagggacagcagctggagcgagaccagcgaggccagctacagcggcctgtgagaattccctgcagggctagcctcgagatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 22 - DNA, 5927 bp, synthetic construct
  source 1..5927: mol_type = other DNA
ggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattctgacactatgggtttcactacgaaaattatctttctgtataatctggtactcgtatatgcgggtttcgacgatcccaggaaagcgatcgaacttgtccagaagagatacgggaggccctgtgactgcagcggagggcaagtatcagaacccccctctgatcgggtcagccaagttacttgcagcggcaaaacagcttacctgatgccggatcagagatggaaatgcaaatccatacccaaggacaccagtccgagtggaccattgcaggaatgtccgtgtaatagttaccaatcaagcgtccattcaagttgctacacgtcataccagcaatgtcgctcaggaaataaaacctattatacggcgacactgcttaaaacccaaacgggtggcacctctgatgttcaggttctcggaagtacgaataagttgattcagagtccctgcaacggtatcaaaggccagtcaatttgttggtctacgacagcgcctatccatgtgagtgacggcggtgggccgttggatacaacacgaataaaaagtgtacagcggaaacttgaggagatacacaaagccctctaccccgagcttcagtaccatcccctggccatccctaaggtcagggacaatctcatggtagacgctcaaaccctcaacatcctcaatgccacctacaatctcttgttgatgtctaacacaagcttggtagatgactgctggctctgtcttaaattgggccctccgactcccctcgctatacccaacttccttctgtcatacgtaacgcgcagctccgacaacatatcatgtctgataatcccgccgttgcttgtgcagcccatgcagttctctaacagctcctgcttgttcagtccatcttataattcaacagaagaaattgatttgggccatgtagctttcagtaactgtacatcaataactaacgtcactggccccatctgcgccgtgaacggttctgtcttcctctgcggcaacaatatggcttatacatacttgccaactaactggaccggtctgtgtgtattggccacgctgttgcctgacatagatataatccctggcgacgaacccgtccctatcccagccatcgaccattttatttatcgccccaagcgcgcgattcagtttatccctctgctcgctgggttgggcattacggctgcttttactacgggggctaccggccttggagtgtccgttacccaatatacgaaactgtccaatcaattgatttcagacgtgcaaatcttgagctctactatccaggatctgcaggaccaggtagactctctggcggaagtcgtcttgcaaaatcggcgggggttggatctgctgaccgccgagcagggcggcatctgtcttgctcttcaagaaaaatgctgtttttacgtgaacaaatcaggtattgtaagagataaaataaaaactttgcaagaagagctcgaaaggaggcggaaagacctggcgtctaatcctctgtggactggcctgcaggggctcctcccctatttgctgccctttcttggtccgctcctgactttgttgctgctcctgactattgggccatgcatcttcaatcgactcaccgcgttcatcaatgataaactcaacataatccacgctatgtgactcaaatcctgcacaacagattcttcatgtttggaccaaatcaacttgtgataccatgctcaaagaggcctcaattatatttgagtttttaatttttatgaaaaaaaaaaaaaaaaacggaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

SEQ ID NO: 23 - DNA, 12150 bp, synthetic construct
  source 1..12150: mol_type = other DNA
ttgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcgatcccgtaataagcttcgaggtccgcggccggccgcgttgacgcgcacggcaagaggcgaggggcggcgactggtgagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagctcgaggccgccccggtgaccttcagaccttggcactggaggtggcccggcagaagcgcggcatcgtggatcagtgctgcaccagcatctgctctctctaccaactggagaactactgcaactaggcccaccactaccctgtccacccctctgcaatgaataaaacctttgaaagagcactacaagttgtgtgtacatgcgtgcatgtgcatatgtggtgcggggggaacatgagtggggctggctggagtggcgatgataagctgtcaaacatgagaattaattcttgaagacgaaagggcctcgtgatacgcctatttttataggttaatgtcatgataataatggtttcttagtctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaacccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttgggctgcaggaattaattcgagctcgcccgaca

SEQ ID NO: 24 - DNA, 4392 bp, synthetic construct
  source 1..4392: mol_type = other DNA
gtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctacggcggcggcggagtttggccgtaggtggcgccctcttcctcccatgcgtgtgaccccgaagcccctcatcggctgaagcagggccaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagcccttgagtcaaagacgtagtcgttgcaagtccgcaccaggtactgatatcccaccaaaaagtgcggcggcggctggcggtagaggggccagcgtagggtggccggggctccgggggcgaggtcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatccgcggctgacgcggcggcagatggtgattacgaacccccgcggcgccgggcccggcactacctggacttggaggagggcgagggcctggcgcggctaggagcgccctctcctgagcgacacccaagggtgcagctgaagcgtgacacgcgcgaggcgtacgtgccgcggcagaacctgtttcgcgaccgcgagggagaggagcccgaggagatgcgggatcgaaagttccacgcagggcgcgagttgcggcatggcctgaaccgcgagcggttgctgcgcgaggaggactttgagcccgacgcgcggaccgggattagtcccgcgcgcgcacacgtggcggccgccgacctggtaaccgcgtacgagcagacggtgaaccaggagattaactttcaaaaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctaga

SEQ ID NO: 25 - DNA, 1641 bp, synthetic construct
  source 1..1641: mol_type = other DNA
atgggtttcactacgaaaattatctttctgtataatctggtactcgtatatgcgggtttcgacgatcccaggaaagcgatcgaacttgtccagaagagatacgggaggccctgtgactgcagcggagggcaagtatcagaacccccctctgatcgggtcagccaagttacttgcagcggcaaaacagcttacctgatgccggatcagagatggaaatgcaaatccatacccaaggacaccagtccgagtggaccattgcaggaatgtccgtgtaatagttaccaatcaagcgtccattcaagttgctacacgtcataccagcaatgtcgctcaggaaataaaacctattatacggcgacactgcttaaaacccaaacgggtggcacctctgatgttcaggttctcggaagtacgaataagttgattcagagtccctgcaacggtatcaaaggccagtcaatttgttggtctacgacagcgcctatccatgtgagtgacggcggtgggccgttggatacaacacgaataaaaagtgtacagcggaaacttgaggagatacacaaagccctctaccccgagcttcagtaccatcccctggccatccctaaggtcagggacaatctcatggtagacgctcaaaccctcaacatcctcaatgccacctacaatctcttgttgatgtctaacacaagcttggtagatgactgctggctctgtcttaaattgggccctccgactcccctcgctatacccaacttccttctgtcatacgtaacgcgcagctccgacaacatatcatgtctgataatcccgccgttgcttgtgcagcccatgcagttctctaacagctcctgcttgttcagtccatcttataattcaacagaagaaattgatttgggccatgtagctttcagtaactgtacatcaataactaacgtcactggccccatctgcgccgtgaacggttctgtcttcctctgcggcaacaatatggcttatacatacttgccaactaactggaccggtctgtgtgtattggccacgctgttgcctgacatagatataatccctggcgacgaacccgtccctatcccagccatcgaccattttatttatcgccccaagcgcgcgattcagtttatccctctgctcgctgggttgggcattacggctgcttttactacgggggctaccggccttggagtgtccgttacccaatatacgaaactgtccaatcaattgatttcagacgtgcaaatcttgagctctactatccaggatctgcaggaccaggtagactctctggcggaagtcgtcttgcaaaatcggcgggggttggatctgctgaccgccgagcagggcggcatctgtcttgctcttcaagaaaaatgctgtttttacgtgaacaaatcaggtattgtaagagataaaataaaaactttgcaagaagagctcgaaaggaggcggaaagacctggcgtctaatcctctgtggactggcctgcaggggctcctcccctatttgctgccctttcttggtccgctcctgactttgttgctgctcctgactattgggccatgcatcttcaatcgactcaccgcgttcatcaatgataaactcaacataatccacgctatgtga

SEQ ID NO: 26 - protein, 546 aa, synthetic construct
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAM

(19) United States
(12) Patent Application Publication
(10) Pub. No.: US 2026/0183396 A1
(43) Pub. Date: Jul. 2, 2026

(54) CXCR3 OVEREXPRESSION IN CAR-NK CELLS PRIMES MIGRATION/HOMING INTO THE TUMOR MICROENVIRONMENT

(71) Applicant: DANA-FARBER CANCER INSTITUTE, INC., Boston, MA (US)

(72) Inventors: Erik H. KNELSON, Boston, MA (US); Juan J. MIRET, Boston, MA (US); Marco CAMPISI, Boston, MA (US); David BARBIE, Boston, MA (US); Patrick H. LIZOTTE, Boston, MA (US); Mubin TARANNUM, Boston, MA (US); Rizwan ROMEE, Boston, MA (US); Cloud PAWELETZ, Boston, MA (US)

(73) Assignee: DANA-FARBER CANCER INSTITUTE, INC., Boston, MA (US)

(21) Appl. No.: 18/863,895

(22) PCT Filed: May 9, 2023
(86) PCT No.: PCT/US2023/066794

§ 371 Date: Nov. 7, 2024

Related U.S. Application Data
(60) Provisional application No. 63/340,217, filed on May 10, 2022.

(51) Int. Cl. A61K 40/42; A61K 40/15; A61K 40/31; A61K 45/06; A61P 35/00; C07K 14/715; C07K 14/725

(52) U.S. Cl. CPC A61K 40/4236; A61K 40/15; A61K 40/31; A61K 45/06; A61P 35/00; C07K 14/7051; C07K 14/7158

(57) ABSTRACT

Disclosed are nucleic acid constructs encoding CXCR3 and a CAR and cells containing same for the treatment of cancers.

### CROSS-REFERENCE TO RELATED APPLICATIONS

This application claims the benefit of priority under 35 U.S.C. § 119 (e) to U.S. Provisional Application No. 63/340,217, filed May 10, 2022, which is incorporated herein by reference in its entirety.

### GOVERNMENT LICENSE RIGHTS

This invention was made with government support under grant number R01CA190394 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Mar. 13, 2023, is named 52095_765001WO_ST.xml and is 78 KB bytes in size.

### BACKGROUND OF THE DISCLOSURE

Activation of the anti-viral stimulator of interferon genes (STING) pathway promotes antitumor immunity. However, STING agonists have yet to achieve clinical success.

Activation of innate antitumor immunity, including the STING pathway, can overcome barriers to therapeutic response such as immune exclusion and exhaustion. STING agonist clinical development has focused primarily on myeloid cell priming of CD8 positive T-cells that reject transplanted mouse syngeneic tumors (Corrales et al., Cell Rep. 11:1018-30 (2015); Sivick et al., Cell Rep. 25:3074-3085 (2018); Amouzegar et al., Cancers (Basel) 13:2695 (2021)). However, STING activation induces stress, cell-cycle arrest, and death in T-cells (Cerboni et al., J. Exp. Med. 214:1769-1785 (2017); Larkin et al., J. Immunol. 199:397-402 (2017); Gulen et al., Nat. Commun. 8:427 (2017)), which has limited its clinical activity. Human tumors also undergo months to years of immune editing, rendering cross-species extrapolation of STING-induced, T-cell killing mechanisms tenuous (O'Donnell et al., Nat. Rev. Clin. Oncol. 16:151-167 (2019)). Despite these limitations, recent mouse studies have explored the complex interplay of STING signaling in the tumor immune microenvironment (TIME), identifying novel effector mechanisms including NK cells and juxtaposing the importance of immune cell versus tumor cell STING activity (Sivick et al., Cell Rep. 25:3074-3085 (2018); Marcus et al., Immunity 49:754-763 (2018); Nicolai et al., Sci Immunol 5:eaaz2738 (2020); Chen et al., Nature 533:493-498 (2016); Sen et al., Cancer Discov. 9:646-661 (2019)).

Chimeric antigen receptor (CAR) expressing cells have demonstrated remarkable efficacy and improved patient outcome, receiving FDA approval for treating liquid tumors. By contrast, the effectiveness of CAR T and CAR NK cell therapies has been less effective in solid tumors due to numerous factors, including the presence of immunosuppressive TME, the vascular barrier, the lack of chemokine gradients and the dysregulation of immune cell trafficking. Therefore, cell therapies effective in treating solid tumors are critically needed.

### SUMMARY OF THE DISCLOSURE

In one aspect, the disclosure provides a nucleic acid construct containing a first nucleic acid containing a promoter operably linked to a nucleic acid encoding a C—X—C Motif Chemokine Receptor 3 (CXCR3), and a second nucleic acid encoding a chimeric antigen receptor (CAR), wherein the CAR contains a ligand binding domain containing a single chain antibody fragment that binds an antigen on a tumor cell, a transmembrane domain, and an intracellular domain containing a signaling domain.

In another aspect, the present disclosure provides a vector containing (e.g., having integrated or cloned therein) the nucleic acid construct.

In another aspect, the disclosure provides a genetically modified immune cell containing the one or more vectors containing the CXCR3-encoding nucleic acid and the CAR-encoding nucleic acid. In some embodiments, the genetically modified immune cell is a NK cell.

In another aspect, the disclosure provides a pharmaceutical composition containing an effective number of genetically modified immune cells expressing the vector and a pharmaceutically acceptable carrier.

In yet another aspect, the disclosure provides a method of treating cancer. The method entails administering to the subject in need thereof an effective amount of the pharmaceutical composition. In some embodiments, the method further entails administering to the subject an effective amount of a STING agonist prior to, substantially contemporaneous with, or subsequent to the administering of the pharmaceutical composition.

Working examples disclosed herein demonstrate that NK cells resist STING-mediated cytotoxicity and that concurrent contact with STING agonists enhance NK cell migration and killing, improving their therapeutic activity. This effect is further enhanced in genetically modified NK cells that overexpress CXCR3 and/or contain an anti-mesothelin CAR. Working examples further show that malignant pleural mesothelioma cells robustly express STING, and that the MPM cells were responsive to STING agonist treatment with adoptive cell therapy ex vivo.

### BRIEF DESCRIPTION OF THE DRAWINGS

FIGS. 1A-D show that STING is highly expressed in immune exhausted MPM. FIG. 1A is a set of dot plots and immunohistochemistry (IHC) microphotographs. FIG. 1B is a bar graph showing immune cell flow cytometry from MPM specimens. FIG. 1C and FIG. 1D are a set of dot plots showing flow cytometry from freshly resected MPM specimens.

FIGS. 2A-2G show that STING agonists promote antitumor immunity in MPM. FIG. 2A is a schematic illustrating the generation of patient derived organotypic spheroids (PDOTS).

FIG. 2B is a set of microphotographs of Hoechst/propidium iodide. FIG. 2C is a set of dot plots showing a summary of percent change in live cell area. FIG. 2D is a Waterfall plot from 35 patient specimens treated with ADU-S100. FIG. 2E is a Waterfall plot showing sample #34 after treatment with ADU-S100. FIG. 2F is a Waterfall plot from 13 patient specimens treated with ADU-S100, TAK-676, or control. FIG. 2G is a dot plot showing percent live/dead for sample #26.

FIGS. 3A-3D show the STING agonists activate tumor cells and fibroblasts. FIG. 3A is a Combined UMAP plot from broad clustering of scRNA sequencing MPM specimen #26. FIG. 3B is a combined UMAP plot for CXCR3 ligands (CXCL9/CXCL10/CXCL11) and mesothelin (MSLN). FIG. 3C is a set of violin plots for select ISG transcripts. FIG. 3D is a set of UMAP plots and a bar plot from combined samples overlayed with contour plots showing the density of cells in each individual sample.

FIGS. 4A-4D show that STING agonists are toxic to T-cells but not NK cells. FIG. 4A is a dot plot showing immune flow cytometry in a MPM. FIG. 4B is a set of bar plots showing cell-titer glow proliferation. FIG. 4C is a set of bar plots showing flow cytometry after 72-hour of treatment. FIG. 4D is a western blot in TILs and NK cells.

FIGS. 5A-5D show that STING agonists enhance NK cell therapies. FIG. 5A is a schematic representation of the NK cell therapy. FIG. 5B is a set of photomicrographs and a dot plot showing representative live/dead IF and quantification from sample #37. FIG. 5C is a set of bar plots showing the quantification of percent change in live cell area. FIG. 5D is a set of representative live/dead IF photomicrographs from sample #32.

FIGS. 6A-6D show that STING agonists enhance adoptive NK cell migration and killing. FIG. 6A is set of photomicrographs and dot plots showing overlayed IF and brightfield images. FIG. 6B is a dot plot showing quantification of triplicate NK cell migration. FIG. 6C is a photomicrograph and dot plot showing representative immunofluorescence modeling NK cell migration. FIG. 6D is a set of flow cytometry plots showing annexin V and live/dead staining.

FIGS. 7A-7E show MPM STING and immune characterization. FIG. 7A is a dot plot of STING IHC. FIG. 7B is a microphotograph of STING IHC in normal pleura. FIG. 7C is a set of microphotographs of Phospho-IRF3 (pIRF3) IHC. FIG. 7D and FIG. 7E are a bar plot and dot plot showing flow cytometry from freshly resected MPM specimens.

FIGS. 8A-8F show STING expression and activation in MPM cell lines. FIG. 8A is a bar plot and western blot showing an CXCL10 ELISA. FIG. 8B is a set of violin plots showing mRNA expression data. FIG. 8C is a dot plot showing a 2′3′ cGAMP ELISA from MPM cell.

FIG. 8D is a set of western blots in MPM cell lines treated with ADU-S100. FIG. 8E is a western blot for STING pathway components. FIG. 8F is a CellTiter-Glo viability assay in MPM cell lines.

FIGS. 9A-9C show ex vivo STING agonist treatment of MPM tumors in PDOTS. FIG. 9A is a set of bar and dot blots showing Hoechst/propidium iodide cell area and percent live/dead.

FIG. 9B is dot and line plots showing CXCL10 ELISA from MPM explants. FIG. 9C is a dot plot showing a summary of MPM PDOTS cell death.

FIGS. 10A-10E show that STING agonists activate tumor cell STING causing CD8-cell killing. FIG. 10A is a set of dot plots showing the correlation between CD8 flow cytometry or CD8 immunofluorescence for MPM PDOTS and subsequent live/dead response. FIG. 10B is a set of dot plots showing Hoechst/propidium iodide cell area and percent live/dead quantification. FIG. 10C is a dot plot showing flow cytometry from a S3 spheroid fragment. FIG. 10D is a set of pie graphs showing MPM immune flow cytometry. FIG. 10E is a set of IRF3 immunofluorescence microphotographs after treatment with ADU-S100.

FIGS. 11A-11D show scRNA seq demonstrates STING activation in tumor cells and fibroblasts. FIG. 11A is a set of heat maps for cluster-defining genes. FIG. 11B is a violin plots for immune cell and fibroblast. FIG. 11C is a volcano plot of differentially expressed genes after 24 hr treatment with ADU-S100. FIG. 11D is a set of UMAP plots for specified transcripts and signatures focused on effector cell cluster 2.

FIGS. 12A-12C show scRNAseq suggests STING agonist toxicity in T-cells. FIG. 12A is a combined UMAP plot from broad clustering of scRNA sequencing of a MPM specimen. FIG. 12B is a violin plots for select NK cell activating/inhibitory transcripts. FIG. 12C is a fraction bar graph showing expression of Treg transcripts.

FIGS. 13A-13G show that STING agonists are toxic to T cells but not NK cells. FIG. 13A is a set of dot and line plots showing CD3/CD56 flow cytometry. FIG. 13B is a set of dot plots showing CellTiter-Glo viability. FIG. 13C is a set of dot plots showing CD4/CD8 flow cytometry. FIG. 13D is a set of dot plots showing time courses of toxicity for CD4+ T-cells. FIG. 13E is a set of dot plots showing mean fluorescence intensity (MFI) from flow cytometry for autophagolysosome vacuoles. FIG. 13F is a western blot from a stage II NSCLC sample. FIG. 13G is a western blot from batch NK cells treated with ADU-S100.

FIGS. 14A-14C show that STING agonists enhance NK cell killing in MPM PDOTs. FIG. 14A is a set of bar plots showing Hoechst/propidium iodide staining. FIG. 14B is a set of IF microphotographs showing Hoechst/propidium iodide live/dead quantification. FIG. 14C is a set of IF microphotographs and bar plots that show live/dead cell area quantification.

FIGS. 15A-15F show that STING agonists enhance NK cell migration and killing. FIG. 15A is a set of four dot plots showing CXCL10 ELISAs. FIG. 15B, is a dot plot showing a Granzyme B ELISA. FIG. 15C is a set of line plots showing flow cytometry with H2591 MPM cells in co-culture with NK cells. FIG. 15D is a set of microphotographs and dot plots showing representative IF images of primary NK cells. FIG. 15E is a set of microphotographs of representative IF images of NK cell migration. FIG. 15F is a set of schematics and IF microphotographs for 3D migration assay.

FIGS. 16A-16C show that STING agonists enhance CAR-NK cell killing in MPM. FIG. 16A is a schematic showing NK cell isolation and transduction with an anti-mesothelin (MSLN) CAR construct. FIG. 16B is a set of flow cytometry plots showing annexin V and live/dead staining of H2591 MPM cells. FIG. 16C is a set of bar plots and histograms showing the quantification of two NK cell donors.

FIGS. 17A-17B show CXCR3 on NK cell surfaces. FIG. 17A is two flow cytometry plots showing CXCR3 staining on primary NK cand JURKAT CXCR3+ cells before and after hCXCL10 treatment. FIG. 17B is a line plot showing the mean fluorescence intensity (MFI) of CXCR3 staining on primary NK and JURKAT CXCR3+ cells.

FIGS. 18A-18B show CXCR3 on NK cell surfaces. FIG. 18A is two flow cytometry plots showing CXCR3 staining on NK92 and JURKAT CXCR3+ cells before and after hCXCL10 treatment. FIG. 18B is a line plot showing the mean fluorescence intensity (MFI) of CXCR3 staining on NK92 and JURKAT CXCR3+ cells.

FIGS. 19A-19D show that CXCR3 is removed from NK cell surfaces. FIG. 19A is a flow cytometry plot showing CXCR3 surface expression of primary NK cells, CAR and CAR-CXCR3, stimulated with recombinant human CXCL10 at 0 and 60 minutes after stimulation. FIG. 19B is a bar plot of the data presented in FIG. 18C, 0 minutes after stimulation represented as 0 and 60 minutes represented as 1. FIG. 19C is a flow cytometry plot showing CXCR3 expression on cNK cells. FIG. 19D is a flow cytometry plot showing CXCR3 staining on CIML NK cells.

FIGS. 20A-20C show cNK cell migration. FIG. 20A is a schematic illustration showing the arrangement of 3D models used to evaluate immune cell interacts and trafficking using a microfluidic device. FIG. 20B is a bar plot showing quantification of experimental triplicates of NK cell migration towards H226 MPM cancer cell spheroids. FIG. 20C is two representative immunofluorescence (IF) images of primary CAR-expressing NK cells with and without CXCR3 overexpression labeled with red cell tracer migrating towards H226 MPM cancer cell spheroids after 3 days of culture (scale bar=150 μm).

FIGS. 21A-21B show cNK cell migration. FIG. 21A is a bar plot showing quantification of experimental triplicates of NK cell migration towards H2591 cancer cell spheroids. FIG. 21B is two representative IF images of primary CAR-expressing NK cells with and without CXCR3 overexpression labeled with red cell tracer migrating towards H2591 cancer cell spheroids after 3 days of culture (scale bar=150 μm).

FIGS. 22A-22B show cNK cell migration. FIG. 22A is a bar plot showing quantification of experimental triplicates of NK cell with and without CXCR3 overexpression migration towards H226 cancer cell spheroids treated with and without ADU-S100. FIG. 22B is four microphotographs of the data quantified in FIG. 22A.

FIGS. 23A-23D show cNK cell migration. FIG. 23A is a bar plot showing quantification of experimental triplicates of NK cell with and without CXCR3 overexpression migration towards H2591 cancer cell spheroids treated with and without ADU-S100. FIG. 23B is four microphotographs of the data quantified in FIG. 23A. FIG. 23C is a bar plot showing quantification of experimental triplicates of NK cell migration towards H2591 cancer cell spheroids after 3-day treatment with control (dH2O) or 50 mM ADU-S100. FIG. 23D is representative IF images of primary CAR-expressing NK cells with and without CXCR3 overexpression (top images) with and without ADU-S100 treatment (bottom images) labeled with red cell tracer migrating towards H2591 cancer cell spheroids (scale bar=150 μm).

FIGS. 24A-24D show CAR-NK cell migration and killing. FIG. 24A is a bar plot showing CAR-NK cell migration towards H226 MPM cells with and without CXCR3 overexpression. FIG. 24B is two microphotographs of the data quantified in FIG. 24A. FIG. 24C is a bar plot showing CAR-NK cell killing H226 MPM cells with and without CXCR3 overexpression. FIG. 24D is two microphotographs of the data quantified in FIG. 24B.

FIGS. 25A-25B show CAR-NK cell migration. FIG. 25A is a bar plot showing CAR-NK cell migration with and without ADU-S100 treatment. FIG. 25B is a set of microphotographs of the data quantified in FIG. 25A.

FIGS. 26A-26B show CAR staining on NK cells from donor #27.

FIGS. 27A-27B show CAR staining on NK cells from donor #28.

FIGS. 28A-28B show CXCR3 staining on NK cells from donor #27.

FIGS. 29A-29B show CXCR3 staining on NK cells from donor #28.

FIGS. 30A-30B show two constructs used to produce genetically modified immune cells. FIG. 30A is an illustration that shows a vector containing an anti-Mesothelin CAR nucleic acid. FIG. 30B is an illustration that shows a vector containing an anti-Mesothelin CAR nucleic acid and a CXCR3 nucleic acid.

### DETAILED DESCRIPTION OF THE DISCLOSURE

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated to facilitate the understanding of the present disclosure.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, reference to “a construct” includes a use case with more than one construct, and the like.

Unless stated otherwise, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

The term “approximately” as used herein refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed disclosure.

The terms “overexpression”, “overexpressing”, and “overexpressed” are used interchangeably herein to be 30% or more increase of protein or messenger RNA as compared with an appropriate control when referring to CXCR3 expression.

### Nucleic Acid Constructs

In one aspect, the disclosure provides a nucleic acid construct containing a first nucleic acid containing a first promoter operably linked to a nucleic acid encoding a C—X—C Motif Chemokine Receptor 3 (CXCR3) and a second nucleic acid encoding a chimeric antigen receptor (CAR), wherein the CAR contains a ligand binding domain containing a single chain antibody fragment that binds an antigen on a tumor cell, a transmembrane domain, and an intracellular domain containing a signaling domain. In some embodiments, the second nucleic acid is operably linked to a second promoter, which may be the same or different from the first promoter. In some embodiments, a third nucleic acid encoding a self-cleaving peptide is disposed between the first and second nucleic acids, and the first promoter drives expression of the CXCR3 nucleic acid, the self-cleaving peptide, and the CAR nucleic acid.

The term “nucleic acid” as used herein refers to a polymer of nucleotides, each of which are organic molecules consisting of a nucleoside (a nucleobase and a five-carbon sugar) and a phosphate. The term nucleotide, unless specifically sated or obvious from context, includes nucleosides that have a ribose sugar (i.e., a ribonucleotide that forms ribonucleic acid, RNA) or a 2′-deoxyribose sugar (i.e., a deoxyribonucleotide that forms deoxyribonucleic acid, DNA). Nucleotides serve as the monomeric units of nucleic acid polymers or polynucleotides. The four nucleobases in DNA are guanine (G), adenine (A), cytosine (C) and thymine (T). The four nucleobases in RNA are guanine (G), adenine (A), cytosine (C) and uracil (U). Nucleic acids are linear chains of nucleotides (e.g., at least 3 nucleotides) chemically bonded by a series of ester linkages between the phosphoryl group of one nucleotide and the hydroxyl group of the sugar (i.e., ribose or 2′-deoxyribose) in the adjacent nucleotide.

The term “promoter” as used herein refers to a nucleic acid that regulates, directly or indirectly, the transcription of a corresponding nucleic acid coding sequence to which it is operably linked. A promoter may function alone to regulate transcription, or it may act in concert with one or more other regulatory sequences (e.g., enhancers or silencers, or regulatory elements that may be present in the vector). Promoters are located near the transcription start sites of open reading frames, on the same strand and upstream on the DNA (towards the 5′ region of the sense strand). Promoters typically range from about 100-1000 base pairs in length.

The term “operatively linked” as used herein is to be understood that a nucleic acid is spatially situated or disposed in the nucleic acid construct relative to a promoter to drive the expression of the protein encoded by the nucleic acid (e.g., CXCR3).

### CXCR3

CXCR3 is a chemokine receptor that induces cellular responses that are involved in immune cell trafficking. As demonstrated in the working examples below, genetically modified immune cells that contains a nucleic acid encoding CXCR3 have increased migration into TME.

CXCR3 is a G protein-coupled receptor that binds three chemokines, known as monokine induced by interferon-g (Mig/CXCL9), interferon-γ-inducible 10 kDa protein (IP10/CXCL10) and interferon-inducible T cell a-chemoattractant (I-TAC/CXCL11). Binding of chemokines to CXCR3 induces cellular responses including integrin activation, cytoskeletal changes, and chemotactic migration.

The amino acid sequence of a representative CXCR3 is provided at NCBI Accession No. NP_001495, version NP_001495.1, incorporated herein by reference, and set forth in the sequence listing as SEQ ID NO: 1 The nucleic acid sequence encoding the CXCR3 protein (SEQ ID NO: 1) is provided at NCBI Accession No. NC_000023, version NC_000023.11, incorporated herein by reference, and set forth in the sequence listing as SEQ ID NO: 2. The nucleic acid sequence encoding another representative CXCR3 is set forth in the sequence listing as SEQ ID NO: 3.

### CAR

The CAR binds an antigen on the surface of a cancer cell. The CAR contains a ligand binding domain containing a single chain antibody fragment that binds an antigen on the surface of a cancer (e.g., tumor cell), a transmembrane domain, and an intracellular domain containing a signaling domain. In some embodiments, the ligand binding domain is an antibody fragment (e.g., a scFv).

In some embodiments, the CAR is specific for, and binds a malignant pleural mesothelioma (MPM) antigen. In some of these embodiments, the MPM antigen is mesothelin. In some embodiments, the CAR ligand binding domain is derived from an anti-mesothelin antibody, antibody fragment, or derivative thereof. In some embodiments, the CAR ligand binding domain is derived from YP218, amatuximab, RC88, 19C3, 3C10, or 7B1. The nucleic acid sequences of YP218 VH (SEQ ID NO: 4) and VL (SEQ ID NO: 5), amatuximab VH (SEQ ID NO: 6) and VL (SEQ ID NO: 7), RC88 VH (SEQ ID NO: 8) and VL (SEQ ID NO: 9), 19C3 VH (SEQ ID NO: 10) and VL (SEQ ID NO: 11), 3C10 VH (SEQ ID NO: 12) and VL (SEQ ID NO: 13), and 7B1 VH (SEQ ID NO: 14) and VL (SEQ ID NO: 15) are set forth in the sequence listing. The amino acid sequences of YP218 VH (SEQ ID NO: 16) and VL (SEQ ID NO: 17), amatuximab VH (SEQ ID NO: 18) and VL (SEQ ID NO: 19), RC88 VH (SEQ ID NO: 20) and VL (SEQ ID NO: 21), 19C3 VH (SEQ ID NO: 22) and VL (SEQ ID NO: 23), 3C10 VH (SEQ ID NO: 24) and VL (SEQ ID NO: 25), and 7B1 VH (SEQ ID NO: 26) and VL (SEQ ID NO: 27) are set forth in the sequence listing.

In some embodiments, the CAR ligand binding domain contains the VH having the amino acid sequence of SEQ ID NO: 4. In some embodiments, the CAR ligand binding domain contains the VL having the amino acid sequence of SEQ ID NO: 5.

Additional anti-mesothelin antibodies and mesothelin-binding fragments thereof are known in the art. See, e.g., U.S. Pat. Nos. 7,081,518, 7,943,133, 8,460,660, 8,911,732, 9,272,002, 9,719,996, 10,022,452, 10,183,993, 10,793,641, and 10,919,975, and U.S. Patent Application Publications 2009/0047211, 2015/0252118, and 2022/0056147.

The transmembrane domain of the CAR connects the CAR ligand binding domain to the intracellular domain. In some embodiments, the transmembrane domain is directly connected to the CAR ligand binding domain. In some embodiments, the transmembrane domain is derived from CD3α, CD3β, CD3γ, CD3ζ, CD3ε, CD4, CD5, CD8α, CD9, CD16, CD22, CD28, CD33, CD37, CD45, CD64, CD80, CD86, CD134, CD137 (4-1BB or TNF Receptor Superfamily Member 9 (TNFRSF9)), CD154, FcεRIα, FcεRIβ, FcεRIγ, ICOS, KIR2DS2, MHC class I, MHC class II, or NKG2D. Amino acid sequences of representative transmembrane domains are set forth in the sequence listing as SEQ ID Nos: 14-18.

The amino acid sequence of a naturally occurring transmembrane domain may be modified by an amino acid substitution to avoid binding of such regions to the transmembrane domain of the same or different surface membrane proteins to minimize interactions with other members of a receptor complex. See, e.g., U.S. Patent Application Publication 2021/0101954; Soudais et al., Nat. Genet. 3:77-81 (1993); Muller et al., Front. Immunol. 12:639818-13 (2021); and Elazar et al., elife 11:e75660-29 (2022).

In some embodiments, the CAR further includes a hinge domain disposed between the ligand binding domain and the transmembrane domain. A hinge domain may provide flexibility in terms of allowing the ligand binding domain to obtain an optimal orientation for antigen-binding, thereby enhancing antitumor activities of the genetically modified immune cell expressing the CAR. In some embodiments, the hinge domain is derived from IgA, IgD, IgE, IgG, or IgM. In some embodiments, the hinge domain is derived from CD3ζ, CD4, CD8α, CD28, IgG1, IgG2, or IgG4, representative amino acid sequences of which are set forth in the sequence listing as SEQ ID Nos: 19-25, respectively.

The intracellular domain of the CAR contains a signaling domain that enables intracellular signaling and immune cell function. The signaling domain may include a primary signaling domain and/or a co-stimulatory signaling domain. In some embodiments, the intracellular domain is capable of delivering a signal approximating that of natural ligation of an ITAM-containing molecule or receptor complex such as a TCR receptor complex.

The intracellular domain contains a signaling domain that enables intracellular signaling and immune cell function. The signaling domain may include a primary signaling domain and/or a co-stimulatory signaling domain. In some embodiments, the intracellular domain includes one or more phosphorylatable intracellular motifs (ITAMs) capable of delivering an immune activating signal. In some embodiments, the intracellular domain is capable of delivering a signal approximating that of natural ligation of an ITAM-containing molecule or receptor complex such as a TCR receptor complex.

In some embodiments, the signaling domain includes a plurality, e.g., 2 or 3, costimulatory signaling domains, e.g., selected from 4-1BB, CD3ζ, CD28, CD27, ICOS, and OX40. In some embodiments, the signaling domain may include a CD3ζ domain as a primary signaling domain, and any of the following pairs of co-stimulatory signaling domains from the extracellular to the intracellular direction: 4-1BB-CD27; CD27-4-1BB; 4-1BB-CD28; CD28-4-1BB; OX40-CD28; CD28-OX40; 4-1BB-CD3ζ; CD3ζ-4-1BB; CD28-CD3ζ; and CD3ζ-CD28. In some embodiments the primary signaling domain is derived from CD3, CD27, CD28, CD40, KIR2DS2, MyD88, or OX40. In some embodiments, the co-stimulatory signaling domain is derived from one or more of 4-1BB (CD137; TNFRSF9), CD3γ, CD3ζ, CD3ε, CD3ζ, CD4, CD5, CD8α, CD9, CD16, CD22, CD27, CD28, CD33, CD37, CD40, CD45, CD68, CD72, CD80, CD86, CD154, CLEC-1, DAP10 (hematopoietic cell signal transducer ((HCST)), DAP12 (TYROBP), Dectin-1, FcεRI, FcγRI, FcγRII, FcγRIII, IL-2RB, ICOS, KIR2DS2, MyD88, OX40, and ZAP70. Amino acid sequences of representative signaling domains are set forth in the sequence listing as SEQ ID Nos: 26-43, respectively.

In some embodiments, the signaling domain is derived from CD3ζ and the co-stimulatory domain is derived from 4-1BB. In some embodiments, the signaling domain is derived from CD3ζ and the co-stimulatory domain is derived from CD28. In some embodiments, the signaling domain is derived from CD3, and the co-stimulatory domain is derived from 4-1BB and CD28. Amino acid sequences of representative 4-1BB and CD28 are set forth in SEQ ID NO: 26 and SEQ ID NO: 32, respectively, and additional isoforms of CD28 are provided in the sequence listing as SEQ ID Nos: 44-46.

The expression of the first nucleic acid encoding a CXCR3 and expression of the second nucleic acid encoding a CAR are controlled by one or more promoters, which may be a natural or synthetic. In some embodiments, a third nucleic acid encoding a self-cleaving peptide or an internal ribosome entry site (IRES) is disposed between the first and the second nucleic acids. In these embodiments, the first nucleic acid and the second nucleic acid are controlled by the same promoter. In some embodiments, the second nucleic acid is controlled by a second promoter different from the first promoter.

In some embodiments, the first promoter is a strong promoter that overexpresses the nucleic acid to which it is operatively linked. Overexpression can be achieved by providing a vector encoding the protein controlled by a constitutive promoter, or by removing repressors, adding multiple copies of the gene to the cell, or up regulating the endogenous gene, and the like. In some embodiments, one or both of the promoters are derived from the elongation factor 1 Alpha (EF-1α), cytomegalovirus (CMV), β-actin, a simian virus 40 (SV40) early promoter, human phosphoglycerate kinase (PGK), RPBSA (synthetic, from Sleeping Beauty), or CAG (synthetic, CMV early enhancer element, chicken β-Actin, and splice acceptor of rabbit β-Globin) promoter. The term “derived from” as used herein when referring to proteins or nucleic acids refers to a protein or nucleic acid that originates from another, parental protein or nucleic acid. The derived protein or nucleic acid has a sequence that may be identical to the parental sequence, may be a portion of the parent sequence, or may have at least one variant from the parent sequence. Variants may include amino acid and nucleotide substitutions, insertions, or deletions. Thus, for example, an amino acid sequence derived from a parent sequence may be identical for a specific range of amino acids of the parent but does not include amino acids outside that specific region.

In some embodiments, a promoter may have a core region located close to the beginning of the nucleic acid coding sequence. In some embodiments, the promoter is modified relative to a native promoter. One modification entails the removal of methylation sensitive sites (e.g., a cytosine nucleotide is followed by a guanine nucleotide, or “CpG”). Another modification entails the addition of a regulatory sequence that binds DNA methylation repressive transcriptional factors. In some embodiments, the expression vector includes A/T-rich, nuclear matrix interacting sequences, known as scaffold matrix attachment regions (S/MAR), which may enhance transformation efficiency and improve the stability of transgene expression.

In some embodiments, the first and the second promoters are derived from EF-1a. In some embodiments, the first promoter is derived from CMV, and the second promoter is derived from EF-1α. The sequence of the EF-1α promoter is provided at NCBI Accession No. J04617.1. Sequences of the CMV promoter from different CMV isolates are provided at NCBI Accession Nos. AY218848, AF477200, M64754, and AF286076. The sequence of the PGK promoter is provided at NCBI Accession No. NC_000023.11, range 78104248 to 78129295. The sequence of the RPBSA promoter is provided in NCBI Accession No. MN811119.1. The sequence of the CAG promoter is provided in NCBI Accession No. MG763233.1.

In some embodiment, the nucleic acid construct contains self-cleaving polypeptide-encoding nucleic acid disposed between the CXCR3-encoding nucleic acid and the CAR encoding nucleic acid. Nucleic acid sequences of representative self-cleaving polypeptides are set forth in the sequence listing as SEQ ID NOs 476-49.

In some embodiments, the nucleic acid construct contains a selection marker to aid in isolation, capture or detection. A selection marker typically entails addition of an in-frame nucleic acid that will be translated into amino acids along with the protein to which the it is attached. Representative examples of selection markers include enhanced green fluorescent protein (EGFP) (SEQ ID NO: 50), AU1 epitope (SEQ ID NO: 51), AU5 epitope (SEQ ID NO: 52), polyhistidine (SEQ ID NO: 53), FLAG epitope (SEQ ID NO: 54), FLAG His tag (SEQ ID NO: 55), histidine affinity tag (HAT) (SEQ ID NO: 56), herpes simplex virus (HSV) epitope (SEQ ID NO: 57), human influenza hemagglutinin (HA), glutathione S-transferase (GST), KT3 epitope, maltose binding protein (MBP), Bacteriophage T7 epitope, myc tags. Amino acid sequences of representative selection markers are listed in the sequence listing as SEQ ID NOs: 50-57.

### Vectors

The nucleic acids encoding the CXCR3, and CAR may be introduced into an immune cell by the same or separate vectors. The nucleic acid constructs are introduced into an immune cell by a suitable vector. A vector is configured so as to contain additional regulatory elements necessary to effect transport into the immune cell and effect expression of the nucleic acid(s) after transformation. Such elements include an origin of replication or promoter, a poly-A tail sequence a selectable marker, one or more suitable sites for the insertion of nucleic acid sequences, such as a multiple cloning site (MCS), and the selectable marker, and additional optional regulatory elements.

In some embodiments, the vector is a viral vector, for example, a retroviral vector, a lentiviral vector, an adenoviral vector, a herpesvirus vector, an adenovirus, or an adeno-associated virus (AAV) vector. As used herein, the term “lentiviral vector” is intended to mean an infectious lentiviral particle. Lentivirinae or lentivirus, is a subfamily of enveloped retrovirinae or retroviruses, that are distinguishable from other viruses by virion structure, host range, and pathological effects. An infectious lentiviral particle will be capable of invading a target host cell, including infecting, and transducing non-dividing cells and immune cells. Lentiviral characteristics include, for example, infecting or transducing non-dividing host cells, including immune cells.

In some embodiments, the vector is a recombinant lentivirus comprising a recombinant genome comprising, between the LTR 5′ and 3′ lentiviral sequences, a lentiviral encapsulation psi sequence, an RNA nuclear export element, a transgene, a promoter and/or a sequence favouring the nuclear import of RNA, as well as a mutated integrase preventing the integration of its genome into the genome of a host cell. The construction of lentiviral vectors has been described, for example, in U.S. Pat. Nos. 5,665,577, 5,981,276, 6,013,516, 7,090,837, 8,119,119 and 10,954,530.

In some embodiments, the vector is a non-integrative and non-replicative recombinant lentivirus vector. The construction of lentiviral vectors has been described, for example, in U.S. Pat. Nos. 5,665,577, 5,981,276, 6,013,516, 7,090,837, 8,119,119 and 10,954,530. Lentivirus vectors include a defective lentiviral genome, i.e., in which at least one of the lentivirus genes gag, pol, and env, has been inactivated or deleted.

A lentiviral vector also can exhibit functions additional to, or different from, a naturally occurring lentivirus. For example, a lentiviral vector can be modified to change or reduce a lentivirus characteristic. A lentiviral vector also can be modified to exhibit characteristics of one or more other retroviruses, retroviral vectors, host cells, or heterologous cells. Modifications can include, for example, pseudotyping, modifying binding and/or fusion functions of the envelope polypeptide, incorporating heterologous, chimeric, or multifunctional polypeptides into the vector, incorporating non-lentivirus genomes, or incorporating heterologous genes into the lentiviral vector genome.

The terms “pseudotyping”, “pseudotyped”, “pseudotyped vector”, and “pseudotyped vector particle” are used herein to refer to a vector bearing components (e.g., envelop or capsid) from more than one source. The sources may be from a heterologous virus or non-viral proteins. Non-viral proteins may include antibodies and antigen-binding fragments thereof. A representative pseudotyped vector is a vector bearing non-glycoprotein components derived from a first virus and envelope glycoproteins derived from a second virus. The host range of a pseudotyped vector may thusly be expanded or altered depending on the type of cell surface receptor bound by the glycoprotein derived from the second virus.

In some embodiments, the lentiviral vector is pseudotyped with a baboon envelop (BaEV) glycoprotein (BaEV-gp). The amino acid sequence of a representative BaEV-gp is set forth in the sequence listing as SEQ ID NO: 58. The nucleic acid sequence encoding the BaEV-gp (SEQ ID NO: 58) is set forth as SEQ ID NO: 59. Additional BaEv pseudotyped lentivirus vectors are known in the art. See, e.g. Levy et al., J. Thromb. Haemost. 14:2478-2492 (2016), Costa et al., Leukemia 31:977-980 (2017), and Bari et al., Front. Immunol. 10:2001 (2019). The nucleic acid sequence of a representative a BaEV vector is set forth in the sequence listing as SEQ ID NO: 60.

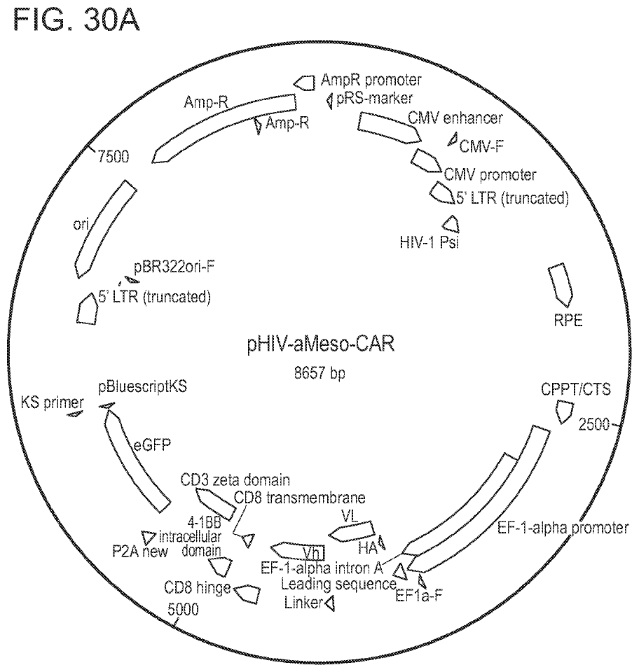

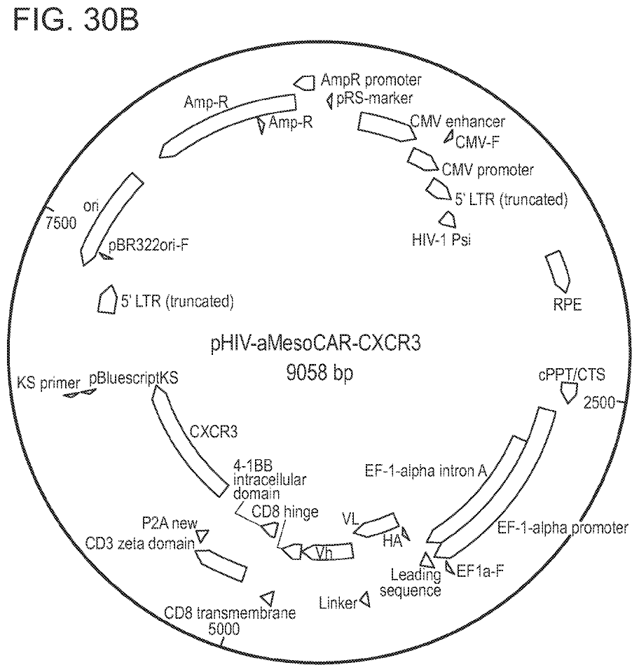

In some embodiments, the vector contains a plx307-based nucleic acid construct. In some embodiments, the vector contains a pHIV-based nucleic acid construct. The nucleic acid sequence of a representative vector containing a pHIV-based nucleic acid construct containing a CAR-encoding nucleic acid that binds mesothelin (pHIV-aMesoCAR-GFP) is set forth in the sequence listing as SEQ ID NO: 61. The nucleic acid sequence of a representative vector containing a pHIV-based nucleic acid construct encoding a CXCR3 and a CAR that binds mesothelin (pHIV-aMesoCAR-CXCR3; illustrated in FIG. 30B) is set forth in the sequence listing as SEQ ID NO: 62.

In some embodiments, the vector contains a pCMV-based nucleic acid construct. The nucleic acid sequence of a representative vector containing a pCMV-based nucleic acid construct (pCMV-dR8.91) is set forth in the sequence listing as SEQ ID NO: 63. In some embodiments, the vector contains a pAdv-based nucleic acid construct. The nucleic acid sequence of a representative vector containing a pAdv-based nucleic acid construct (pAdvAntage) is set forth in the sequence listing as SEQ ID NO: 64.

### Cells

One aspect of the present disclosure is a genetically modified (or transformed) immune cell containing a vector that contains a nucleic acid construct encoding the CXCR3 and CAR. As used herein, “immune cell” refers to a cell of hematopoietic origin functionally involved in the initiation and/or execution of innate and/or adaptative immune response. Representative examples of immune cells include natural killer (NK) cells, T cells, macrophages, and dendritic cells. Combinations of different genetically modified immune cells may be used. In some embodiments, the genetically modified immune cells are NK cells. In some embodiments, the genetically modified immune cells are from a NK cell line, primary NK cells, stem cell-derived NK cells, cord blood-derived NK cells, peripheral blood mononuclear cells (PBMC)-derived NK cells, memory-like NK cells, or induced memory like NK cells. Suitable NK cell lines suitable for the present methods include NK-92, NKG, NKL, KHYG-1, YT, NK-YS, SNK-6, IMC-1, YTS, NKL cells, and high affinity NK (haNK, an NK/T cell lymphoma cell line).

In some embodiments, the genetically modified immune cells are memory-like NK cells. Memory-like NK cells may be generated by harvesting NK cells from a subject, for example purified from a peripheral blood sample, stimulated with cytokines (e.g., IL-12, IL-15, and IL-18) for a suitable period of time (e.g., between about 12 hours to less than 7 days), cytokines removed, and transduced to express a CXCR3 and a CAR. In some embodiments, the genetically modified immune cells are cytokine-induced memory-like (CIML) NK cells. CIML NK cells may be produced by stimulating NK cells with a one or more, but typically in combination, of IL-12, IL-15, and IL-18. See, e.g., Cooper et al., Proc. Natl Acad. Sci. USA 106:1915-9 (2009); Ni et al., J. Exp. Med. 209:2351-65 (2012); Keppel et al., J. Immunol. 190:4754-62 (2013).

In some embodiments, the cells are T cells. In some embodiments, the T cells are naive T cells, memory stem cell T cells, central memory T cells, effector memory T cells, helper T cells, CD4+ T cells, CD8+ T cells, CD8/CD4+ T cells, αβ T cells, γδ T cells, and natural killer T (NKT) cells, and Th17 T cells. T cell isolation and fractionation into T cell subsets are known in the art. See, for example, U.S. Pat. Nos. 10,507,219, 11,135,245, and 11,242,376, and U.S. Patent Application Publications 2013/0060011, 2019/0276540, 2020/0347350, and 2021/0106622.

Methods of introducing the vectors containing a nucleic acid construct into immune cells are known in the art. See, e.g., U.S. Pat. Nos. 7,399,633, 7,575,925, 10,072,062, 10,370,452, and 10,829,735, and U.S. Patent Application Publications 2019/0000880 and 2021/0407639.

In some embodiments, a lentiviral vector is transduced into immune cells. In other embodiments, the method entails the use of gamma retroviral vectors. See, e.g., U.S. Pat. Nos. 9,669,049, 11,065,311, and 11,230,719. In some embodiments, the method entails the use of Adenovirus, Adeno-associated virus (AAV), dsRNA, ssDNA, or dsRNA to deliver the nucleic acid construct. See, e.g., U.S. Pat. No. 10,563,226, and U.S. Patent Application Publications 2019/0225991, 2020/0080108, and 2022/0186263.

### Pharmaceutical Compositions

Pharmaceutical compositions of the disclosure include effective numbers of genetically modified immune cells and a pharmaceutically acceptable carrier. The term “effective number of genetically modified immune cells” (which indirectly includes a corresponding amount of the CXCR3 and CAR) as used herein refers to a sufficient number of the genetically modified immune cells that contain nucleic acids encoding CXCR3 and a CAR to provide the desired effect.

Compositions may be provided as sterile liquid preparations, e.g., isotonic aqueous solutions, suspensions, emulsions, dispersions, or viscous compositions, which may be buffered to a selected pH. Liquid carriers include aqueous or non-aqueous carriers alike. Representative examples of liquid carriers include saline, phosphate buffered saline, a soluble protein, dimethyl sulfoxide (DMSO), polyol (e.g., glycerol, propylene glycol, liquid polyethylene glycol, and the like) and suitable mixtures thereof. In some embodiments, the liquid carrier includes a protein dissolved or dispersed therein, representative examples include serum albumin (e.g., human serum albumin, recombinant human albumin), gelatin, and casein. The compositions are typically isotonic, i.e., they have the same osmotic pressure as blood. Sodium chloride and isotonic electrolyte solutions (e.g., Plasma-Lyte®) may be used to achieve the desired isotonicity. Depending on the carrier and the genetically modified immune cells, other excipients may be added, e.g., wetting, dispersing, or emulsifying agents, gelling and viscosity enhancing agents, preservatives and the like as known in the art.

### Cancer

In some aspects, the present disclosure is directed to treating cancer in a subject. The method entails administering to the subject in need thereof an effective number of genetically modified immune cells containing a nucleic acid construct that contains a first nucleic acid encoding CXCR3 and a second nucleic acid encoding a CAR (also referred to herein as “genetically modified immune cells”). The term “cancer” as used herein refers to a disease characterized by uncontrolled cellular proliferation, reduced cellular apoptosis, and spread of abnormal cells that invade and destroy non-cancerous tissues. Cancer cells may be in the form of a tumor (i.e., a solid tumor), or may exist alone within a subject also referred to as liquid tumors. The term cancer includes pre-malignant as well as malignant cancers.

In some embodiments the cancer is a solid tumor. Solid tumors are highly heterogenic due to the different types of tissue a solid tumor develops in the characteristics of tumor growth. In some embodiments, the solid tumor is a sarcoma or a carcinoma. In some embodiments, the cancer is MPM. Some embodiments are directed to a method of treating MPM by administering to a subject in need thereof an effective amount of NK cells containing a nucleic acid construct with a CXCR3 and a CAR or a pharmaceutical composition thereof. In some embodiments, the method further entails administering to the subject an effective amount of a STING agonist prior to, substantially contemporaneous with, or subsequent to the administering of the NK cells or the pharmaceutical composition thereof.

In some embodiments, the cancer comprises hypermethylation of the Cyclic GMP-AMP Synthase (cGAS) or STING gene promoters. In some of these embodiments, the cancer is bladder urothelial carcinoma (BLCA), breast invasive carcinoma (BRCA), cervical squamous cell carcinoma and endocervical adenocarcinoma (CESC), cervical precancerous lesions (CPL), colon adenocarcinoma (COAD), gliomas (e.g., glioblastoma), head and neck squamous cell carcinoma (HNSC), kidney renal clear cell carcinoma (KIRC), kidney renal papillary cell carcinoma (KIRP), liver hepatocellular carcinoma (LIHC), lung adenocarcinoma (LUAD) lung squamous cell carcinoma (LUSC), melanomas, ovarian cancers, pancreatic adenocarcinoma (PAAD), prostate adenocarcinoma (PRAD), rectum adenocarcinoma (READ), sarcoma (SARC), skin cutaneous melanoma (SKCM), stomach adenocarcinoma (STAD), thyroid carcinoma (THCA), and uterine corpus endometrial carcinoma (UCEC). See, Konno et al., Oncogene 37:2037-2051 (2018), de Queiroz et al., Mol. Cancer Res. 17:974-986 (2019), Huang et al., Front. Genet. 10:1-11 (2019), Falahat et al., Proc. Natl. Acad. Sci. U.S.A. 118:1-9 (2021), Low et al., Cancer Cell 40:439-440 (2022).

In some embodiments, the cancer has high basal STING expression, also as referred herein as STING⁺. As used herein, term “high basal” expression of a gene refers to elevated expression of a gene in a disease state as compared to a reference, non-diseased state. In some embodiments, the STING⁺ cancer is melanoma (e.g., malignant melanoma), gastric cancer, liver cancer (e.g., hepatocellular carcinoma (HCC)), lung cancer (e.g., non-small cell lung cancer (NSCLC)), bladder cancer, colorectal cancer, or breast cancer. Additional cancers in which STING has been shown to play a role are known in the art and include leukemia (e.g., acute myeloid leukemia), lymphoma (e.g., malignant lymphoma), breast cancer, colorectal cancer, glioma, head and neck squamous cell carcinoma, lung cancer, melanoma, nasopharyngeal carcinoma, ovarian cancer, pancreatic cancer, prostate cancer, and tongue squamous cell carcinoma. See, Zhu et al., Mol. Cancer 18 (1): 152 (2019).

The terms “treat”, “treating”, and “treatment” as used herein refer to any type of intervention, process performed on, or the administration of an active agent to the subject in need thereof with the therapeutic objective (“therapeutic effect”) of reversing, alleviating, ameliorating, inhibiting, diminishing, slowing down, arresting, stabilizing, or preventing the onset, progression, development, severity or recurrence of a symptom, complication or condition, or biochemical indicia associated with a cancer.

The term “subject” (or “patient”) as used herein includes all members of the animal kingdom prone to or suffering from the indicated cancer. Therefore, a subject “having a cancer” or “in need of” treatment according to the present disclosure broadly embraces subjects who have been positively diagnosed, including subjects having active disease who may have been previously treated with one or more rounds of therapy, and subjects who are not currently being treated (e.g., in remission) but who might still be at risk of relapse, and subjects who have not been positively diagnosed but who are predisposed to a cancer (e.g., on account of the basis of prior medical history and/or family medical history, or who otherwise present with a one or more risk factors such that a medical professional might reasonably suspect that the subject was predisposed to cancer).

### Administration

The number of genetically modified immune cells administered to a subject will vary between wide limits, depending upon the location, type, and severity of the cancer, the age, body weight, and condition of the individual to be treated, etc. A physician will ultimately determine appropriate number of cells and doses to be used. Typically, the genetically modified immune cells will be given in a single dose. In some embodiments, the effective number of the genetically modified immune cells is between approximately 1×10⁵ to approximately 1×10¹⁰ cells per subject. In some embodiments, the effective number of the genetically modified immune cells is between approximately 1×10⁵ to approximately 6×10⁸ cells per kg of subject body weight.

Compositions containing a therapeutically effective number of the genetically modified immune cells may be administered to a subject for the treatment of a cancer by any medically acceptable route. The genetically modified immune cells are typically delivered intravenously, although they may also be introduced into other convenient sites (e.g., intratumorally to an affected organ or tissue) or modes, as determined by an attending physician. Expansion and differentiation agents can be provided prior to, during or after administration of the cells to increase differentiation, expansion, or persistence of the genetically modified immune cells (e.g., NK cells).

In some embodiments, the genetically modified immune cells are administered as a single intravenous infusion over a period of time. Representative infusion times are 30 minutes, 60 minutes, and 90 minutes. In some embodiments, the infusion time is between 30 and 60 minutes. In some embodiments, the first administration is infused into a patient for 90 minutes and subsequent administrations are infused into a patient for 30 minutes.

### Combination Therapy

In some embodiments, the present methods include co-administration of a STING agonist. The term “co-administered” includes substantially contemporaneous administration, by the same or separate dosage forms, or sequentially, e.g., as part of the same treatment regimen or by way of successive treatment regimens. The sequence and time interval may be determined such that the co-administered therapies can act together (e.g., synergistically to provide an increased benefit than if they were administered otherwise). For example, the therapeutics may be administered at the same time or sequentially in any order at different points in time; however, if not administered at the same time, they may be administered sufficiently close in time so as to provide the desired therapeutic effect, which may be in a synergistic fashion.

In some embodiments, the genetically modified immune cells of the present disclosure are used in conjunction with a STING agonist. In some embodiments, the STING agonist is ADU-S100, TAK-676, BI-STING, BMS-986301, GSK532, DMXAA (ASA-404), GSK3745417, JNJ-4412, MK-1454, SB11285, 3′3′-scylic AIMP, ALG-031048, E7766, JNJ-′6196, MK-2118, MSA-1, MSA-2, SNX281m SR-717, KAT676, TTI-10001, XMT-2056, CRD-5500, c-di-AMP, synthetic cyclic dinucleotide (DCN) molecules, analogs thereof, or a combination thereof. In some embodiments, the STING agonist is ADU-S100 or TAK-676. In some embodiments, the STING agonist is delivered by intratumoral injection or systemically (i.e., intravenously). See, Woodward et al., Science 328:1703-5 (2010), Motedayen Aval et al., J. Clin. Med. 9:3323 (2020) and U.S. Pat. Nos. 11,285,131 and 11,312,772, and U.S. Patent Application Publications 2018/0028553, 2019/0328762, 2020/0330556, and 2021/0170043.

In some embodiments, the present methods include co-administration of the genetically modified immune cells, and another anti-cancer agent, with or without the STING agonist. Representative examples of additional anti-cancer agents are set forth below.

Anti-cancer agents that may be used in combination with the inventive cells are known in the art. See, e.g., U.S. Pat. No. 9,101,622 (Section 5.2 thereof). An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

In some embodiments, the genetically modified immune cells of the present disclosure, or the genetically modified immune cells in combination with the STING agonist, are used in combination with a type I IFN agonist. In some embodiments, the type I INF agonist is a recombinant synthetic type I INF protein, for example Interferon alfacon-1 (Infergen®), recombinant Interferon Alfa-2b (Intron AR, Roferon®-A), Interferon beta-1b (Betaseron®, Extavia®, Rebif®, Avonex®), interferon alpha-2c (Berofor Alpha®), interferon alfa-n4 (Alferon N®), or pegylated IFN, e.g., peginterferon beta-1a (Plegridgy®).

In some embodiments, the genetically modified immune cells of the present disclosure are used in conjunction with a DNA methylation inhibitor. In some embodiments, the DNA methylation inhibitor is a DNA methyltransferase (DNMT) enzyme inhibitor. Representative DNMT inhibitors including azacitidine (Vidaza®) and decitabine (5 aza 2′ deoxycytidine) (Dacogen®). In some embodiments, the additional anti-cancer agent includes epigenetic therapy. In some embodiments, the epigenetic therapy azacitidine (Vidaza®, Onureg®), decitabine (5 aza 2′ deoxycytidine) (Dacogen®), zebularine (Pyrimidin-2-one β-D-ribofuranoside), guadecitabine, 5-Fluoro-2′dexygctidine, (−)-Epigallocatechin gallate, curcumin, hydralazine, procainamide, RG-108, and SG-1027. See, Nepali et al., J. Biomed. Sci. 28:27 (2021); Giri et al., Front. Pharmacol. 10:1-11 (2019).

### Immunotherapy

In some embodiments, the additional anti-cancer agent includes immunotherapy, e.g., immune checkpoint inhibitors. Representative examples of immune checkpoint molecules that may be targeted by the additional therapy include PD-1, PDL1, CTLA4, KIR, TIGIT, TIM-3, LAG-3, BTLA, VISTA, CD47, and NKG2A. Clinically available examples of immune checkpoint inhibitors include durvalumab (Imfinzi®), atezolizumab (Tecentriq®), and avelumab (Bavencio®). Clinically available examples of PD-1 inhibitors include nivolumab (Opdivo®), pembrolizumab (Keytruda®), and cemiplimab (Libtayo®).

### Chemotherapy

Anti-cancer therapies also include a variety of combination therapies with both chemical and radiation-based treatments. Combination chemotherapies include, for example, Abraxane®, altretamine, docetaxel, Herceptin®, methotrexate, Novantrone®, Zoladex®, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomy emcitabinetabin, mitomycin, etoposide (VP16), tamoxifen, raloxifene, estrogen receptor binding agents, Taxol®, gemcitabine, Navelbine®, farnesyl-protein tansferase inhibitors, transplatinum, 5-fluorouracil, vincristine, vinblastine and methotrexate, or any analog or derivative variant of the foregoing and also combinations thereof.

### Radiotherapy

Anti-cancer therapies also include radiation-based, DNA-damaging treatments. Combination radiotherapies include what are commonly known as gamma-rays, X-rays, and/or the directed delivery of radioisotopes to cancer cells which cause a broad range of damage on DNA, on the replication and repair of DNA, and on the assembly and maintenance of chromosomes. Dosage ranges for radioisotopes vary widely, and depend on the half-life of the isotope, the strength and type of radiation emitted, and the uptake by the neoplastic cells and will be determined by the attending physician.

These and other aspects of the present application will be further appreciated upon consideration of the following Examples, which are intended to illustrate certain embodiments of the application but are not intended to limit its scope, as defined by the claims.

### EXAMPLES

### Example 1: Materials and Methods

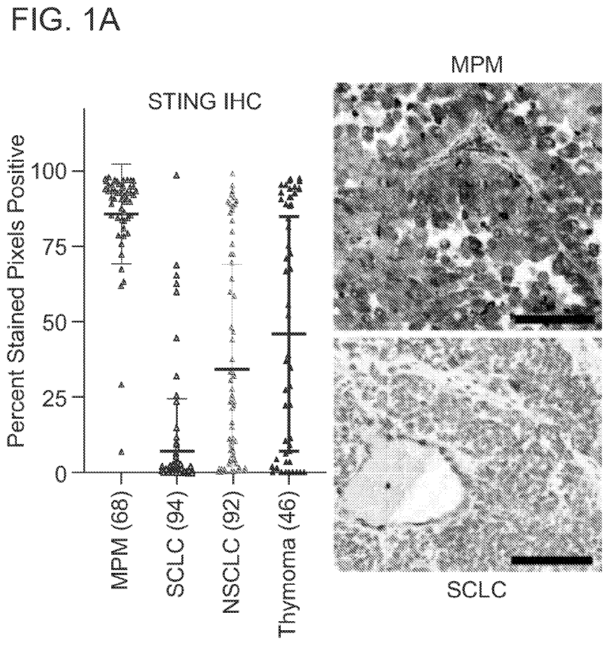

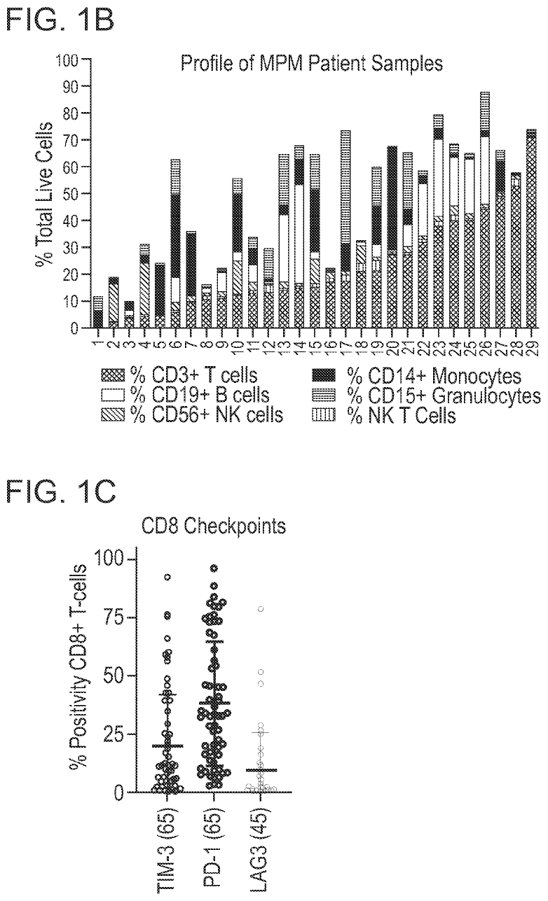

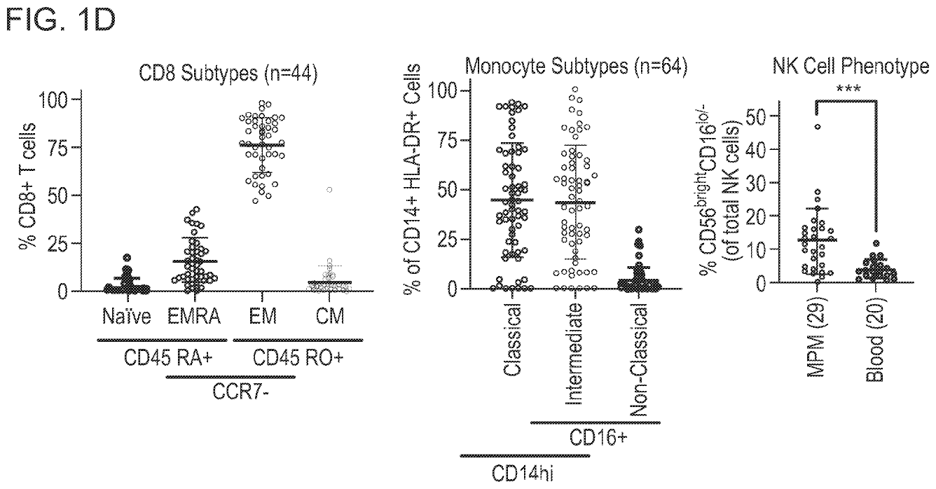

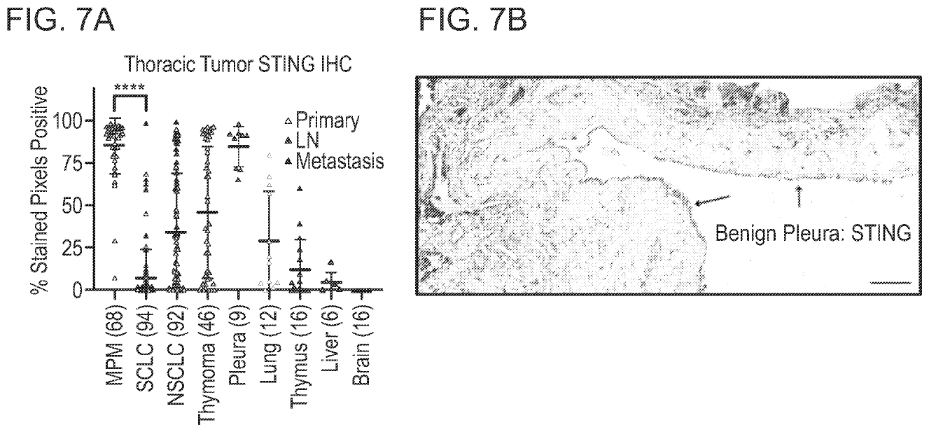

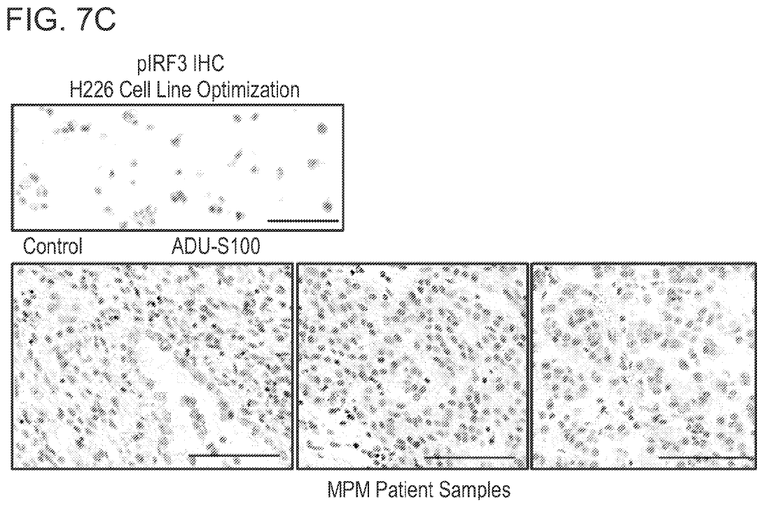

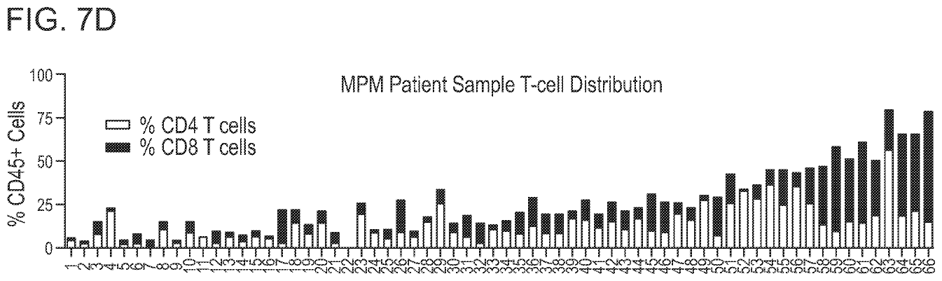

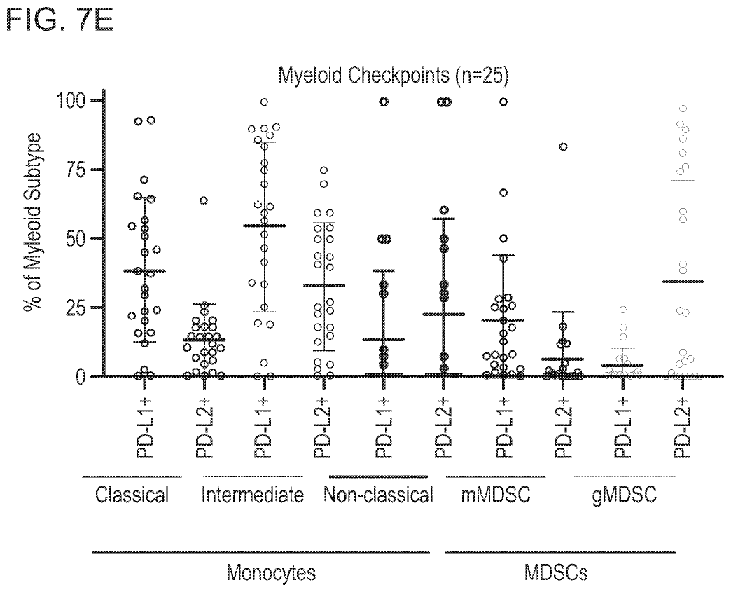

Patient Samples. Formalin-fixed, paraffin-embedded (FFPE) tissue-microarray slides from patients with SCLC, NSCLC, and thymoma were purchased from Biomax (LC245, LC817, LC2081, THY761). In addition, FFPE slides were collected from DFCI/BWH patients with SCLC (n=58), MPM (n=68) and benign pleura (n=9) under Dana-Farber/Harvard Cancer Center protocols 02-180 and 98-063. Tumors from patients with MPM treated at DFCI/BWH between July 2018 and October 2021 were collected after surgery under protocol 98-063. The patient samples analyzed by flow cytometry in FIG. 1A-FIG. 1D and FIG. 7A-FIG. 7E are different than those treated ex vivo in the remaining figures. NK cells were tested in blood collected from patients with head & neck squamous cell carcinoma or oral proliferative verrucous leukoplakia under protocols 17-255 and 18-387 (FIG. 1D). Mann-Whitney test: ***p<0.001. Flow cytometry antibody details provided in Table 1. TIM-3=T-cell immunoglobulin and mucin domain-containing protein 3; PD-1=programmed cell death protein 1; LAG3=lymphocyte activation gene 3; EMRA=effector memory re-expressing CD45 RA; EM=effector memory; CM=central memory. These studies were conducted according to the Declaration of Helsinki and approved by DFCI and BWH institutional review boards. Written informed consent was obtained from all patients whose tumors were studied.

Immunohistochemistry. STING and 25 hosphoro-IRF3 immunohistochemistry (IHC) were performed on the Leica Bond III automated staining platform. The antibody for STING (Cell Signaling Technology #13647, clone D2P2F) was run at 1:50 dilution using the Leica Biosystems Refine Detection Kit with citrate antigen retrieval. The antibody for 25 hosphoro-IRF3 (Cell Signaling Technology #29047, clone D601M) was run at 1:100 dilution using the Leica Biosystems Refine Detection Kit with EDTA antigen retrieval. This was optimized from a range of dilutions and comparison of citrate vs. EDTA antigen retrieval on MPM cell lines treated in vitro with 50 μM ADU-S100 for 24-hours prior to paraformaldehyde fixation and paraffin embedding (FIG. 7C). STING IHC staining was quantified using the QuPath software (version 0.2.3) (Bankhead et al., Sci. Rep. 7:16878 (2017)). Positive Pixel Detection analysis was used with default settings for DAB staining to detect and quantify positive pixels in each of three individual, randomly selected fields per tumor, which were then averaged.

Flow-cytometric immune profiling. Fresh tumors were mechanically and enzymatically disaggregated in dissociation buffer consisting of RPMI (Life Technologies)+10% fetal bovine serum (FBS; HyClone), 100 U/ml collagenase type IV (Life Technologies), and 50 μg/ml dNase I (Roche). The suspension was incubated at 37° C. for 45 minutes and then further mechanically dissociated. Red blood cells were removed from samples using red blood cell lysis buffer (Biolegend). Samples were pelleted and then resuspended in fresh RPMI+10% FBS and strained through a 40 μm filter. Cells were incubated with the Live/Dead Zombie NIR (Biolegend) for 5 minutes in the dark at room temperature. Fc receptors were blocked prior to surface antibody staining using Human TruStain FcX Blocking Reagent (Biolegend). Cells were stained for 15 minutes on ice in the dark and washed 2× with PBS+2% FBS. Cells were analyzed on a BD LSRFortessa with FACSDiva software (BD Biosciences). Data were analyzed using FlowJo software version 10.5.3. Antibodies are listed as protein target with clone, manufacturer and catelog number in paraenetess, CD69 (FN50, BioLegend, 310904), CD16 (3G8, BioLegend, 302006), CD8 (RPA-T8, Thermo Fisher, BDB560662), CCR2 (K036C2, Biolegend, 357203), CD38 (HIT2, BioLegend, 303506), CD11c (3.9, BioLegend, 301605), CCR7 (150503, Thermo Fisher, BDB62381), CD56 (GDC56, BioLegend, 318348), LAG-3 (11C3C65, BioLegend, 369309), CD103 (B-Ly7, Thermo Fisher, 25-1038-41), TIM-3 (F38-2E2, BioLegend, 345012), PD-L1 (29E.2A3, BioLegend, 329708), CD3 (UCHT1, BioLegend, 300424), PD-1 (EH12.2H7, BioLegend, 329920), HLA-DR (G46-6, Thermo Fisher, BDB562804), CD45RA (HL100, BioLegend, 304142), CD15 (SSEA-1, BioLegend, 323028), CTLA-4 (BNI3, BioLegend, 369609), CD19 (HIB 19, BioLegend, 302243), CD45 (H130, BioLegend, 304050), CD4 (PRA-T4, BioLegend, 300554), CD14 (M5E2, BioLegend, 301840), Mesothelin (REA1057, Miltenyi, 130-118-168), STING (D2P2F, Cell Signaling, 13647), pTBK1 (D52C2, Cell Signaling, 5483), TBK1 (Polyclonal, Cell Signaling, 3013), pIRF3 (4D4G, Cell Signaling, 4947), IRF3 (D6I4C, Cell Signaling, 11904), pSTATI (58D6, Cell Signaling, 9167), STAT1 (Polyclonal, Cell Signaling, 9172), IFNAR-1 (Polyclonal, Thermal Fischer, PA5-79441), and -Actin (C4, Santa Cruz, sc-47778).

Patient-derived organotypic tumor spheroids (PDOTS). PDOTS were generated as previously described (Jenkins et al., Cancer Discov. 8:196-215 (2018); Aref et al., Lab Chip 18:3129-3143 (2018)). Briefly, fresh tumor specimens were minced in a 15 mL falcon tube in prewarmed to 37° C. full media (DMEM from Thermo Fisher Scientific+10% FBS)+100 U/mL collagenase type IV (Life Technologies) and 50 μg/mL dNase I (Roche) for approximately 20 minutes using sterile scissors and pipetting. Dissociated material was strained over 100-μm filter and 40-μm filters to generate S1 (>100 μm), S2 (40-100 μm), and S3 (<40 μm) spheroid fractions, which were subsequently maintained in ultralow-attachment (ULA) tissue culture plates (Corning). S1 fractions were treated with 50 μM ADU-S100 (Chemietek) for cytokine analysis and single-cell RNA sequencing. S2 fractions were used for ex vivo culture by resuspending them in type I rat tail collagen (Corning) at a concentration of 2.8 mg/mL prior to loading into the center gel region of the 3-D microfluidic culture device (AIM Biotech) and incubation for 40 minutes at 37° C. in humidity chambers to allow for polymerization. Collagen hydrogels containing PDOTS were hydrated with media with or without indicated treatments. TAK-676 was provided by Takeda and diluted in dH20. Recombinant human interferon beta (100 ng/ml; R&D Systems) was used as a positive control downstream of STING for STAT1 pathway activation. CD8a was neutralized with 50 μg/mL InVivoMAb antibody vs. IgG control (BE0092). CXCR3 was neutralized with 5 μg/mL human CXCR3 antibody (R&D MAB160).

PDOTS immunofluorescence and live/dead quantification. Dual labeling was performed by loading microfluidic devices with Nexcelom ViaStain acridine orange/propidium iodide (AO/PI) Staining Solution (Nexcelom, CS2-0106) or 10 μg/mL solution of Hoechst 33342 (Thermo Fisher Scientific) and 1 μg/mL solution of PI (Thermo Fisher Scientific). Following incubation with the dyes (20 minutes at room temperature in the dark for AO/PI or 45 minutes for Hoechst 33342/PI), images were captured using 4× objective of a Nikon Eclipse 80i fluorescence microscope equipped with automated motorized stage (Proscan), Z-stack (Prior), and Zyla 5.5 sCMOS camera (Andor). Image capture and analysis were performed using NIS-Elements AR software package. Live and dead cell quantitation was performed by measuring total cell area of each dye. For additional immunofluorescence studies, PDOTS were washed with PBS and blocked with FcR blocking reagent (Miltenyi) for 30 minutes at room temperature. Directly conjugated antibodies CD326 EpCAM-AlexaFluor647 (clone 9C4), CD45-AlexaFluor647 (HI30) (BioLegend), and mesothelin-PE (clone REA1057, Miltenyi) were diluted 1:50 in 10 μg/mL solution of Hoechst 33342 (Thermo Fisher Scientific) in PBS and loaded into microfluidic devices for 1-hour incubation at room temperature in the dark. Spheroids were washed twice with PBS with 0.1% Tween20 followed by PBS. For viability assessment, microfluidic devices were loaded with 1:1,000 solution of calcein AM (Thermo Fisher Scientific) in PBS. For IRF3 IF, PDOTS were treated for 3 hours with dH20 control or 50 μM ADU-S100, washed with PBS, fixed with 4% paraformaldehyde for 15 minutes, and permeabilized with 0.1% Triton-X for 10 minutes. Cell Signaling Antibody #11904 (clone D614C) was diluted 1:50 in PBS and incubated for 45 minutes, washed, and subsequently incubated in FITC-conjugated anti-rabbit secondary antibody (Thermo Fisher Scientific) diluted 1:100 for 30 minutes. PDOTS were washed twice with PBS with 0.1% Tween20 and counterstained with 1 μg/mL solution of Hoechst 33342. Images were captured as mentioned above for live/dead dual staining, using a 20× objective.

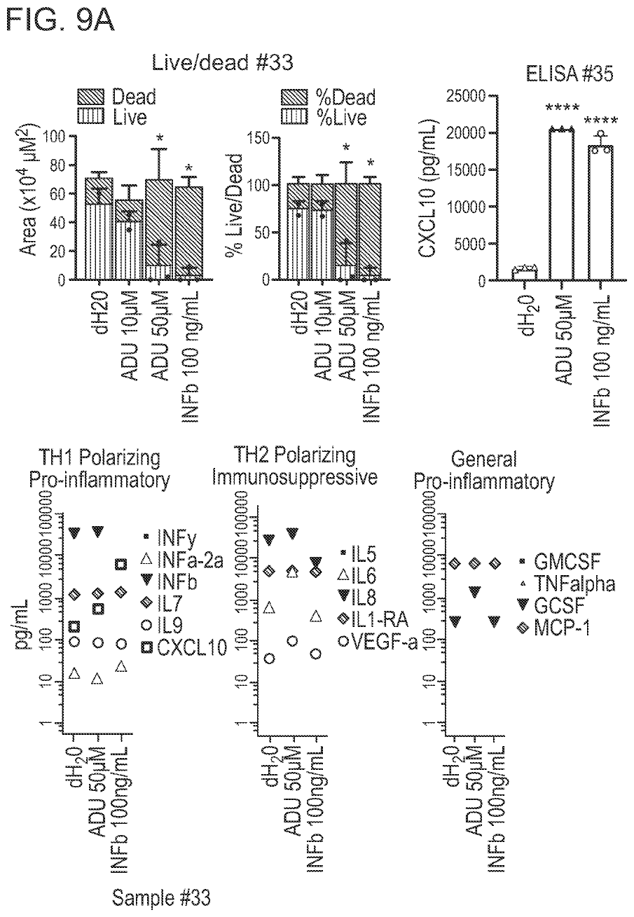

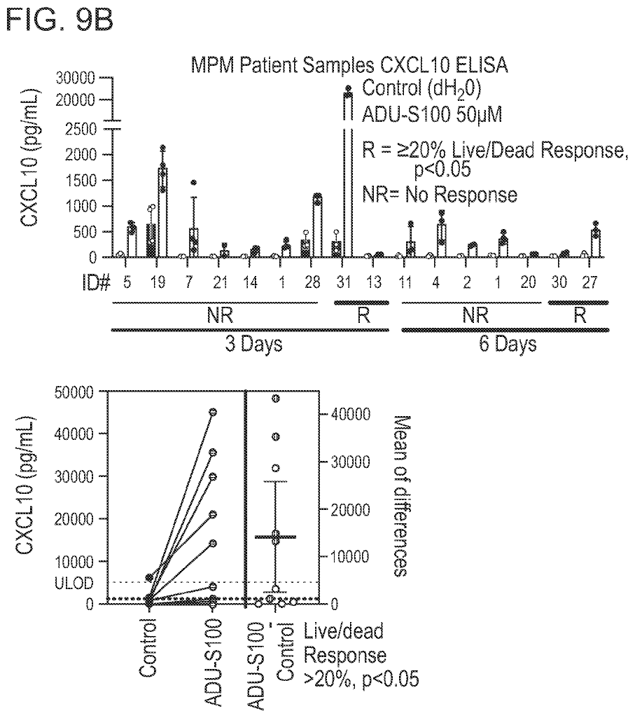

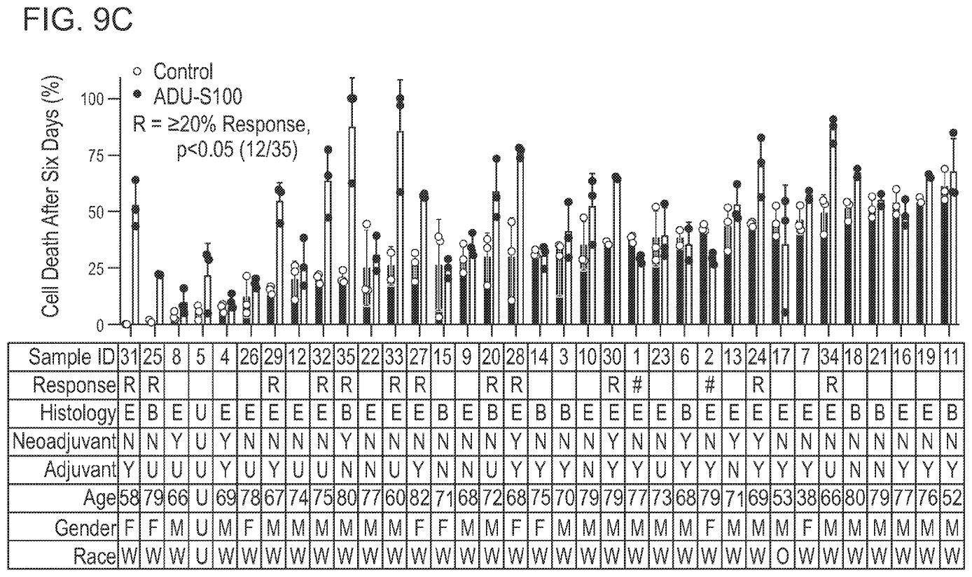

Cytokine analysis. CXCL10 ELISA (R&D Systems DIP100) and granzyme B ELISA (R&D systems DY008) were performed according to manufacturer's instructions on conditioned media collected from cell culture. Cytokine analysis of conditioned media after 3 days of explant (S1) culture (FIG. 9B) utilized the MSD U-PLEX Viral Combo 1 assay (Hu: K15343K-2), which was performed according to manufacturer's instructions.

2′3′ cGAMP ELISA. Cayman Chemical 2′3′ cGAMP ELISA kit was performed according to manufacturer's instructions to detect levels of 2′3′ cGAMP in the supernatant of the MPM cell lines. For these experiments, 3-5×10⁵ cells were plated in a 6-well plate and transfected using X-tremeGENE HP DNA Transfection Reagent combined with Opti-MEM Reduced-Serum media and 1 μg poly (dG:dC) (Invivogen) for a 30-minute incubation. 2′3′ cGAMP (Invivogen) was used as a positive control.

Cell culture. MPM cell lines were cultured in RPMI-1640 (Thermo Fisher Scientific) supplemented with 10% FBS (Gemini Bio-products). H226, H28, MSTO-211H, H2452 and H2052 were purchased from ATCC. MS428 was provided by the Richards Lab. H2461 and H2591 were provided to Dr. Janne by the NIH (Pass et al., Ann. Thorac. Surg. 59:835-44 (1995)). JMN1B (Demetri et al., Blood 74:940-6 (1989)) and MS589 (Gordon et al., Am. J. Pathol. 166:1827-40 (2005)) were derived at BWH/DFCI and shared internally with permission. All experiments were performed before reaching 10 passages. Mycoplasma infection was regularly checked by PCR using the conditioned media derived from each cell line with primers as previously described (Kitajima et al., Cancer Discov. 9:34-45 (2019)).

Immunoblotting. Cells were lysed in RIPA buffer containing 1× protease inhibitors (Roche 11-836-145-001) and phosphatase inhibitors (50 mmol/L NaF and 100 mmol/L Na3VO4). Immunoblotting was performed as previously described (Kitajima et al., Cancer Discov. 9:34-45 (2019)) using the antibodies listed in Table 1. Secondary antibodies were from LI-COR Biosciences: IRDye 680LT Goat anti-Mouse IgG (#926-68020) and IRDye 800CW Goat anti-Rabbit IgG (#926-32211). Imaging of blots and was performed using the LI-COR Odyssey system.

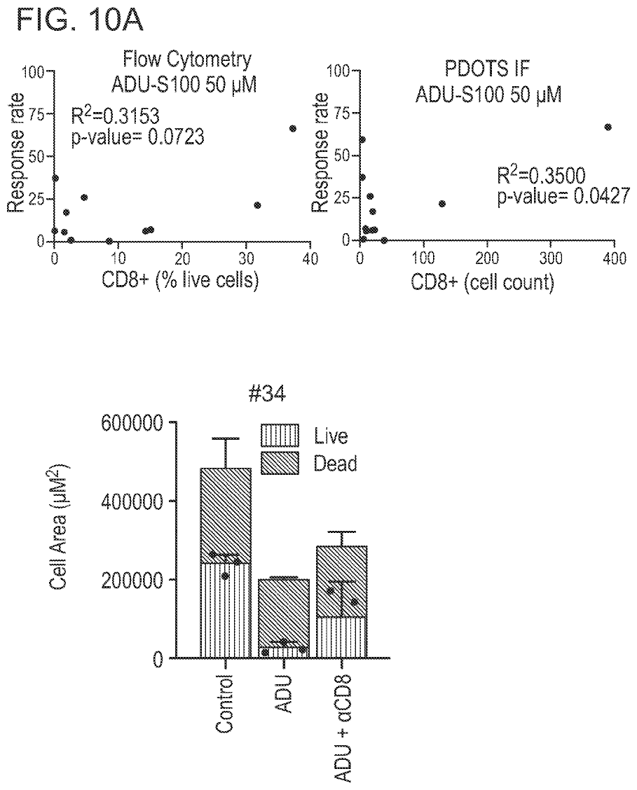

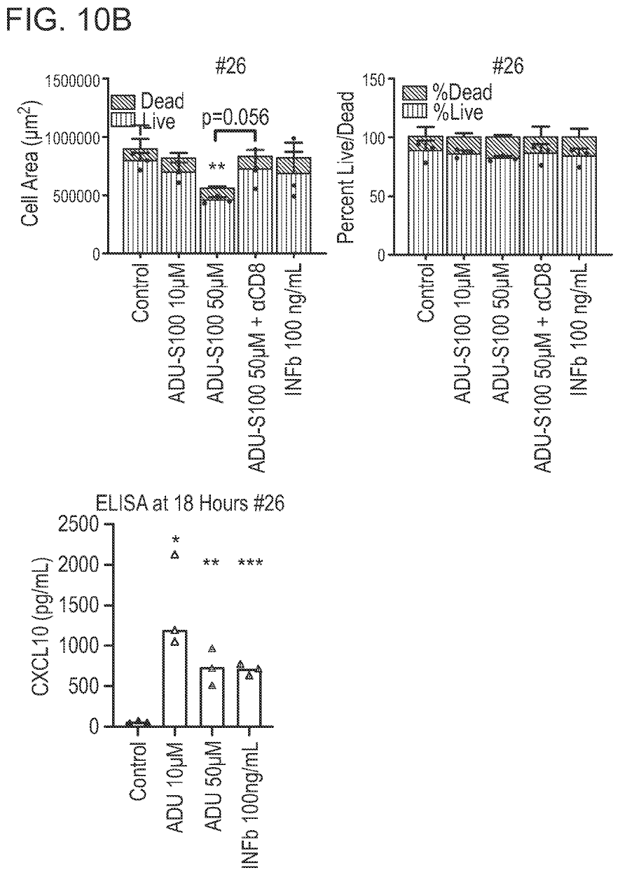

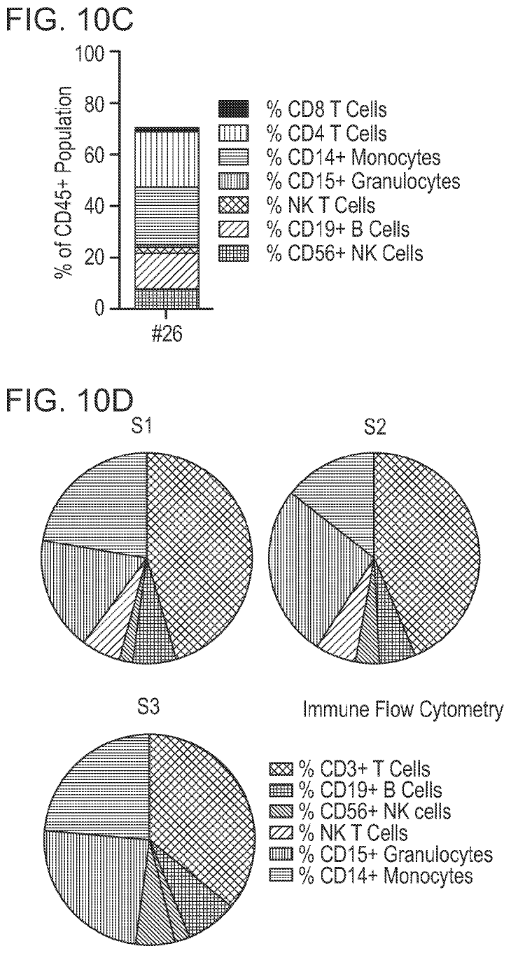

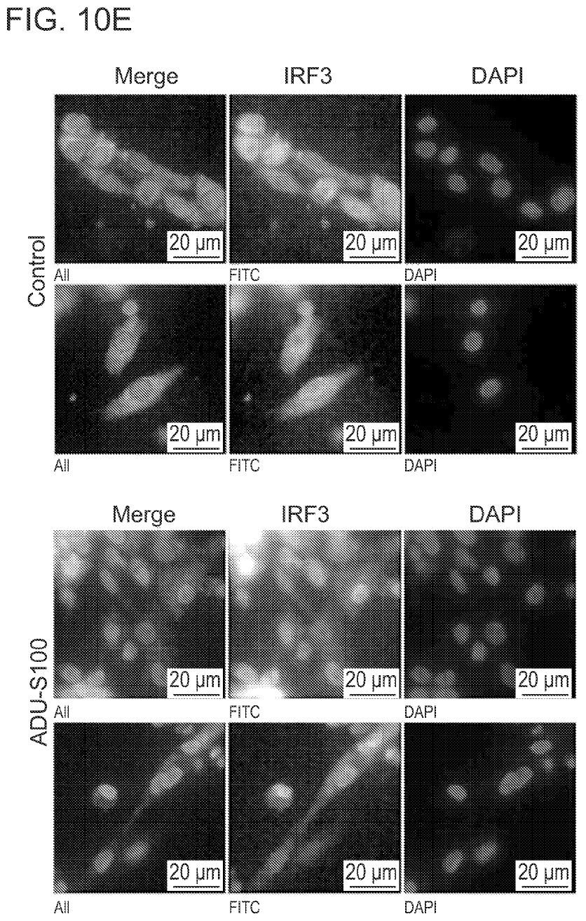

Dynamic single-cell RNA sequencing and data analysis. The previous protocol (Sehgal et al., J. Clin. Invest. 131:e135038 (2021)) was adapted to test S1 explants from MPM PDOTS. The sample tested (#26) demonstrated baseline viability of 63% and 18-hour cytokine release in response to treatment (FIG. 10B). After 24 hours of treatment in a ULA dish, tumor spheroids were digested with trypsin in a 37° C. incubator for 5 minutes to obtain single-cell suspensions. Cells were loaded onto a 10× chromium instrument (10× Genomics) per manufacturer's instructions. ScRNA libraries were generated using the single cell 3′ reagent kit (10× Genomics) per the user guide. Quality control of the completed libraries was performed using a bioanalyzer high sensitivity DNA kit (Agilent) and then sequenced using the Illumina NextSeq 500 platform.

Raw sequencing reads were processed using the 10× Genomics CellRanger bioinformatics pipeline v6.0.1. The assembled matrix was then fed into the standard workflow of the R package, Seurat v4.0.4. Genes that were expressed in at least 3 cells, and only cells that expressed at least 2 genes, were kept for downstream processing. Additionally, cells expressing more than 7000 genes and cells with more than 10% of UMIs mapping to mitochondrial genes were removed from the analysis. All the samples were prepared and sequenced together on the same platform. The filtered matrix was log-normalized using global scaling in Seurat. UMI and mitochondrial transcript content were used as regression parameters. The normalized matrix was scaled and centered gene-wise, and then underwent dimensionality reduction using principal component analysis (PCA) on the highly varying genes. After visual inspection of the PCA elbow plot, the top 10 PCs were chosen for further analysis. Clustering was performed on the chosen PCs using the shared nearest neighbor algorithm in Seurat with default parameters.

A Uniform Manifold Approximation and Projection (UMAP) map was computed and plotted with the DimPlot module of Seurat. Cluster differential expression analysis was performed in Seurat using the FindMarkers command using the Wilcoxon rank sum test without thresholds. Contour plots overlayed onto UMAPs were generated with R package ggplot2 (Wickham, Springer-Verlag New York (2016)).

Cell types were identified based on comparative analysis of the signatures published previously (Han et al., Cell 172:1091-1107 (2018); Muhl et al., Nat. Commun. 11:3953 (2020); Correia et al., Proc. Natl. Acad. Sci. U.S.A. 115:E5980-E5989 (2018); Gueugnon et al., Am. J. Pathol. 178:1033-42 (2011)), as well as marker genes identified in this study, which were used to remove genes ubiquitously expressed across cell subpopulations. Collagen-encoding genes were added to the fibroblast signature. The list of gene signatures used for enrichment analysis in provided in Table 1.

TABLE 1
Gene signatures for scRNAseq
B cell
CD79A, IGHM, CD79B, MS4A1
Macrophage
CD14, VSIG4, C1QB, C1QA, APOE
Fibroblast
COL3A1, COL4A1, COL4A2, COL4A5, COL4A6, COL5A1, COL5A2, COL6A1, COL6A2,
COL8A1, COL12A1, COL17A1, S100A16, UGDH, LOXL1, PCOLCE2, ADAMTS2
T cell
TCF7, CD3G, CD3D, CD4, CD28, BCL11B, CD8A, CD8B
Tumor
MSLN, ANXA8, CALB2, INHBA, ITLN1, MGARP, HEG1
NK cell
SH2D1B, TRDC, TYROBP, KLRD1, TNFRSF18, NKG7, KLRB1, GNLY, IL2RB, CTSW,
ALOX5AP, GZMB
NK Active
PRF1, KLRB1, TNFSF10, CCL5, NKG7, KLRD1, NCAM1, NCR1, NCR2, NCR3, FCER1G,
KIR2DL1, KIR2DL3, KIR2DL4, KLRC1, KLRC4, FCGR3A, KIR3DL1, KIR3DL2
NK T cell
CD3D, CD3E, CD2, CCL5, CST7, TRBC2, CXCR4, CD8A, GZMK, IL32, PLAAT4, GZMA
Other T cell
SELL, CCR7, S1PR1, SPOCK2, GIMAP5, GIMAP7, LTB, CXCR4, IL7R, YNE2, SARAF,
IFITM1
V-gene
TRBV2, TRBV3-1, TRBV4-1, TRBV5-1, TRBV6-1, TRBV4-2, TRBV6-2, TRBV7-2,
TRBV6-4, TRBV7-3, TRBV9, TRBV11-2, TRBV6-5, TRBV7-4, TRBV5-4, TRBV7-6,
TRBV7-9, TRBV13, TRBV10-3, TRBV12-3, TRBV12-4, TRBV14, TRBV18, TRBV19,
TRBV20-1, TRBV21-1, TRBV24-1, TRBV27, TRBV28, TRBV29-1, TRBV30, TRAV1-2,
TRAV2, TRAV4, TRAV5, TRAV6, TRAV8-1, TRAV10, TRAV12-1, TRAV8-2, TRAV8-3,
TRAV13-1, TRAV12-2, TRAV8-4, TRAV13-2, TRAV14DV4, TRAV9-2, TRAV12-3,
TRAV8-6, TRAV17, TRAV19, TRAV21, TRAV22, TRAV23DV6, TRAV24, TRAV25,
TRAV26-1, TRAV29DV5, TRAV26-2, TRAV35, TRAV36DV7, TRAV38-1, TRAV38-
2DV8, TRAV39, TRAV41

Isolation of tumor-infiltrating lymphocytes. TILs were isolated from patient specimens under IRB protocol 02-180 and filtered as described above for PDOTS. The S3 fraction was expanded using RPMI-1640 with L-glutamine, 1% Penicillin-Streptomycin solution, 1 mM Na Pyruvate, 0.0375% Na Bicarbonate, 50 nM mercaptoethanol, 10% Human AB Serum and 6000U/mL IL-2 in a 24-well plate and split 1:2 every other day over a period of 8-10 days. Upon expansion they were frozen/stored in liquid nitrogen.

Expansion and transduction of primary T-cells. Peripheral blood mononuclear cells (PBMCs) were obtained from healthy donors after informed consent and isolated by using Ficoll density centrifugation. Isolated PBMCs were activated with TransAct (1:100, Miltenyi) in complete medium (RPMI-1640 supplemented with 10% FBS, in the presence of IL-2 at 10 ng/ml). Two days after activation, the T-cells were lentivirally transduced by spinoculation with the BCMA CAR virus (1% virus volume) in the presence of Lentiboost (1:100, Sirion Biotech). The BCMA CAR sequence has been previously described (Works et al., Mol. Cancer Ther. 18:2246-2257 (2019)) and was cloned into the pHAGE lentiviral vector (Addgene plasmid #24526) and the generated plasmid was subjected to sequencing verification. For packaging and production of lentivirus particles, 293 Lenti-X packaging cells (Takara) were seeded into a 15 cm plate (8×10⁶ cells/plate) for 24 h, then transfected with plasmids encoding CAR (pHIV-aMeso-CAR), pMD.2 G encoding VSV-G envelope, and a packaging vector psPAX2 using PEI transfection reagent (Polysciences). Virus supernatants were harvested at 24 hours and 48 hours after transfection, filtered through a 0.45 μm membrane, and concentrated by ultracentrifugation and stored at −80° C. prior to transduction. After transduction, T-cells were expanded with cytokines, IL-2 (10 ng/ml), IL-7 (3 ng/ml), and IL-15 (10 ng/ml), in RPMI-1640 supplemented 10% FBS, and their transduction efficiency was determined by FACS three days after transduction.

Expansion and transduction of primary NK cells. For experiments using unmanipulated primary NK cells, CD56+CD3− NK cells were expanded from human PBMCs (Lonza) using the CellXVivo Human NK Cell Expansion Kit (R&D Systems). Following 14 days of expansion, cells were transitioned to culture in CTS OpTmizer T-cell expansion media supplemented with 5% human AB serum (Sigma Aldrich), 1% GlutaMAX, 1% HEPES, and 1% Penicillin-Streptomycin in the presence of IL-2 (PeproTech or Miltenyi; 200 U/mL for flow cytometry experiments, 500 U/mL for killing experiments including PDOTS). All NK cell culture reagents were purchased from Life Technologies unless otherwise stated.

For experiments using transduced and control-processed primary NK cells, they were extracted from whole blood leukapheresis using RosetteSep (StemCell technologies) and Ficoll-Paque density gradient centrifugation under the approved Crimson Study protocol T0197. The isolated NK cells were inspected for purity and cultured for 2 days in RPMI (Gibco) supplemented with 10% heat-inactivated (HI)-FBS (Gibco), 1% Penicillin-Streptomycin, 2 mM L-Glutamine and HEPES in the presence of IL-15 (1 ng/ml; Miltenyi). Isolated NK cells were subsequently transduced as below or cultured in NK MACs media (Miltenyi) supplemented with 5% human serum (Sigma) and 1% v/v Penicillin-Streptomycin (Gemini Bio-products) in the presence of IL-2 (500 U/mL; Miltenyi).

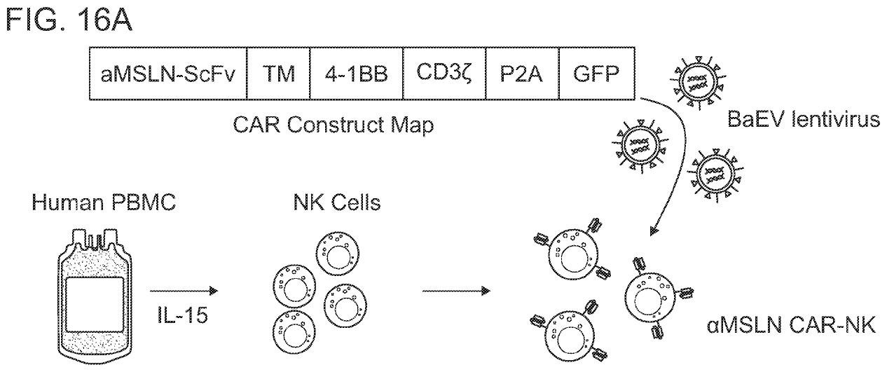

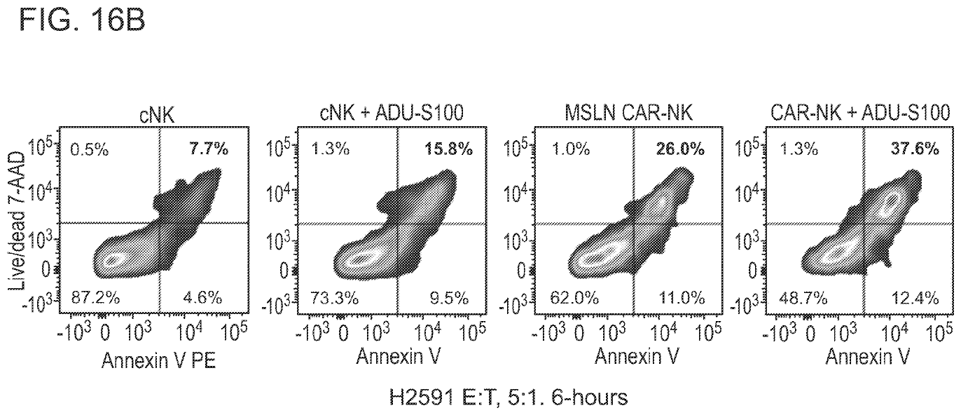

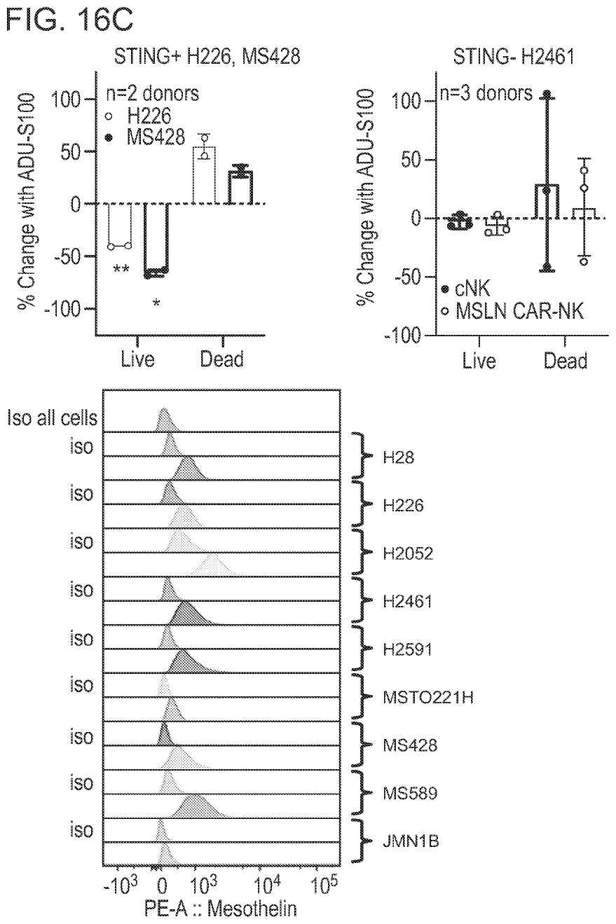

CAR Constructs. CAR constructs were designed with extracellular ScFv domain, transmembrane segment derived from the CD8 protein. This is followed by traditional 4-1BB and CD3 co-stimulatory domains. The CAR gene is designed to incorporate an HA tag for analysis using flow cytometry. For the gene constructs with CXCR3, the CAR gene was followed by P2A self-cleaving peptide nucleic acid and a CXCR3. To generate CAR or CAR-CXCR3 NK cells, primary NK cells were purified from peripheral blood, activated using IL-12, IL-15, and IL-18 which results in the activation and differentiation of NK cells to generate cytokine-induced memory-like (CIML). Conventional NK cells (cNK) were used as control which were maintained at low dose IL-15 (1 ng/mL). The CAR gene was transduced into cNK or CIML NK cells via our optimized baboon lentiviral system to achieve high transduction efficiency. Anti-Mesothelin CAR (aMSLN) was constructed in a pHIV backbone, as illustrated in FIG. 30A with the mesothelin specific ScFv derived from YP218 antibody, followed by transmembrane domain and co-stimulatory domains (4-1BB and CD3). The construct also contains EGFP fragment separated from the CAR fragment by self-cleaving P2A (FIG. 16A). The CAR gene construct was packaged into BaEV-pseudotyped lentiviral system by transfecting HEK-293 cells with pCMV-BaEV, pCMV-A8.9 and pAdv plasmids. The viral particles were titrated using Jurkat cells. Assuming a multiplicity of infection (MOI) of 1 for Jurkat cells, the viral titers were calculated to transduce NK cells with MOI of 10. NK cells were transduced using Retronectin and vectofusin followed by spinfection+/−active lentivirus (cNK control without virus) two days after extraction and subsequently cultured in NK MACs media (Miltenyi) supplemented with 5% human serum and 1% Penicillin-Streptomycin (Gemini Bio-products) in the presence of IL-2 (500 U/mL; Miltenyi). The percentage of NK cells expressing CAR was determined via flow cytometric analysis of GFP and surface expression of ScFv using APC Human agglutinin (HA).

Immune Cell Toxicity Assays. For flow cytometry immune cell toxicity assays, primary NK cells and TILs were seeded at 200,000 cells per well (NK or TILs alone or 1:1 with 100,000 cells of each type) in 96-well plate alone or in co-culture and treated with 10 UM or 50 M ADU-S100 (Chemietek) or dH20 control with or without IL-2 (Miltenyi or PeproTech) at the indicated concentrations for 72 hours. Following treatment, samples were stained with anti-CD45, anti-CD3, anti-CD4, anti-CD8, and anti-CD56 antibodies (Table 1), as well as Zombie Green live/dead (Biolegend 423111) and analyzed by flow cytometry as described above. Data were analyzed using FlowJo software version 10.5.3. As an orthogonal measure of viability, CellTiter-Glo was performed on primary T-cells and NK cells.

CellTiter-Glo luminescent cell viability assay. Cell viability was assessed using the CellTiter-Glo Luminescent Cell Viability assay (Promega, G7571) according to manufacturer's instructions. For untransduced primary T-cells and BCMA CAR T-cells, 25,000 cells per well were seeded in 96-well plate and treated with ADU-S100 or dH20 as control for 24 hours at the indicated concentrations. For NK cells, 25,000 cells per well were seeded and treated with ADU-S100 or dH20 as control for 24 hours at the indicated concentrations. For MPM cell lines, 10,000 cells per well (MS428) or 12,500 cells per well (H2461, H2591) were seeded in 96-well plate and treated with ADU-S100 50 μM or media as control for indicated times. All conditions were tested in triplicate and plates were read on a Tecan Infinite Mplex Microplate Reader.

Autophagy Staining. Autophagy was assessed by vacuole staining to identify autophagolysosomes using the CYTO-ID Autophagy detection kit 2.0 (Enzo ENZ-51031-0050) according to manufacturer's instructions. Briefly, 5×10⁵ isolated primary NK cells or TILs were incubated in T-cell growth media (TCGM) with 500 U/mL IL-2, which was refreshed every time the media was changed to ensure proper growth and selection. CLQ from the kit (Enzo 51005-CLQ) was used starting at the recommended initial dose of 10 μM compared with DMSO control. After 24 h the media was changed, and the cells were treated for another 24 h with CLQ+10 μM ADU-S100. The media was collected, and flow cytometry was performed after staining following manufacturer's instructions with CYTO-ID Green Detection Reagent 2.

NK cell killing assay. Target cells (MPM cell lines) were detached via trypsinization, labelled with CellTrace Violet (CTV, LifeTechnologies) and then seeded in a 96-well plate at a cell density of 25,000 cells per well. Target cells were allowed to adhere for 12-16 hours, and NK or aMSLN-CAR-NK cells were then added at different effector to target (E:T) ratios (1:1, 2:1, 5:1 and 10:1) with or without ADU-S100 (50 μM). After 6 hours of co-culture, the cells were harvested and incubated with an antibody for the apoptosis marker Annexin V (PE) and the live/dead stain 7-AAD (Biolegend). Cells were analyzed on a BD LSRFortessa with FACSDiva software (BD Biosciences). Data were analyzed using FlowJo software version 10.5.3. The apoptotic cells were evaluated by gating on the CTV+ population and represented as percentage live or dead (late apoptotic) cells. Apoptotic cell analysis was conducted using NK cells extracted from as many as 4 different healthy donors per target MPM cell line to incorporate baseline donor variability.

NK cell infiltration assay. Immune cell infiltration was assessed as previously described (Kitajima et al., Cancer Discov. 9:34-45 (2019); Mahadevan et al., Cancer Discov. 11:1952-1969 (2021)). Briefly, mesothelioma cancer cell spheroids (H2591, H2461, H226) were generated by seeding 5×10⁵ cells in suspension in a ULA dish for 24 hours. H226 cells were treated with 50 μM ADU-S100 during the final 6 hours of spheroid formation to establish a cytokine gradient. Samples were then pelleted and resuspended in type I rat tail collagen (Corning) at a concentration of 2.5 mg/mL following the addition of 10×PBS with phenol red and pH adjustment using NaOH. pH 7.0-7.5 was confirmed using PANPEHA Whatman paper (Sigma-Aldrich). Cells and collagen were kept on ice to prevent polymerization. The spheroid-collagen suspension was then injected into the central gel region of the 3D DAX-1 microfluidic cell culture chip (AIM Biotech). Microfluidic devices were utilized as previously described (Aref et al., Lab Chip 18:3129-3143 (2018)), with a central region containing the cell-collagen mixture in a 3D microenvironment (3×10⁴ cells H2591 and H2461, 2× 10⁴ cells H226 in 10 μL), flanked by 2 media channels. After injection, collagen hydrogels containing cells were incubated for 40 minutes at 37° C. in humidity chambers, then hydrated with culture media, with labeled primary NK cells (ET ratio 2:1) added to one of the side channels. Primary NK cells were labeled with Cell Tracker Red (Thermo Fisher Scientific) following manufacturer's instructions. After 96 hours of incubation, viability staining of cancer cell spheroids and infiltrated immune cells was performed (20-minute incubation with 1 μg/mL solution of Propidium Iodide; Thermo Fisher Scientific). For the experiment with the CXCR3 neutralizing antibody (R&D MAB160), NK cells were pre-treated for 30 minutes prior to loading.

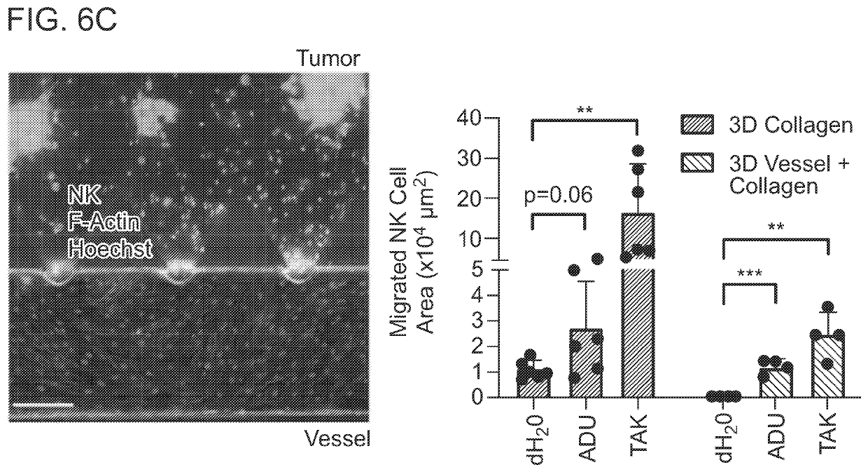

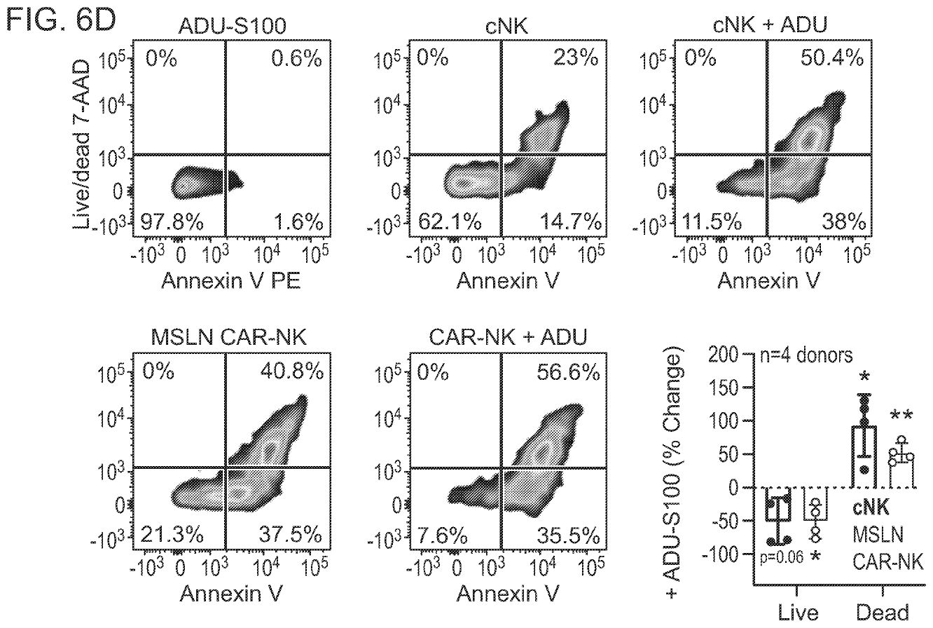

For quantification, images were captured on a Nikon Eclipse 80i fluorescence microscope equipped with Z-stack (Prior) and CoolSNAP CCD camera (Roper Scientific). Image capture and analysis was performed using NIS-Elements AR software package. Whole device images were achieved by stitching in multiple captures. Quantification of immune cell infiltration into the 3D tumor microenvironment was performed by measuring the total cell area of cell tracker dye in the entire gel region. For the experiment with the CXCR3 neutralizing antibody (R&D MAB160; FIG. 6B), staining was quantified in a square region in the center of the channel to focus on effects of CXCR3 ligands released by tumor cells. Percent dead cell quantification was performed as in PDOTS as described above.

3D vascular model. To generate the tumor-vascular model, H226 spheroids were mixed with collagen rat tail hydrogel (2.5 mg/ml) and injected into the center gel region of the 3D microfluidic chamber (10-15 μL per each microfluidic chamber). After incubation for 30 minutes at 37° C. in sterile humidity chambers, the side wall of one flanked channel (media channel) was coated with a 150 μg/ml collagen solution in PBS to allow for better adhesion of eCs to the channel. After 15 mins, the channel was washed once with media. To create the 3D vessel, 25 μL cell suspension of 3×10⁶ cells/ml human umbilical vein endothelial cells (HUVECs; C2519AS, Lonza) were injected in the media channel coated with collagen. The channel was rotated twice to create a confluent hollow-lumen 3D vessel. To allow the cells to attach to the media-gel interface and form a monolayer, the chip was incubated with cells face down for 15 mins. Next, 50 μL cell suspension was reinjected, and the chip was flipped to cover the upper part of the 3D vascular channel. After 90 mins of incubation in the humidity chamber at 37° C., cell culture media was gently added to both channels and further incubated to form a confluent monolayer. After vessel formation, NK cells (labelled with cell tracker) were added to the 3D vessel at 2:1 E:T ratio. Treatment with STING agonists (ADU-S100, TAK-676) was added to the fluidic channel opposite the vascular barrier. NK cell migration+/−vessel was quantified at 24 hours. Image capture and analysis was performed using a fluorescence confocal microscope and processing software. The 3D vascular channels were rinsed in PBS and fixed with 4% PFA for 15 min at room temperature. Cell membranes were permeabilized with 0.1% Triton X-100 for 5 min at room temperature and washed twice with PBS. HUVEC cells were stained for F-actin with green phalloidin (Thermo Fisher Scientific A12379) and Hoechst 33342. Images were captured on a Nikon Eclipse 80i fluorescence microscope equipped with Z-stack (Prior) and CoolSNAP CCD camera (Roper Scientific).

Immune cell migration assays were performed as previously described (Kitajima et al., Cancer Discov. 9:34-45 (2019)). Briefly, NCI-H226 cells were plated at a density of 5×10⁵ cells per well of a 6-well plate and using treated with STING agonist (ADU-S100) for 24 hours at 50 μM. Spheroids were generated by seeding 5×10⁵ ultra-low attachment dish for 24 hours and were labelled with a fluorescent dye (cell proliferation dye eFluor 450, Invitrogen, 65-0842) per the manufacturer's instructions. Spheroids were pelleted and then resuspended in type I rat tail collagen (Corning) at a final concentration of 2.5 mg/mL following the addition of 10×PBS containing phenol red on ice. The pH of the resulting spheroid suspension was adjusted to 7.0-7.5 using NaOH and confirmed using PANPEHA Whatman paper (Sigma-Aldrich). The spheroid-collagen suspension was then introduced into the central channel of the 3-D microfluidic cell culture chamber (AIM Biotech, design previously described (Aref et al., Lab Chip 18:3129-3143 (2018)). Collagen hydrogels containing cancer cell spheroids were incubated for 40 min at 37° C. in humidity chambers, following which, RPMI-1640 media containing NK cells at an effector-to-target (E:T) ratio of 2:1, was perfused through one of the side channels located next to the central channel. The cancer cell spheroids and NK cells were co-cultured for 3 days, following which NK cell migration into the collagen hydrogel was visualized through images captured on a Nikon Eclipse 80i fluorescence microscope equipped with Z-stack (Prior) and CoolSNAP CCD camera (Roper Scientific), and analyzed using NIS-Elements AR software package. Quantification of immune cell infiltration into the central channel was performed by measuring the total area occupied by the Cell Tracker Red dye-positive cells located in regions of interest (ROI; 6 ROI/microfluidic cell culture chamber).

Statistical analysis. Statistical significance was assessed using unpaired two-tailed Student t-test for pairwise comparisons, one-sample t-test against an expected value of 0% change or 100% control, or one-way ANOVA followed by Tukey post hoc test. Kruskal Wallis global test followed by Dunn's multiple comparisons post hoc test was used for non-parametric analysis of IHC scores and mRNA expression in MPM cell lines (data obtained from the Cancer Cell Line Encyclopedia at the Broad Institute). P values less than 0.05 were considered significant. Asterisks used to indicate significance correspond with *p<0.05, **p<0.01, ***p<0.001, ****p<0.0001. Columns represent means±SD. In one-way ANOVA followed by post hoc tests, asterisks are shown only in pairs of items of interest. GraphPad Prism (version 9.2.0) was used for all statistical analysis.

### Example 2: STING is Primed for Activation in Malignant Pleural Mesothelioma (MPM)

Described herein, is that finding that malignant pleural mesothelioma robustly expresses tumor cell STING and is responsive to STING agonist treatment ex vivo. Dynamic single-cell RNA sequencing of explants treated with a STING agonist unveiled CXCR3 chemokine activation primarily in tumor cells and cancer associated fibroblasts, as well as T-cell cytotoxicity. In contrast, primary NK cells resisted STING agonist-induced cytotoxicity. STING agonists enhanced NK and especially anti-mesothelin chimeric antigen receptor (CAR)-NK cell migration and killing, improving therapeutic activity. These studies reveal the fundamental importance of using human tumor samples to assess innate and cellular immune therapies. distinct consequences of STING agonist treatment in humans were uncovered by functionally profiling mesothelioma tumor explants with elevated tumor cell STING expression, supporting combinations with NK and CAR-NK cell therapies.

Advances in studying the human TIME using patient samples allow for development of immune therapies by treating patient derived organotypic tumor spheroids (PDOTS) and tissue fragment explants in short term culture (Jenkins et al., Cancer Discov. 8:196-215 (2018); Voabil et al., Nat. Med. 27:1250-1261 (2021)). To date, these platforms have focused on the anti-PD (L)-1 immune checkpoint and can parallel patient response, but they also offer promise in designing novel cancer immunotherapies. In contrast to patient derived xenografts grown in humanized mouse models, these systems provide the ability to interrogate the immune response within the native human tumor immune contexture and have the potential to study cell therapies without interference from the murine microenvironment. Furthermore, insights gained from modeling the direct human TIME may also help to close the translational gap for immune therapies that are effective in syngeneic mouse models but fail in clinical trials.

Multiple human cancer types have recently been shown to silence STING and the downstream interferon response to avoid immune detection (Ghosh et al., Cancer Cell 39:494-508 (2021); Kitajima et al., Cancer Discov. 9:34-45 (2019); Lau et al., Science 350:568-71 (2015)), clearly demonstrating an important role for tumor cell STING signaling in human cancer. Higher STING expression also correlates with better response to treatment across cancer types (Hayman et al., Nat. Commun. 12:2327 (2021); Zugazagoitia et al., Clin. Cancer Res. 26:4360-4368 (2020); Qi et al., Biosci. Rep. 40:BSR20202603 (2020); Chon et al., J. Cancer 10:4932-4938 (2019)). Yet how STING agonists impact tumor cells and different cell types in the human TIME has not been carefully examined and could inform development of novel therapeutic combinations, including cell therapy. This question is addressed herein by pursuing a large-scale study using PDOTS and developing methodology to conduct dynamic single cell RNA sequencing in tumor explants, as well as dissecting STING agonist response in an inflamed histotype.

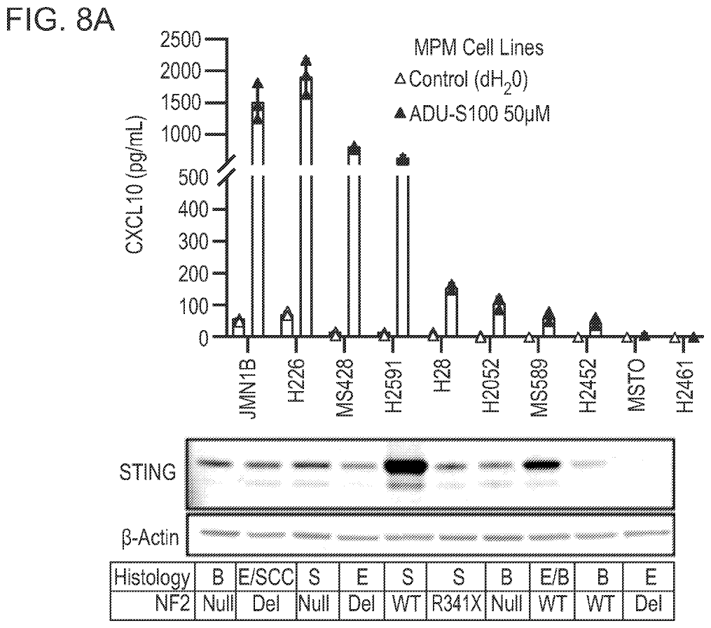

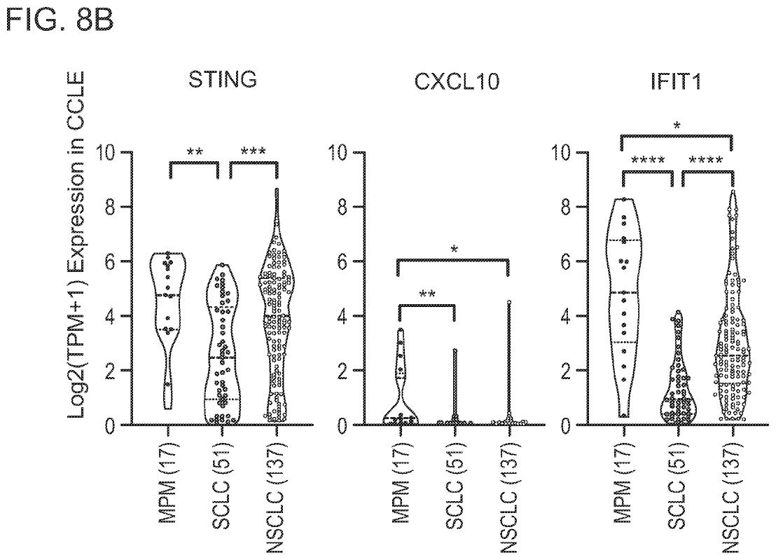

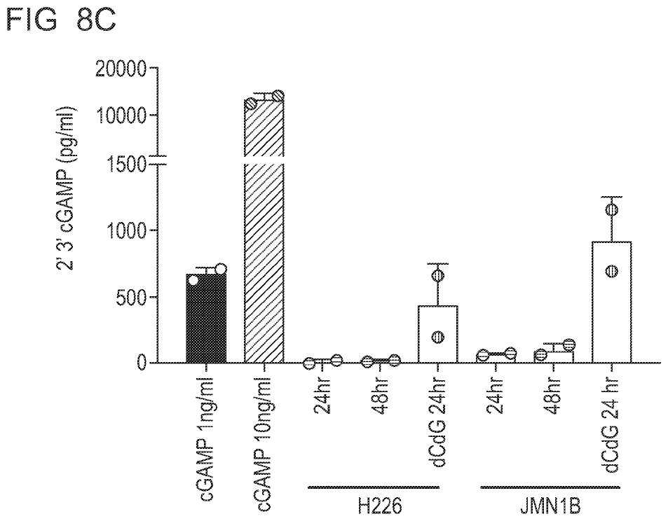

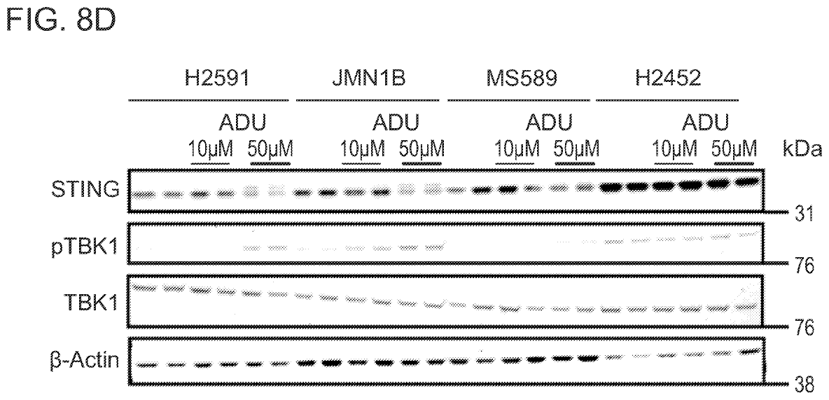

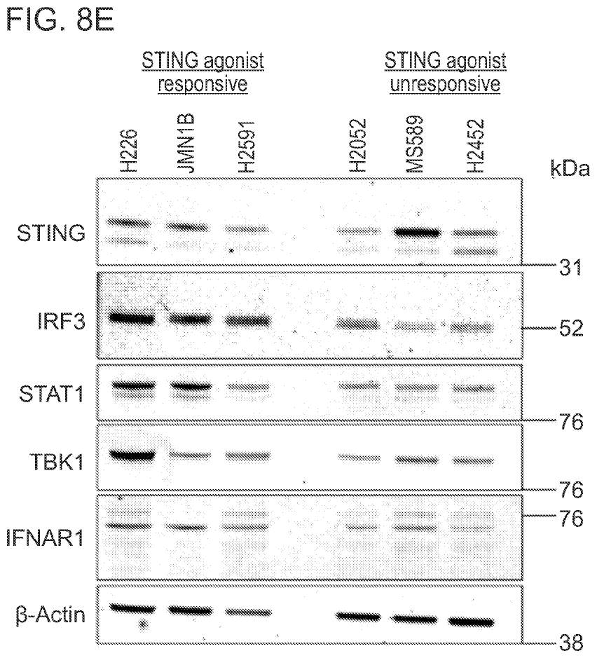

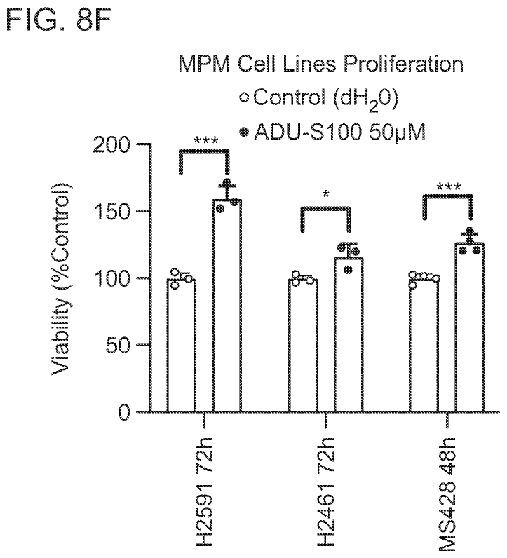

To identify human tumor histotypes with intact STING, immunohistochemistry profiling of 300 archival samples from diverse thoracic malignancies was performed (FIG. 1A). Among the malignancies evaluated, MPM expressed the highest levels of STING. MPM arises from the serosal lining of the lungs and carries a poor prognosis despite recent incorporation of combined checkpoint immunotherapy into standard care (Janes et al., N. Engl. J. Med. 385:1207-1218 (2021)). MPM demonstrated near-universal high expression of STING protein in tumor and stroma cells, in contrast to non-small cell lung carcinomas (NSCLC), thymomas, and especially small-cell lung carcinomas (SCLC) (Mahadevan et al., Cancer Discov. 11:1952-1969 (2021); Cañadas et al., Nat. Med. 24:1143-1150 (2018)) (FIG. 1A and FIG. 7A). STING was also highly expressed in benign pleura, consistent with its baseline upregulation in mesothelial cell types (FIG. 7B). MPM-derived cell lines expressed high levels of STING protein, but both cell lines and tumors failed to exhibit baseline cGAS-STING pathway activation as measured by37hosphoro-IRF3, CXCL10 and IFIT1 expression, and secreted 2′3′-cGAMP (FIG. 7C, FIG. 8A-FIG. 8D). Flow cytometry-based immune profiling of a large panel of resected MPM specimens further demonstrated robust immune infiltration in most tumors, but with features of exhaustion across multiple immune cell subsets including heterogeneous expression of the checkpoint proteins PD-1, TIM-3, and LAG-3 (FIG. 1B-FIG. 1D and FIG. 7D-FIG. 7E) (Awad et al., Cancer Immunol. Res. 4:1038-1048 (2016)). T-cell characterization revealed terminal differentiation consistent with exhaustion; monocyte/macrophage subtyping showed an abundance of intermediate cells; NK cell characterization showed diminished cytotoxic capacity (increased CD56 bright/CD16 low compared with circulating NK cells; FIG. 1D). Thus, MPM express high levels of STING and demonstrate an inflamed but exhausted TIME.

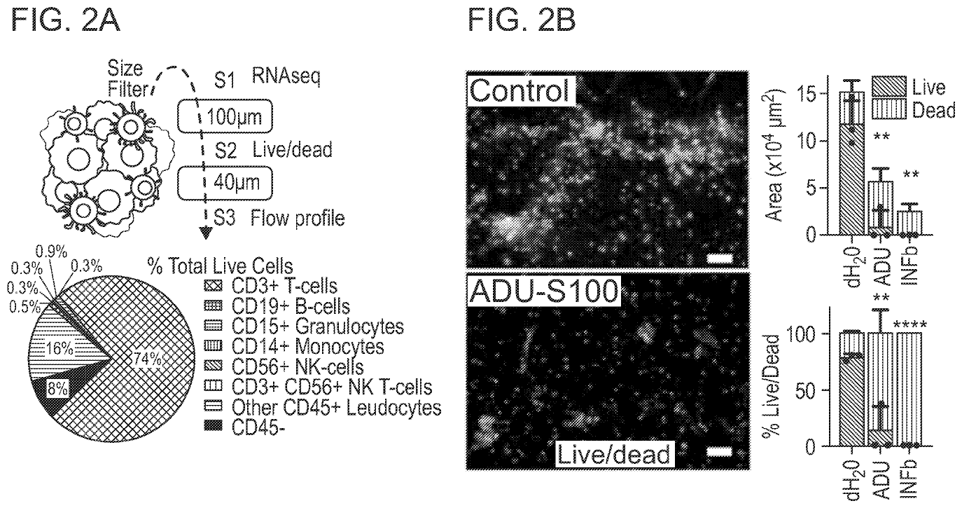

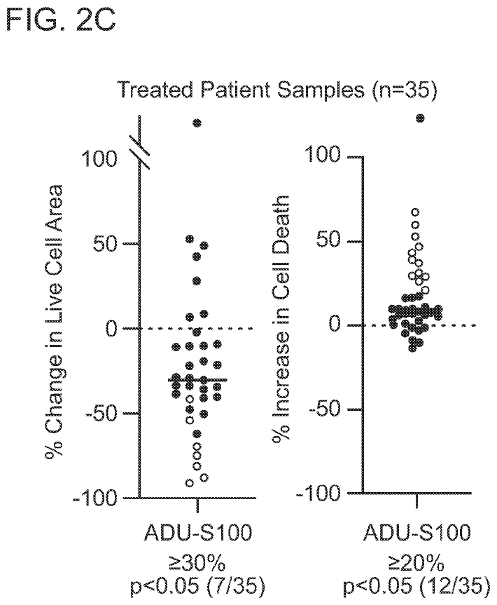

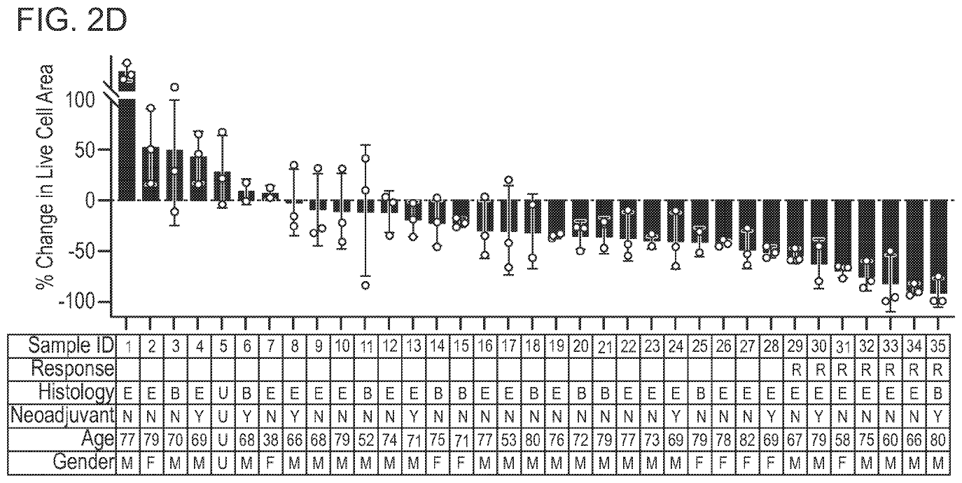

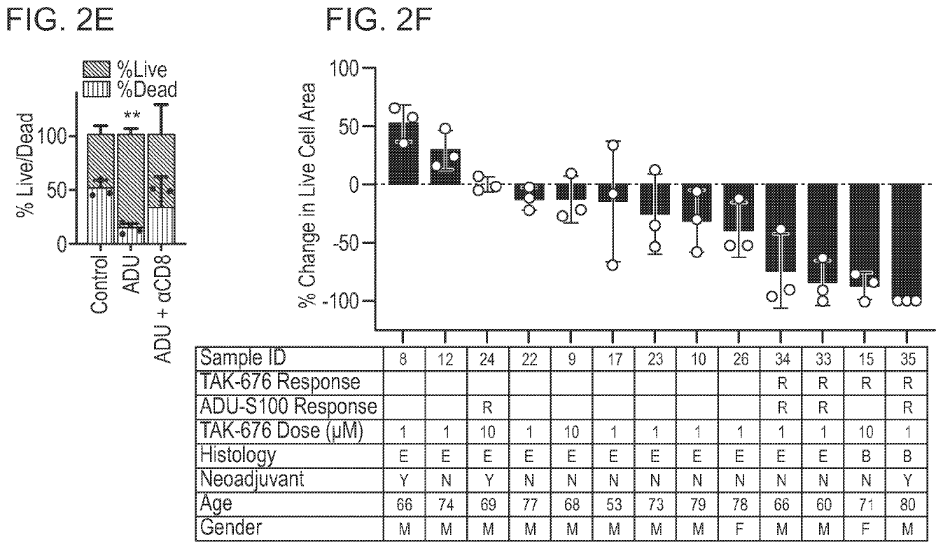

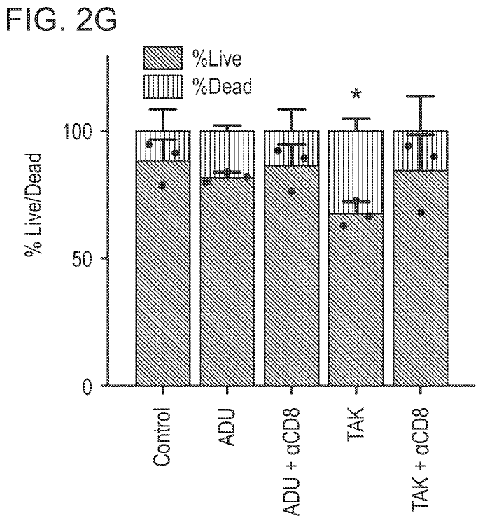

Despite lacking baseline STING pathway activation, multiple MPM cell lines treated with the clinical STING agonist ADU-S100 (Corrales et al., Cell Rep. 11:1018-30 (2015); Amouzegar et al., Cancers (Basel) 13:2695 (2021)) exhibited potent pathway activation, secreting high levels of CXCL10 (FIG. 8A-FIG. 8D). MPM cell lines with minimal response to clinical STING agonists in vitro exhibited reduced IRF3 transcription factor expression (FIG. 8A, FIG. 8D, and FIG. 8E). Overall, however, the high levels of inactive basal STING and pronounced induction of CXCL10 release by STING agonism suggests that STING signaling is primed to respond in MPM. STING agonism in human tumor specimens was next analyzed using freshly resected MPM tumor explant models that retain the associated TIME (FIG. 2A) (Jenkins et al., Cancer Discov. 8:196-215 (2018)). After processing, 40-100 μm (S2) PDOTS were suspended in collagen and treated for 6 days to assess response by live/dead immunofluorescence and cytokine production (Jenkins et al., Cancer Discov. 8:196-215 (2018)). ADU-S100 induced CXCL10 release and robust killing of PDOTS after 6 days of treatment in specific samples (FIG. 2A-FIG. 2B, and FIG. 9A-FIG. 9B). FIG. 2B shows cell area and percent live/dead quantification of each stain. T-test vs. dH20 control: **p<0.01, ****p<0.0001. Scale bars=100 μm. Importantly, STING activation in MPM cell lines cultured in vitro did not cause cytotoxicity, suggesting a contribution from the TIME (FIG. 8F). Overall, treatment of 35 patient specimens with ADU-S100 showed statistically significant cell death >20% above control (p<0.05) in 12/35 (34%) patient specimens, with 7 specimens approximating clinical criteria for response with >30% decrease in cell area, p<0.05 (FIG. 2C-FIG. 2D, and FIG. 9C). FIG. 2C shows responses by criteria for live cell area (≥30% decrease) and >20% increase in cell death, with p<0.05 by t-test between treated triplicate wells, ADU-S100 vs. dH20 control. FIG. 2D shows response by reduced live cell areal), epithelioid MPM (E), biphasic MPM (B), yes/no (Y/N) neoadjuvant treatment, and male/female (M/F). There was a non-significant trend toward higher response in patients who received neoadjuvant chemotherapy (HR 1.4, 95% CI 0.23-7.27, Chi Square p-0.72) and response did not correlate with histology (epithelioid and biphasic specimens were tested), age, or gender (Table 2). Furthermore, treatment with a second clinical-stage next generation systemic STING agonist, TAK-676, also showed promising activity ex vivo, with responses seen in 4/13 (31%) patient specimens (FIG. 2F). A potential correlation was observed between tumor CD8 abundance and treatment response to ADU-S100 ex vivo (R2-0.35, p<0.05; FIG. 10A), prompting the testing of the impact of CD8 neutralization. Indeed, anti-CD8 antibody treatment partially rescued ADU-S100 cytotoxicity in three patient specimens (FIG. 2E-FIG. 2G and FIG. 10A-FIG. 10B), motivating development of a higher resolution approach to understand the impact of STING agonism on different cells within the TIME.

TABLE 2
Patient demographics for ex vivo STING
agonist treatment of MPM tumors

| | Responder | | Non-responder | |
|---|---|---|---|---|
| Total Patients | 12 | | 23 | |
| Median Age (years) | 70.5 | | 73.5 | |
| Gender | | | | |
| Male | 8 | (66%) | 17 | (74%) |
| Female | 4 | (33%) | 5 | (22%) |
| Not recorded | 0 | | 1 | (4%) |
| Race | | | | |
| White | 12 | (100%) | 21 | (91%) |
| Black or African American | 0 | | 0 | |
| Other | 0 | | 1 | (4%) |
| Not recorded | 0 | | 1 | (4%) |
| Histology | | | | |
| Epithelioid | 9 | (75%) | 15 | (65%) |
| Biphasic | 3 | (25%) | 7 | (30%) |
| Sarcomatoid | 0 | | 0 | |
| Not recorded | 0 | | 1 | (4%) |
| Neoadjuvant Therapy | | | | |
| Yes | 4 | (33%) | 4 | (17%) |
| No | 8 | (66%) | 18 | (78%) |
| Not recorded | 0 | | 1 | (4%) |
| Adjuvant Therapy | | | | |
| Yes | 6 | (50%) | 11 | (48%) |
| No | 1 | (8%) | 7 | (30%) |
| Not recorded | 5 | (42%) | 5 | (22%) |
| AOPI | 7 | (58%) | 14 | (61%) |
| Hoechst/PI | 5 | (42%) | 9 | (39%) |

### Example 3: Dynamic scRNAseq of MPM Explants

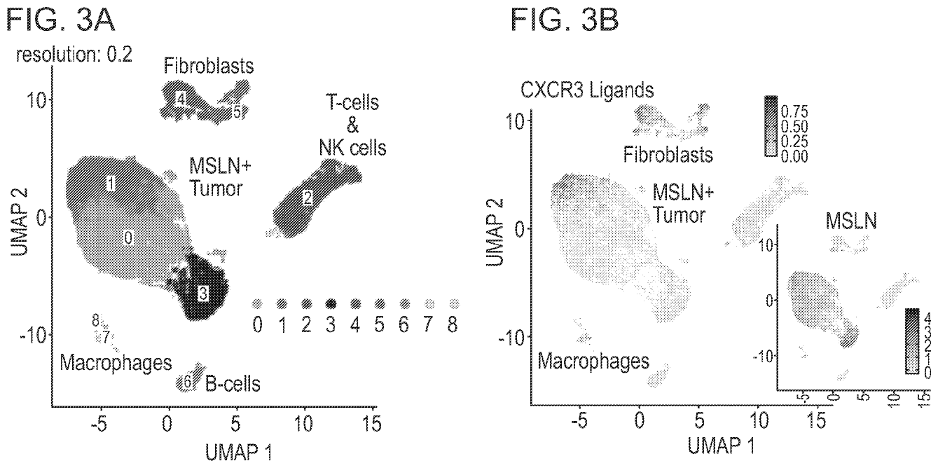

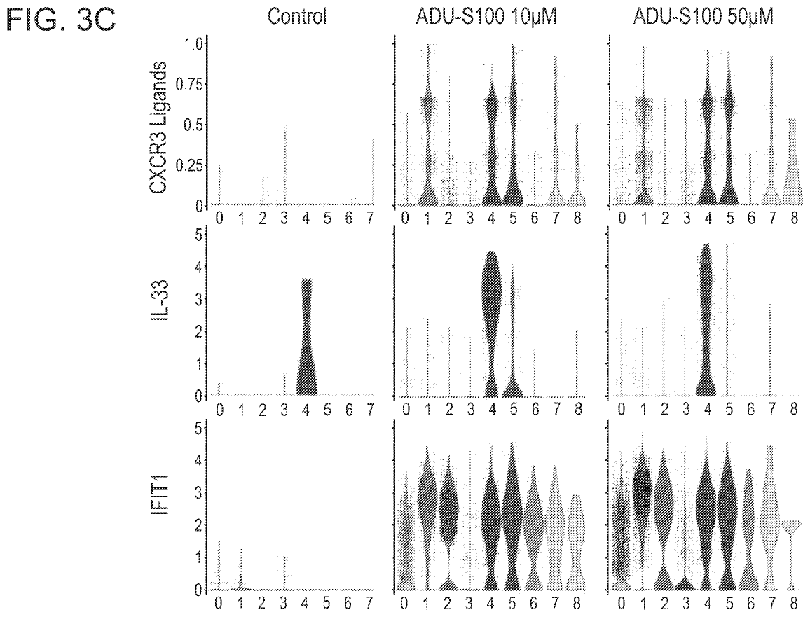

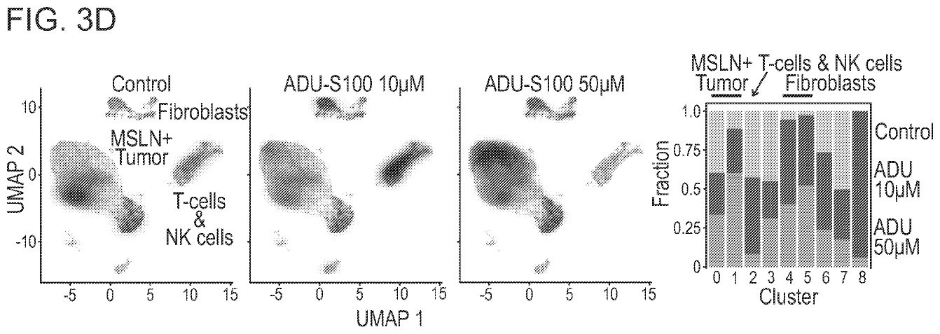

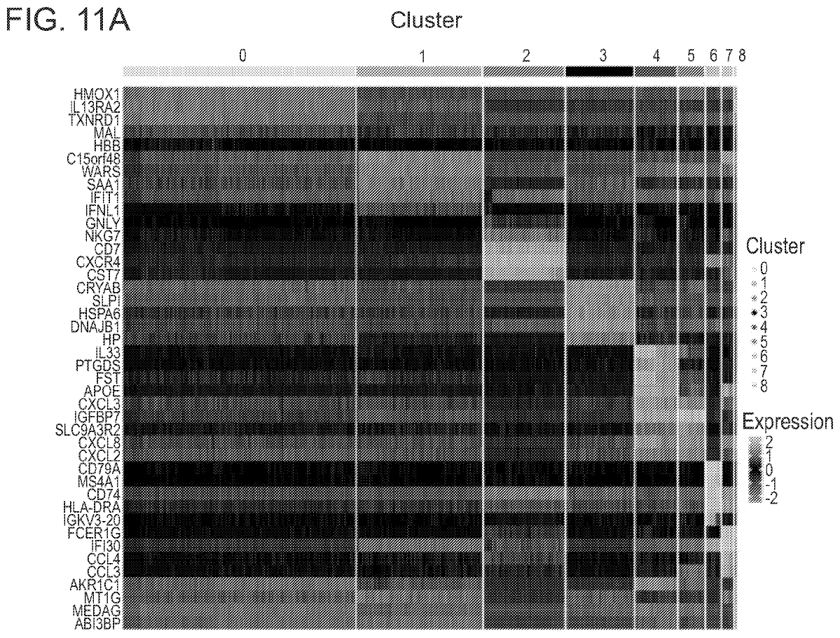

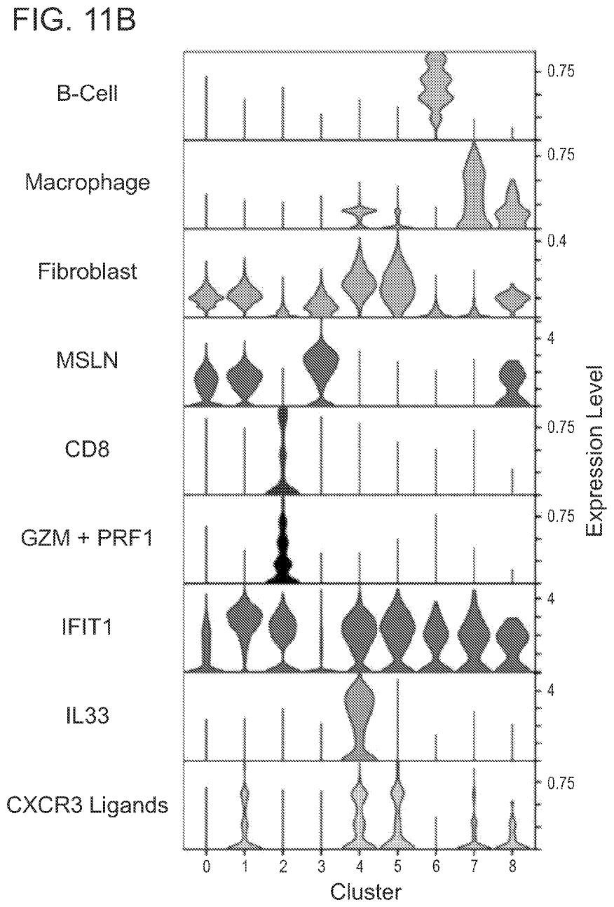

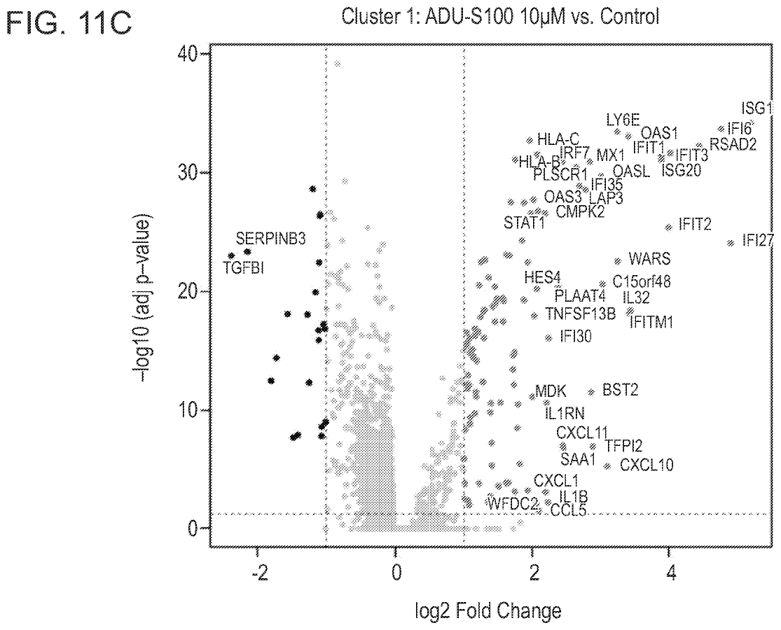

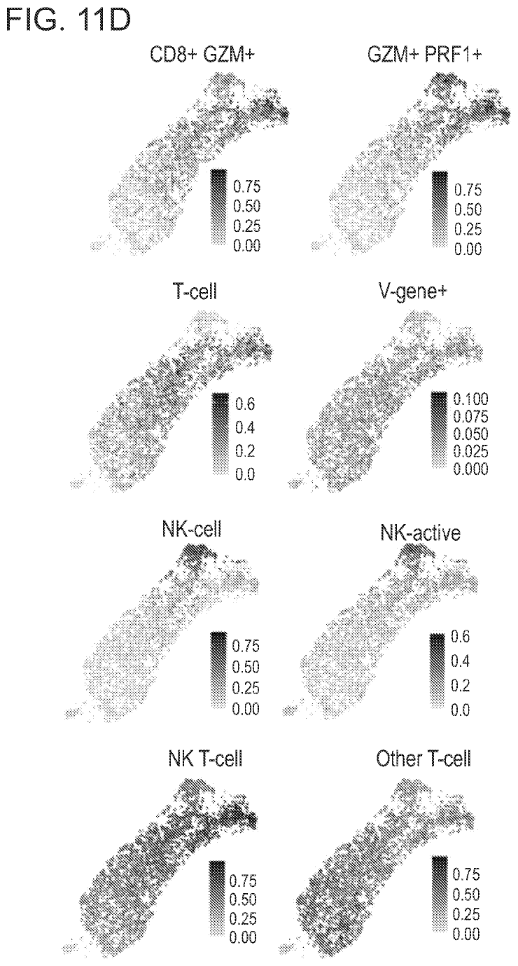

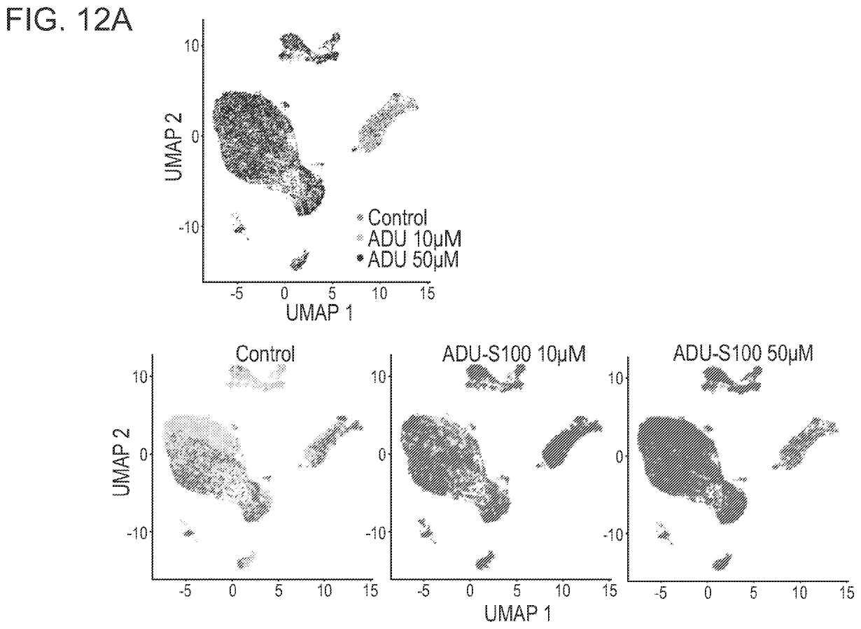

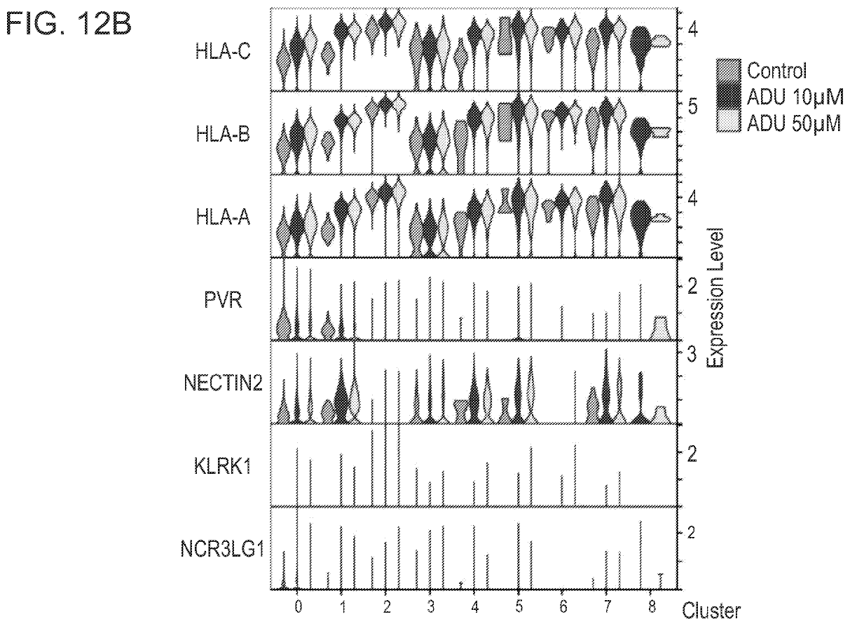

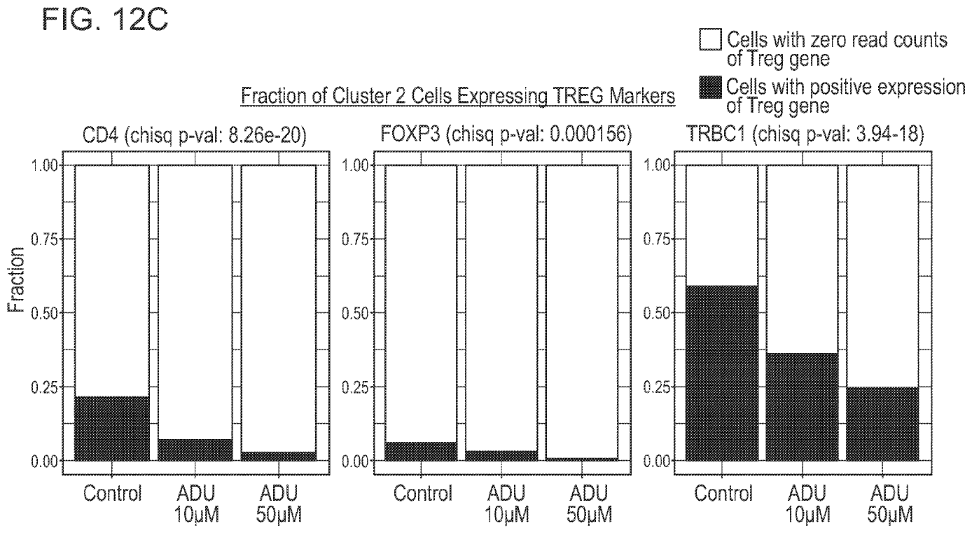

An adapted methodology to conduct dynamic single-cell RNA sequencing (scRNAseq) (Sehgal et al., J. Clin. Invest. 131:e135038 (2021)) was used, following 24-hours of STING agonist treatment, focusing on a specimen that exhibited modest, dose-dependent killing in response to ADU-S100, which was rescued by CD8 neutralization (FIG. 3A-FIG. 3D, FIG. 10B, FIG. 11A-FIG. 11D, and FIG. 12A-FIG. 12C). Fraction bar graph in FIG. 3D shows each cluster by treatment, normalized to number of cells per sample. Flow cytometry profiling prior to treatment demonstrated an average percentage of T-cells and an above average monocyte/macrophage population (FIG. 1B and FIG. 10C). IRF3 immunofluorescence also showed nuclear translocation following ADU-S100 treatment, confirming effective STING activation in PDOTS (FIG. 10E). Greater than 100 μm tumor fragments suspended in media were used for this short term scRNAseq analysis, confirming that size filtration did not change the leukocyte composition of each fraction (Jenkins et al., Cancer Discov. 8:196-215 (2018)) (FIG. 10D). UMAP clustering validated broad representation of tumor cell, fibroblast, and immune cell populations (FIG. 3A, FIG. 11A-FIG. 11B). Interestingly, ADU-S100 strongly induced CXCR3 ligand expression (CXCL9, CXCL10, CXCL11) primarily in a subset of mesothelin (MSLN) positive tumor cells (cluster 1) and cancer-associated fibroblasts (CAFs; clusters 4, 5), as compared with myeloid cell populations (clusters 7, 8; FIG. 3B-FIG. 3C). This analysis also revealed potent and unique STING agonist induction of IL-33 expression in CAFs, whereas other ISGs such as IFIT1 exhibited more widespread expression across cell populations, confirming broad target engagement (FIG. 3C). Differential expression analysis showed that the subset of MPM cells most highly expressing CXCR3 ligands in response to ADU-S100 (cluster 1) also displayed increases in numerous ISGs relative to other MPM cells (clusters 0, 3) and downregulated TGFB1 expression (FIG. 11C, Table 3-Table 4). Multiple granzyme and perforin positive T- and NK cells were identified within cluster 2 (FIG. 11D), consistent with the impact of CD8 neutralization and potential contribution of T- and/or NK cell mediated killing. However, analysis of abundance of cells originating from each individual sample (contour and fraction plots in FIG. 3D and FIG. 12A-FIG. 12C) demonstrated depletion of CD8 positive cell populations, including Tregs, following high-dose STING agonist treatment, contrasting with increased STING agonist response in MPM cluster 1. Analysis of NK cell ligands in tumor cells showed ADU-S100 dose-dependent increases in expression of HLA-A/B/C (inhibitory) coupled with increased NECTIN2 (CD112; activating) alongside decreased expression of the predominantly inhibitory ligand PVR (CD155) (Lupo et al., J. Hematol. Oncol. 13:76 (2020)) (FIG. 12B). These data reveal a prominent role for tumor cells and CAFs as targets of STING agonism, promoting release of T- and NK cell chemotactic factors and alteration of tumor/CAF cell state. Yet these results are also consistent with reports that excess STING activity can be toxic to T-cells (Cerboni et al., J. Exp. Med. 214:1769-1785 (2017); Larkin et al., J. Immunol. 199:397-402 (2017); Gulen et al., Nat Commun. 8:427 (2017)), suggesting that STING-mediated enhancement of tumor CXCR3 chemokine release may be countered by cytotoxicity in immune effector cells.

TABLE 3
40 Genes with Highest Increased Expression
Changes with STING Agonist Treatment

| 40 Most Increased Genes with Treatment (10 μM ADU-S100) | | | | | |
|---|---|---|---|---|---|
| Gene | p value | avg log2FC | pct.1 | pct.2 | p val adj |
| ISG15 | 3.23E−39 | 5.19 | 1.00 | 0.71 | 7.54E−35 |
| IFI27 | 4.01E−29 | 4.89 | 0.87 | 0.12 | 9.35E−25 |
| IFI6 | 1.01E−38 | 4.76 | 1.00 | 0.57 | 2.35E−34 |
| RSAD2 | 2.73E−37 | 4.43 | 0.97 | 0.02 | 6.37E−33 |
| IFIT3 | 1.04E−36 | 4.01 | 0.98 | 0.56 | 2.43E−32 |
| IFIT2 | 1.94E−30 | 3.99 | 0.92 | 0.39 | 4.53E−26 |
| IFIT1 | 1.83E−36 | 3.89 | 0.97 | 0.26 | 4.28E−32 |
| ISG20 | 4.04E−36 | 3.89 | 0.98 | 0.72 | 9.43E−32 |
| IL32 | 1.86E−23 | 3.43 | 0.82 | 0.23 | 4.34E−19 |
| IFITM1 | 3.04E−23 | 3.42 | 0.85 | 0.33 | 7.10E−19 |
| OAS1 | 4.07E−38 | 3.40 | 0.99 | 0.13 | 9.51E−34 |
| WARS | 1.33E−27 | 3.24 | 0.94 | 0.80 | 3.11E−23 |
| LY6E | 1.61E−38 | 3.24 | 1.00 | 0.95 | 3.77E−34 |
| CXCL10 | 2.50E−10 | 3.10 | 0.43 | 0.00 | 5.83E−06 |
| C15orf48 | 1.18E−25 | 3.02 | 0.91 | 0.54 | 2.76E−21 |
| OASL | 9.30E−35 | 3.01 | 0.94 | 0.05 | 2.17E−30 |
| TFPI2 | 5.10E−12 | 2.88 | 0.60 | 0.16 | 1.19E−07 |
| BST2 | 1.38E−16 | 2.86 | 0.61 | 0.02 | 3.22E−12 |
| MX1 | 5.77E−36 | 2.84 | 0.96 | 0.10 | 1.35E−31 |
| LAP3 | 1.26E−33 | 2.78 | 0.97 | 0.90 | 2.94E−29 |
| IFI35 | 6.12E−34 | 2.69 | 0.97 | 0.84 | 1.43E−29 |
| PLSCR1 | 1.58E−35 | 2.64 | 0.97 | 0.75 | 3.69E−31 |
| SAA1 | 7.21E−12 | 2.46 | 0.63 | 0.15 | 1.68E−07 |
| CXCL11 | 4.41E−12 | 2.45 | 0.48 | 0.00 | 1.03E−07 |
| IRF7 | 5.88E−36 | 2.44 | 0.98 | 0.82 | 1.37E−31 |
| PLAAT4 | 2.08E−25 | 2.38 | 0.90 | 0.49 | 4.86E−21 |
| IFI30 | 3.79E−21 | 2.24 | 0.75 | 0.15 | 8.85E−17 |
| IL1B | 2.62E−07 | 2.23 | 0.55 | 0.30 | 0.0061082 |
| IL1RN | 1.12E−15 | 2.21 | 0.74 | 0.28 | 2.62E−11 |
| CXCL1 | 3.53E−08 | 2.20 | 0.61 | 0.30 | 0.0008241 |
| CMPK2 | 1.25E−31 | 2.19 | 0.89 | 0.03 | 2.91E−27 |
| CCL5 | 1.26E−06 | 2.10 | 0.29 | 0.00 | 0.0294404 |
| HLA-B | 1.84E−36 | 2.09 | 1.00 | 1.00 | 4.29E−32 |
| STAT1 | 8.11E−32 | 2.09 | 0.94 | 0.57 | 1.89E−27 |
| HLA-C | 1.34E−36 | 2.08 | 1.00 | 1.00 | 3.12E−32 |
| HES4 | 2.74E−25 | 2.07 | 0.91 | 0.56 | 6.40E−21 |
| WFDC2 | 3.52E−07 | 2.06 | 0.73 | 0.62 | 0.0082103 |
| TNFSF13B | 5.30E−23 | 2.03 | 0.77 | 0.08 | 1.24E−18 |
| OAS3 | 9.16E−33 | 2.02 | 0.93 | 0.28 | 2.14E−28 |
| MDK | 3.49E−16 | 2.01 | 0.72 | 0.28 | 8.15E−12 |

TABLE 4
20 Genes with Most Decreased Expression
Changes with STING Agonist Treatment

| 20 Most Decreased Genes with Treatment | | | | | |
|---|---|---|---|---|---|
| Gene | p value | avg log2FC | pct.1 | pct.2 | p val adj |
| TGFBI | 4.65E−28 | −2.38 | 0.57 | 0.98 | 1.09E−23 |
| SERPINB3 | 2.15E−28 | −2.15 | 0.52 | 0.98 | 5.02E−24 |
| KRT19 | 1.57E−17 | −1.81 | 0.83 | 1.00 | 3.66E−13 |
| SERPINB7 | 1.88E−19 | −1.73 | 0.58 | 0.95 | 4.40E−15 |
| CCDC80 | 3.66E−23 | −1.57 | 0.79 | 1.00 | 8.56E−19 |
| ANGPTL4 | 9.13E−13 | −1.48 | 0.33 | 0.72 | 2.13E−08 |
| IGFBP4 | 5.58E−13 | −1.42 | 0.75 | 0.97 | 1.30E−08 |
| GPI | 4.12E−23 | −1.27 | 0.71 | 0.98 | 9.62E−19 |
| SERPINB4 | 2.22E−17 | −1.25 | 0.46 | 0.90 | 5.19E−13 |
| CEMIP | 1.11E−33 | −1.20 | 0.14 | 0.71 | 2.59E−29 |
| LDHA | 5.51E−25 | −1.16 | 0.98 | 1.00 | 1.29E−20 |
| PABPC1 | 8.78E−22 | −1.11 | 0.95 | 1.00 | 2.05E−17 |
| TPI1 | 5.70E−21 | −1.11 | 0.96 | 1.00 | 1.33E−16 |
| RPSA | 1.71E−27 | −1.10 | 0.97 | 1.00 | 4.00E−23 |
| RPS29 | 1.97E−31 | −1.10 | 0.89 | 1.00 | 4.61E−27 |
| RPL41 | 1.57E−31 | −1.09 | 0.95 | 1.00 | 3.66E−27 |
| NDRG1 | 6.92E−13 | −1.07 | 0.48 | 0.90 | 1.62E−08 |
| ID1 | 1.11E−13 | −1.07 | 0.29 | 0.72 | 2.60E−09 |
| PGK1 | 2.74E−22 | −1.04 | 0.92 | 1.00 | 6.39E−18 |
| TUBA1A | 6.65E−22 | −1.02 | 0.23 | 0.75 | 1.55E−17 |

### Example 4: STING Agonists are Toxic to Human T-Cells

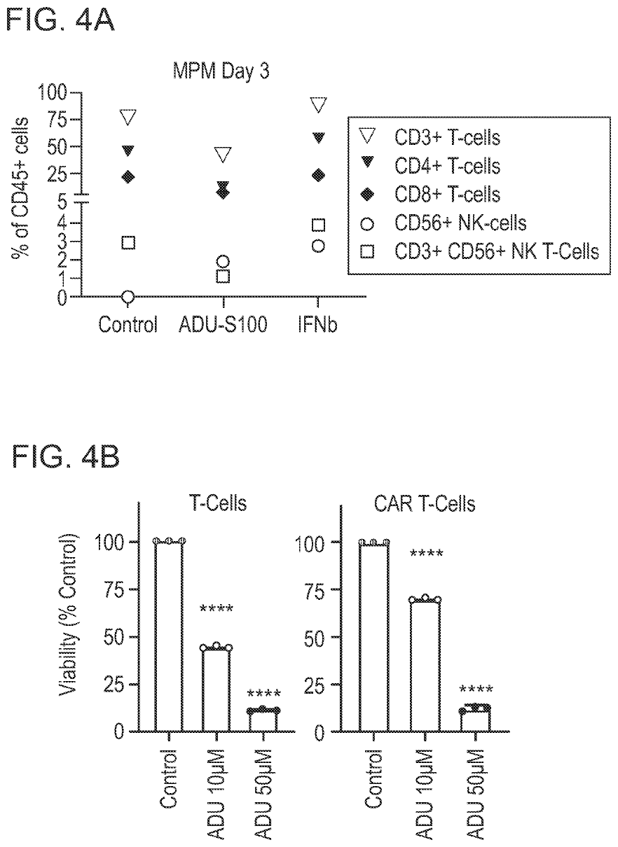

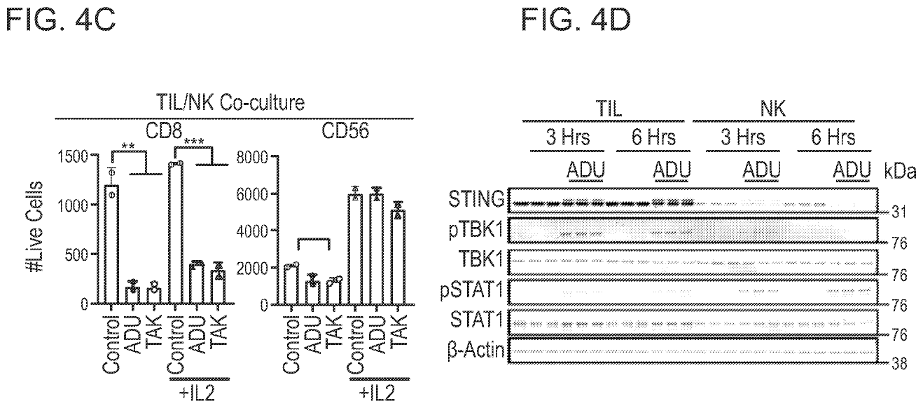

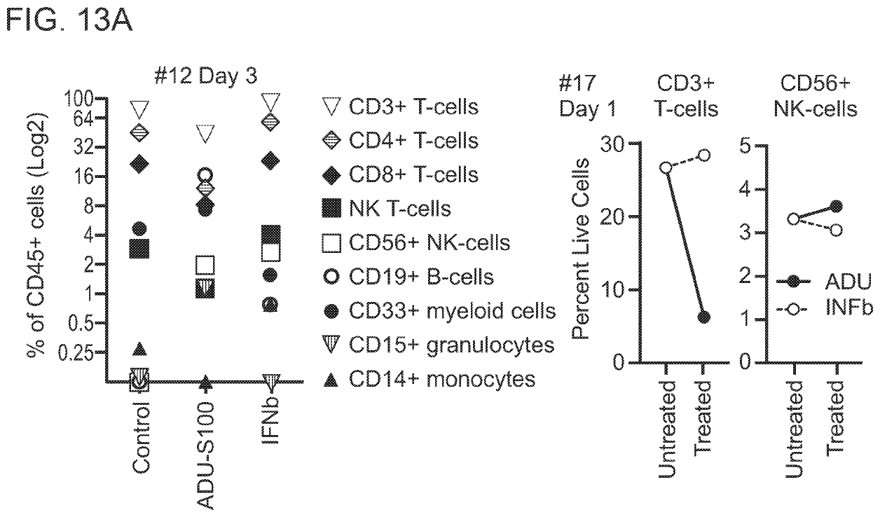

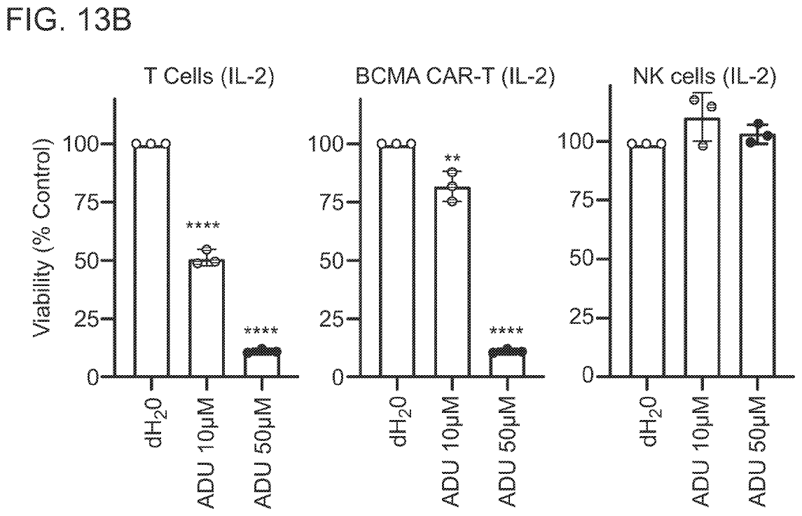

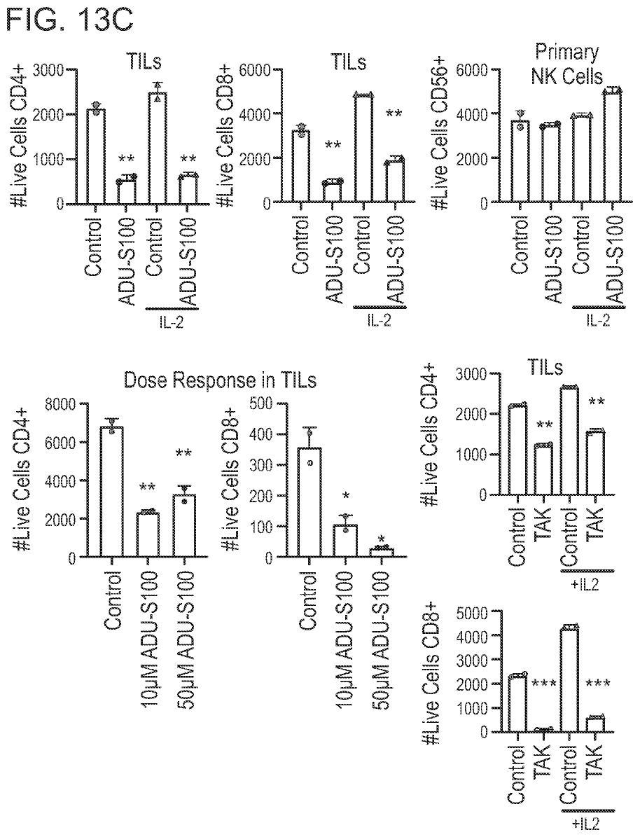

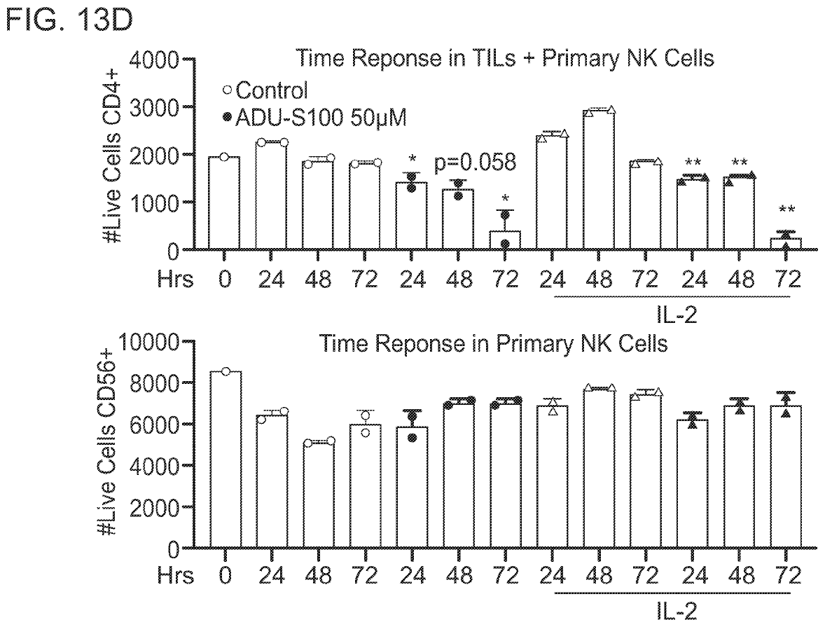

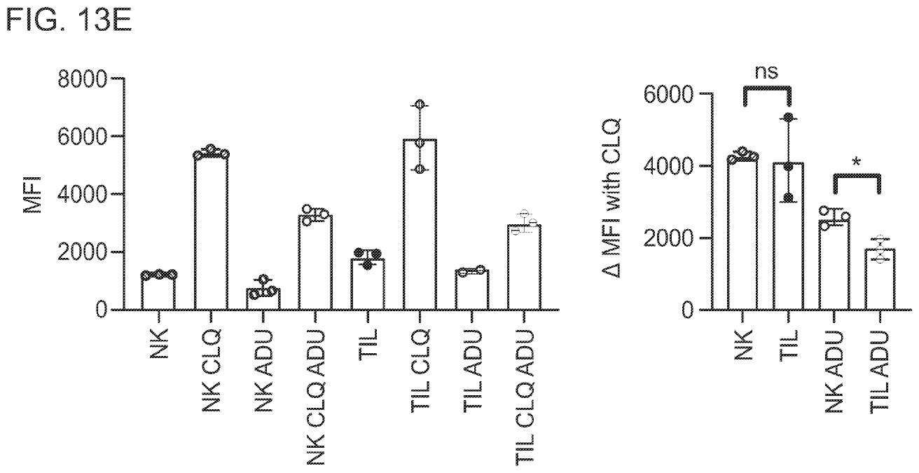

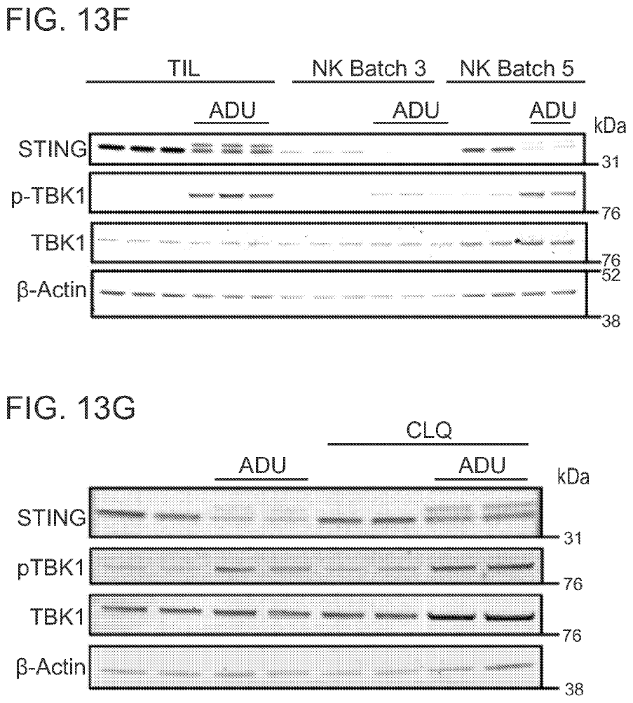

To explore this observation further, STING induced cytotoxicity in T-cells was evaluated (Cerboni et al., J. Exp. Med. 214:1769-1785 (2017); Larkin et al., J. Immunol. 199:397-402 (2017); Gulen et al., Nat. Commun. 8:427 (2017)) using the models described herein, as well as cytotoxicity in other immune cell types. ADU-S100 treatment, in contrast to downstream IFNβ exposure, was cytotoxic to T-cells as measured by flow cytometry in MPM tumor explants, which increased over time from 24 to 72 hours of STING agonist treatment (FIG. 4A and FIG. 13A). T-cells purified from peripheral blood with or without expression of a B-cell maturation antigen chimeric antigen receptor (BCMA CAR) also demonstrated dose-dependent cytotoxicity after STING agonist treatment (FIG. 4B, FIG. 13B), likely limiting combinations of STING agonists with CAR T-cells as recently proposed (Xu et al., J. Exp. Med. 218:e20200844 (2021); Smith et al., J. Clin. Invest. 127:2176-2191 (2017)).

Although NK T-cells were also sensitive to STING agonism (FIG. 4A), human NK cells showed no significant cytotoxicity from STING agonist treatment, regardless of culture in IL-2 or co-culture with TILs (FIG. 4A-FIG. 4C and FIG. 13A-FIG. 13D). FIG. 4C shows flow cytometry after 72-hour treatment with 50 μM ADU-S100, 10 μM TAK-676 or dH20 control+/−200 U/mL IL-2, with gating for live cells out of 10,000 total events expressing CD8 or CD56. Batch 3 primary NK cells expanded from PBMCs and TILs from a 66-year-old man with stage I NSCLC. One-way ANOVA p<0.01 with corrected pairwise comparisons: **p<0.01, ***p<0.001. These findings held across human tumors and expanded primary NK cells, even after 72 hours of high dose ADU-S100 or TAK-676 exposure. NK cells principally rely on metabolism via oxidative phosphorylation (Keppel et al., J. Immunol. 194:1954-62 (2015)) requiring ongoing autophagic flux (Wang et al., Nat. Commun. 7:11023 (2016)), whereas T-cells depend on glycolysis and tolerate defective autophagy (Clarke et al., Nat. Rev. Immunol. 19:170-183 (2019)). Indeed, consistent with their elevated autophagic flux following ADU-S100 treatment (FIG. 13E), levels of STING protein were lower in NK cells and STING was rapidly degraded within 3-6 hours of STING agonism (FIG. 4D and FIG. 13F-FIG. 13G). In contrast, T-cell STING was phosphorylated and activated by ADU-S100, but minimally degraded (FIG. 4D and FIG. 13F). Autophagy-dependent STING recycling in NK cells was confirmed, since treatment with chloroquine (CLQ), which blocks autophagic flux by inhibiting autophagosome fusion with the lysosome (Mauthe et al., Autophagy 14:1435-1455 (2018)), prevented ADU-S100 induced STING degradation (FIG. 13G). Thus, whereas STING agonist treatment causes effector T-cell cytotoxicity, primary NK cells remain largely unscathed.

### Example 5: STING Agonists Enhance NK Cell Therapies

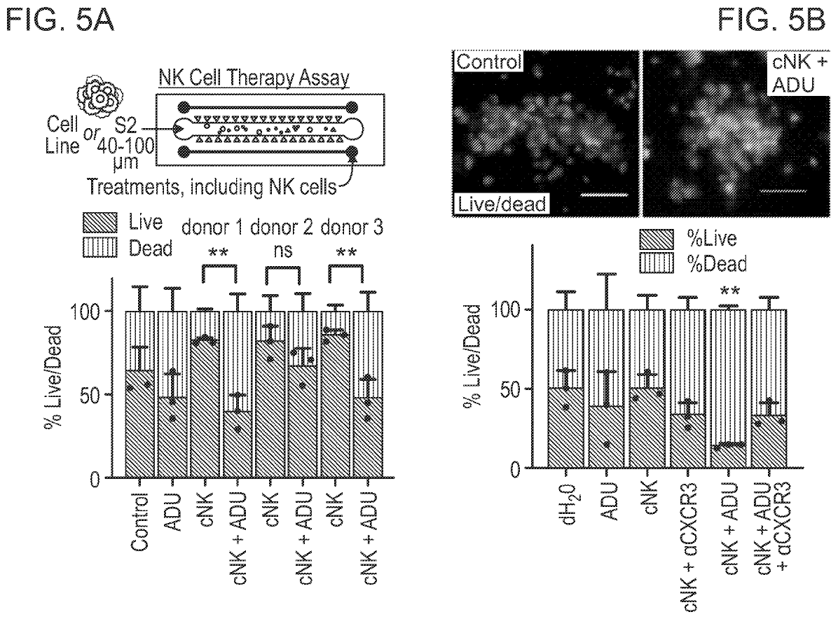

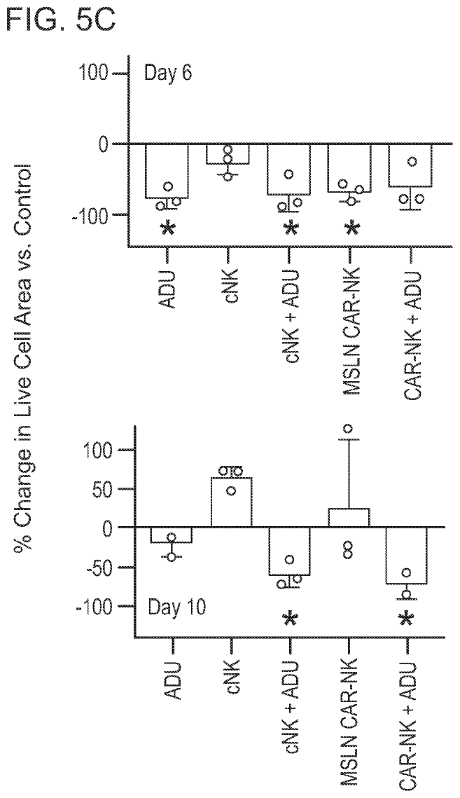

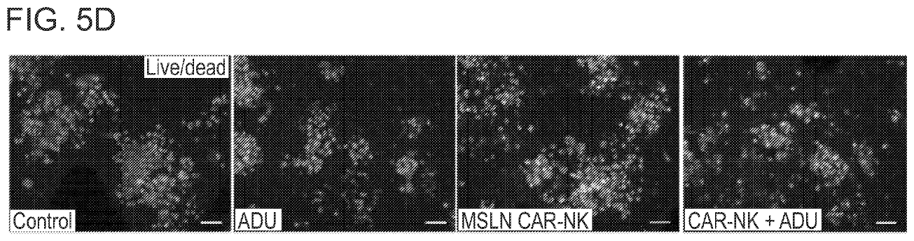

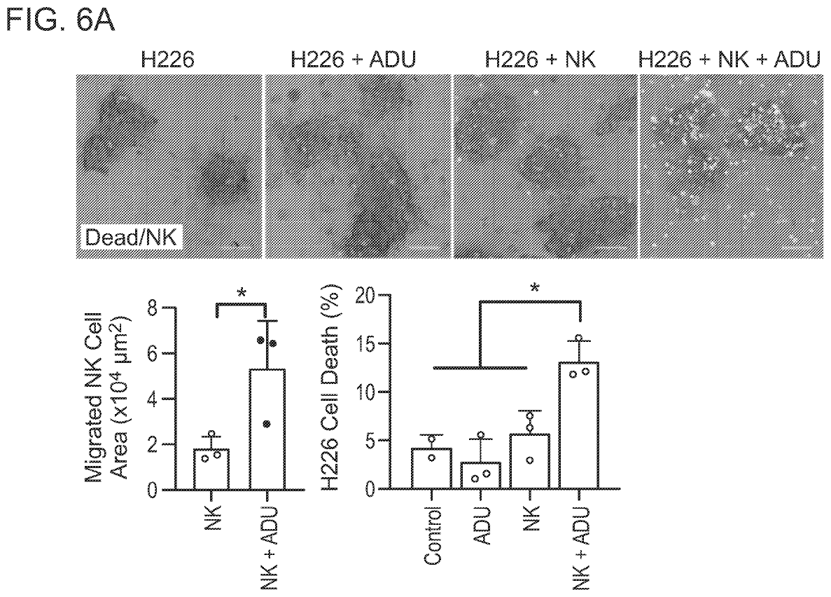

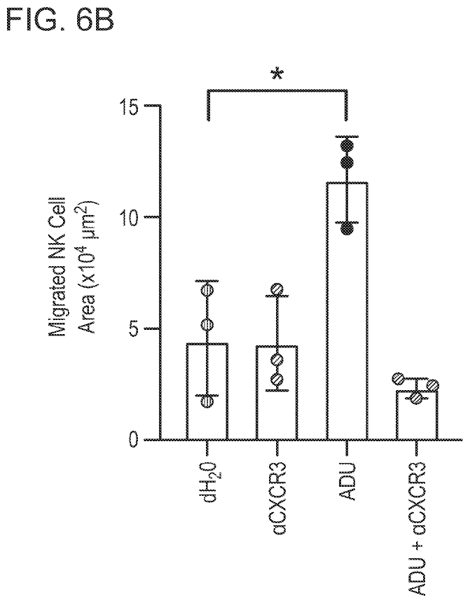

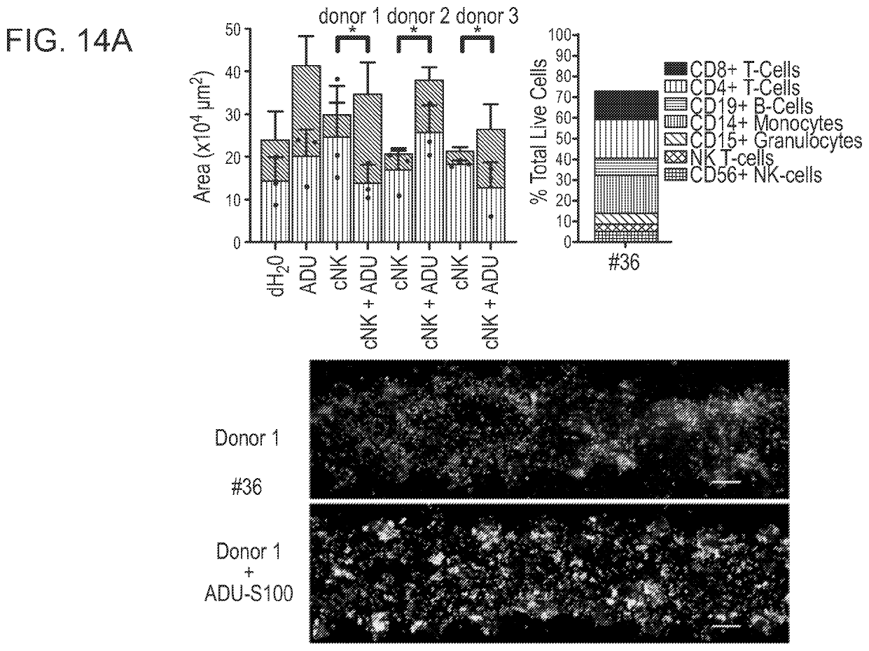

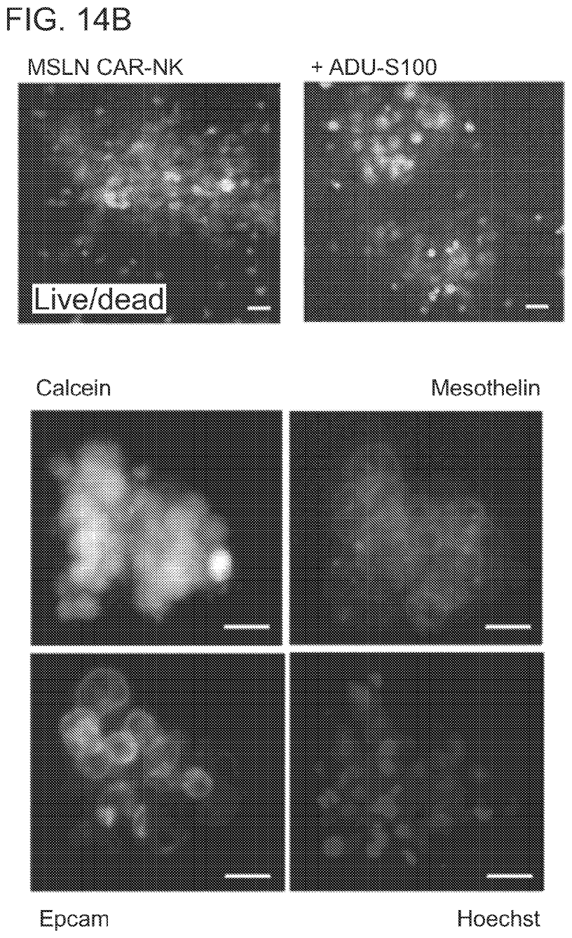

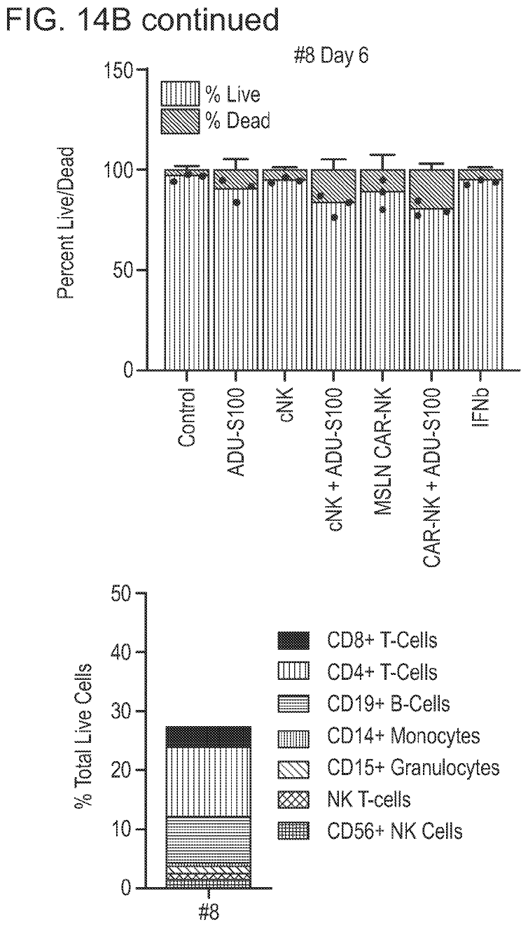

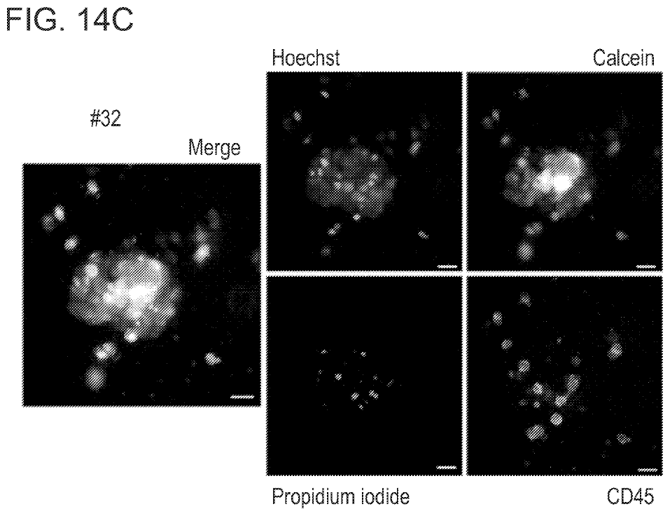

NK cells are generally low in number in MPM specimens (FIG. 1B), and also potentially restrained by inhibitory signals on tumor cells such as MHC-I, which may increase following STING agonist treatment (FIG. 12B). STING agonism combined with adoptive transfer of primary or engineered NK cells was next examined to determine if this represents a promising therapeutic strategy by coupling tumor CXCR3 chemokine release with an effector cell type resistant to STING agonist cytotoxicity. Whereas addition of primary NK cells alone to the treatment channel of microfluidic devices failed to kill MPM PDOTS, combined treatment with ADU-S100 significantly enhanced primary NK cell response using cells from 2 out of 3 donors (FIG. 5A and FIG. 14A). Moreover, ADU-S100 mediated enhancement of NK cytotoxicity in MPM PDOTS was impaired by CXCR3 neutralization (FIG. 5B). To further overcome a potentially inhibitory role of MHC-I, the next focus was on anti-MSLN CAR strategies being developed clinically for MPM (Janes et al., N. Engl. J. Med. 385:1207-1218 (2021)), utilizing NK cells instead of T-cells as an alternative vector for anti-MSLN CAR (FIG. 16A). Indeed, using a MSLN brightly positive MPM specimen, it was discovered that anti-MSLN CAR NK cells significantly augmented ADU-S100 activity at 6 days in a PDOTS sample that was minimally responsive to ADU-S100 treatment alone (FIG. 14B). Furthermore, combined addition of NK and especially CAR-NK cell therapy with ADU-S100 promoted deep growth suppression of MLSN+ PDOTS over time in culture, contrasting with day 10 rebound that occurred following single agent therapy (FIG. 5C; FIG. 14C). These data confirm that continuous STING agonist exposure is not toxic to NK cell therapies and suggest that it may enhance activity, especially in combination with anti-MSLN CAR NK cells.

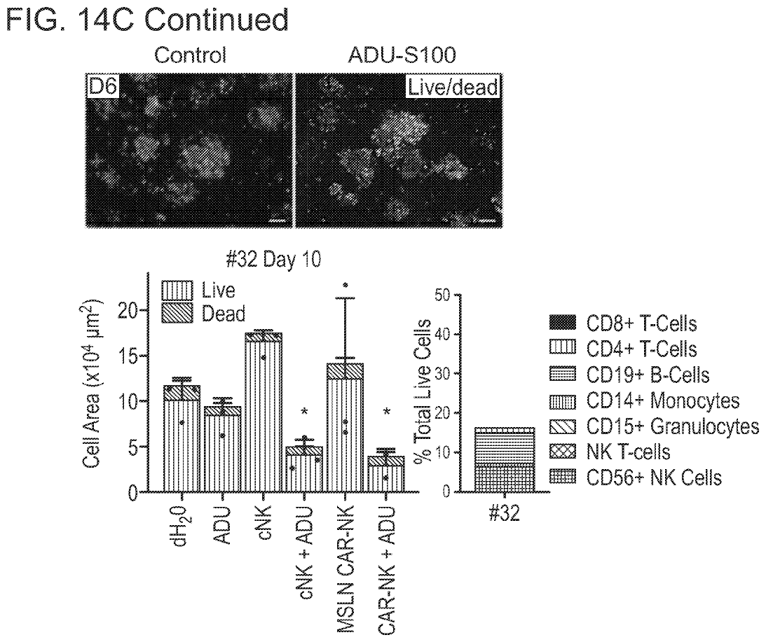

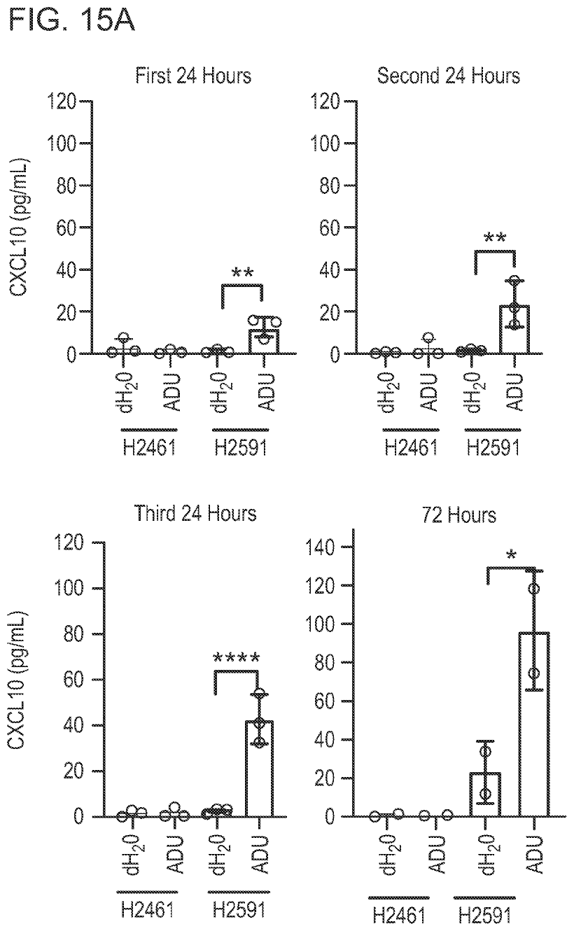

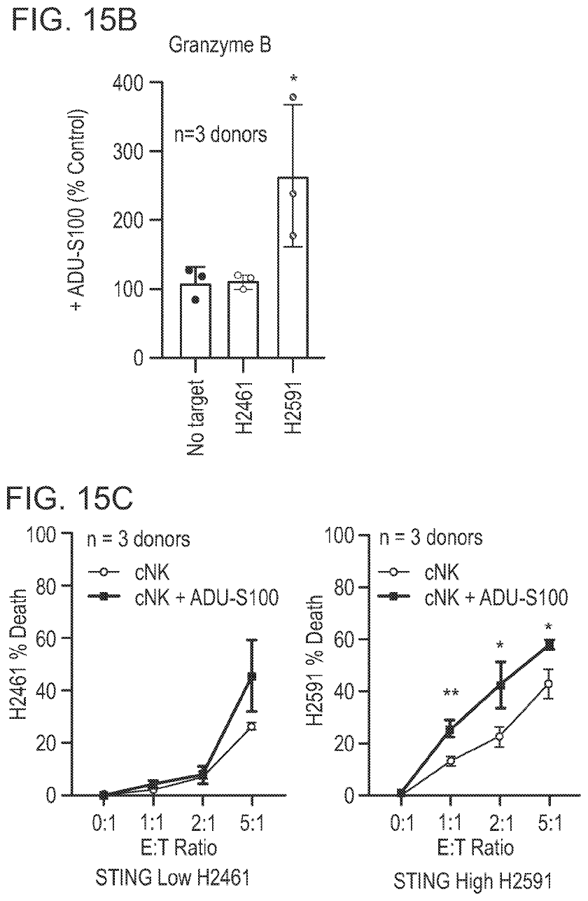

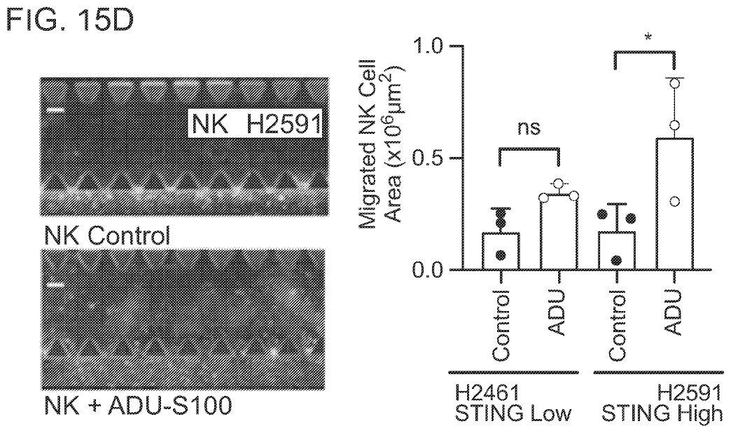

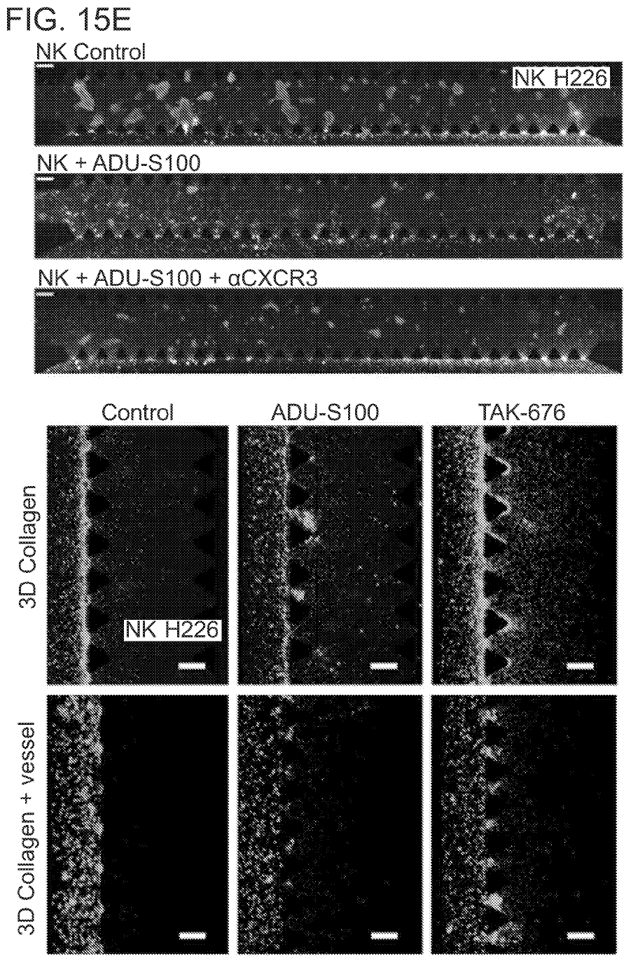

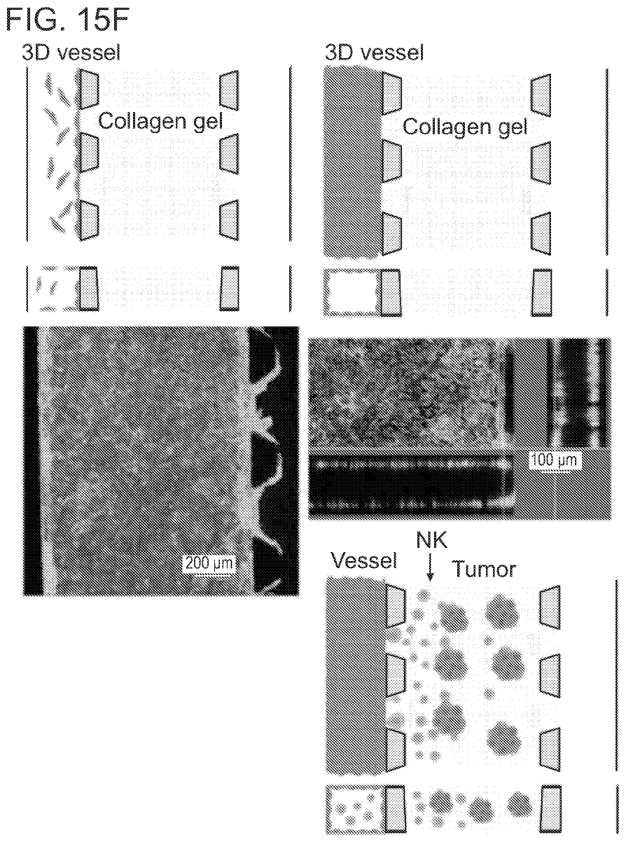

To isolate the role of tumor cells and further validate these findings, MPM cell lines that highly express STING and secrete CXCL10 over time were used during STING agonist treatment (H2591, H226, MS428) or uniquely lack STING expression and do not respond to STING agonism (H2461; FIG. 8A, FIG. 15A) and compared NK cell migration and killing−/+ADU-S100 treatment in vitro (FIG. 6, FIG. 15A-FIG. 15F, FIG. 16A-FIG. 16C). STING agonism enhanced granzyme release by NK cells (FIG. 15B) and apoptosis of tumor cells (FIG. 6A-FIG. 6D, FIG. 15C, FIG. 16B-FIG. 16C) only in co-culture with MPM cells expressing STING. These findings were consistent across E:T ratios (FIG. 15C and FIG. 16B), 2D and 3D culture (FIG. 6A-FIG. 6D), and varied somewhat with NK cell donors like the experiments using patient specimens (FIG. 5A-FIG. 5D, FIG. 6A-FIG. 6D, FIG. 14A-FIG. 14C, FIG. 15C, FIG. 16C). STING-agonist induced increases in NK cell migration towards tumor cells were rescued by treatment with a CXCR3 neutralizing antibody (FIG. 6B, FIG. 15E), similar to the results seen in NK cytotoxicity in MPM patient specimens (FIG. 5B). To model NK cell migration across a vascular barrier, human umbilical vein endothelial cells (HUVEC) were cultured in 3D to form a vessel before assessing physiologic NK cell migration out of the vessel and through collagen to reach MPM tumor cell lines (FIG. 15F). ADU-S100, and especially TAK-676, enhanced NK cell migration in the presence and absence of the vascular barrier, with expected decreases in total migration through the vessel (FIG. 6C).

Finally, since the PDOTS data suggested that mesothelin CAR construct expression could enhance adoptive NK cell therapy in MPM when combined with STING agonists (FIG. 5C), this treatment combination was evaluated in vitro to assess cytotoxicity. Anti-mesothelin CAR expression further enhanced in vitro NK cell killing and combined with ADU-S100 treatment to cause the most tumor cell death (FIG. 6D and FIG. 16A-FIG. 16C). Taken together, these data reveal that STING agonism in STING positive human tumor models activates anti-viral signaling programs, promotes CXCR3 ligand chemokine release from tumor cells, enhances NK cell recruitment and cytotoxicity, and may have potent combinatorial activity with NK cell therapies.

Evaluating human tumors in short-term cultures that preserve the tumor-immune microenvironment can overcome some of the limitations of mouse models, patient-derived xenografts, and passaged organoids to potentially inform clinical trials of next-generation immunotherapy combinations including cell therapies. Described herein are dynamic single-cell RNA sequencing of ADU-S100-treated human tumor explants to dissect the mechanism of action of a clinical stage STING agonist. STING agonism engages its target in most cells of the TIME, but principally drives CXCR3 chemokine activation in tumor cells and cancer-associated fibroblasts, while causing T-cell cytotoxicity. Blunting of effector T-cell activity is an unexpected consequence that could contribute to the disappointing clinical activity of STING agonists to date in humans. However, these studies reveal that this drawback can be overcome with the addition of NK cell therapies (Myers et al., Nat. Rev. Clin. Oncol. 18:85-100 (2021)), which benefit from STING agonist enhancement of NK cell migration and killing.

More generally, available data from mouse models and clinical trials of injectable STING agonists support a complex interplay of STING activation in the TIME. Indeed, cell types other than CD8 T-cells such as monocytes and NK cells could be involved in the infrequent clinical responses to STING agonists reported in patients (Harrington et al., Annals of Oncology 29:viii712 (2018); Meric-Bernstam et al., Journal of Clinical Oncology 37:2507 (2019)). Moreover, recent work in syngeneic mouse models has uncovered an important role for NK cells in tumor control mediated by the endogenous STING agonist ligand 2′3′-cGAMP (Marcus et al., Immunity 49:754-763 (2018); Nicolai et al., Sci. Immunol. 5:eaaz2738 (2020)). These data implicate NK cells in murine STING agonist response in vivo, which is otherwise difficult to model using artificial humanized mouse xenografts. In a related manuscript describing the pre-clinical activity of TAK-676, we observe enhanced trafficking and activation of NK cells following systemic TAK-676 administration in murine models. TAK-676 treatment was also especially potent at overcoming a human vascular barrier in our ex vivo model.

Adding to the complexity of injectable STING agonist trials is a potential threshold effect for cytokine release whereby tumor cell STING activation crosses from metastasis promoting (Chen et al., Nature 533:493-498 (2016); Bakhoum et al., Nature 553:467-472 (2018)) to immune rejection. In MPM, minimal baseline phosphorylation of downstream IRF3 was observed in patient specimens (FIG. 7C) and negligible extracellular 2′3′-cGAMP released from cell lines (FIG. 8C) suggesting potentially low contribution from basal cGAS-STING signaling to the observed immune exhaustion (FIG. 1A-FIG. 1D). But these data suggest instead that elevated tumor cell STING expression in MPM creates a particular vulnerability to therapeutic STING agonism, especially when coupled with NK cell therapy. This vulnerability could also extend to other tumor types with high basal STING expression, or to STING silenced tumors treated with epigenetic inhibitors (Falahat et al., Proc. Natl. Acad. Sci. U.S.A. 118:e2013598118 (2021)).

Clinical development of STING agonists is further limited by the narrow therapeutic window for injectable agents, which are rapidly cleared (Harrington et al., Annals of Oncology 29:viii712 (2018); Meric-Bernstam et al., Journal of Clinical Oncology 37:2507 (2019)). While novel slow-release and systemic formulations of STING agonists could solve some of these issues (Amouzegar et al., Cancers (Basel) 13:2695 (2021)), the data disclosed herein indicate that constant exposure is likely to kill endogenous effector T-cells, and also limit combinations with adoptively transferred transgenic TCR-T or CAR T-cell therapies (Xu et al., J. Exp. Med. 218:e20200844 (2021); Smith et al., J. Clin. Invest. 127:2176-2191 (2017)). Instead, the findings described herein, that NK cells are resistant to constant high-dose STING agonist exposure, and in fact activated and recruited to kill MPM cells, support this novel immunobiology and provide a straightforward combinatorial approach with NK cell therapies to develop clinically. Furthermore, the benefits of adding a STING agonist to NK cell therapies may not necessarily depend on the CAR construct, allowing for combinations with a variety of emerging NK effector cells (Myers et al., Nat. Rev. Clin. Oncol. 18:85-100 (2021)). Treatments to enhance native NK cell activation could also be effective in combination with STING agonists. Interestingly, while the single-cell RNA sequencing data show NK inhibitory MHC I upregulation, they also reveal specific modulation of CD112 and CD155 that could converge to activate NK cells (FIG. 12B), especially when coupled with adoptive NK cell therapies (FIG. 5A-FIG. 5D and FIG. 6A-FIG. 6D).

Timing and sequencing of combination immune therapies remain critical, as burst-dose STING agonism (alongside NK cell infusion) could prevent T-cell cytotoxicity and allow for later cross-priming of T-cells via NK to dendritic cell to T-cell crosstalk that enhances antitumor immunity. The potent/specific TBK1 inhibitor described in Jenkins et al., Cancer Discov. 8:196-215 (2018) activates T cells and may be a combinatorial therapy with the inventive therapies described herein.

### Example 6: CXCR3 Overexpression in CAR-NK Cells Primes Migration and Homing into the Tumor Microenvironment

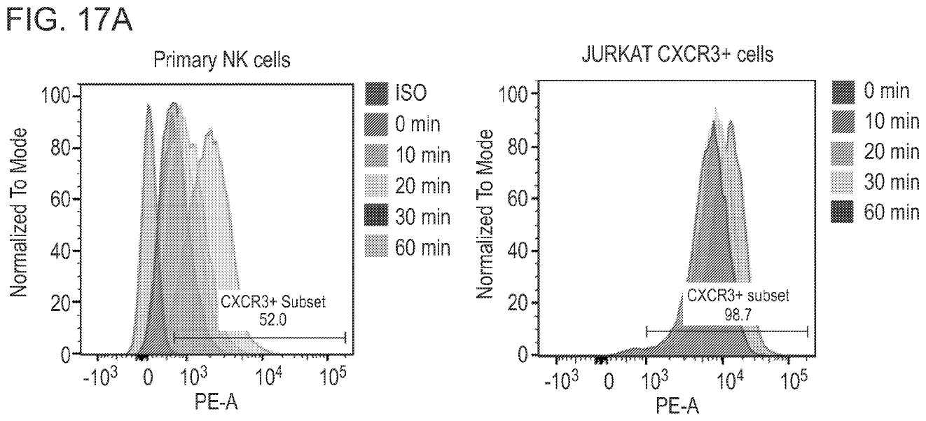

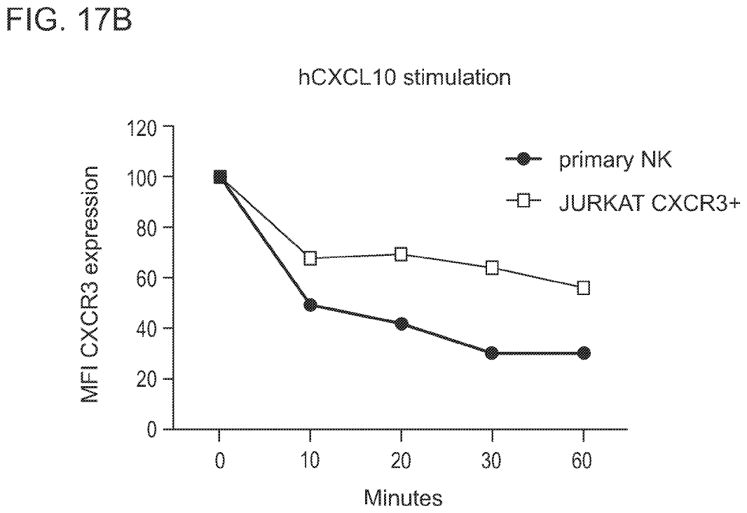

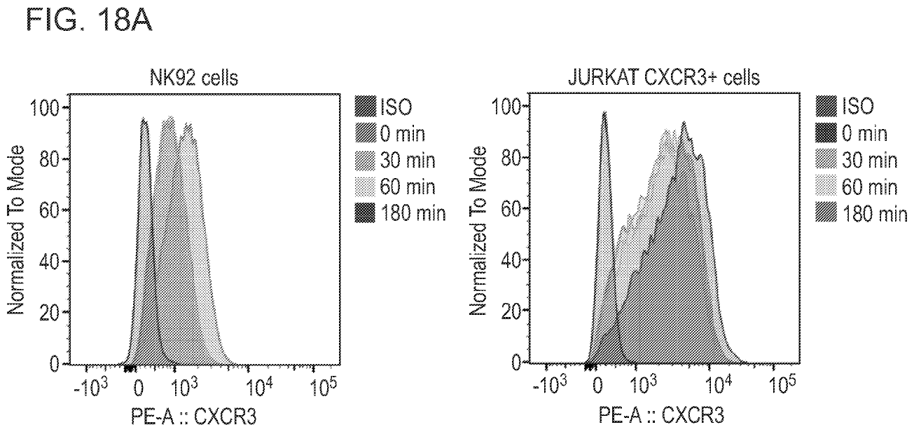

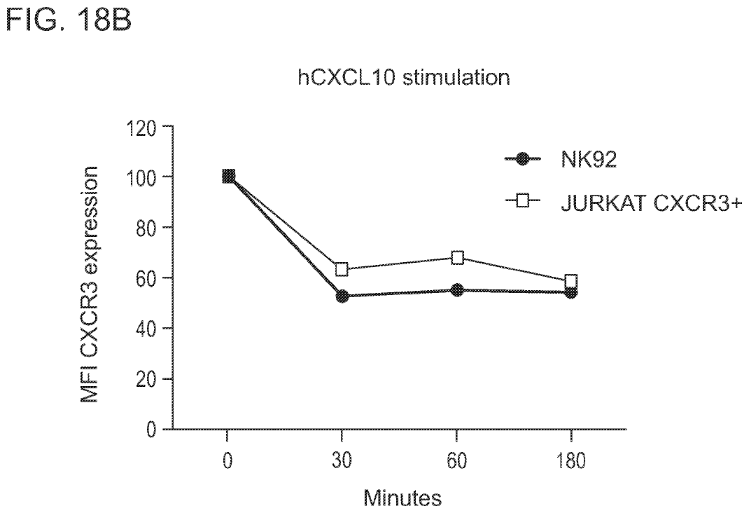

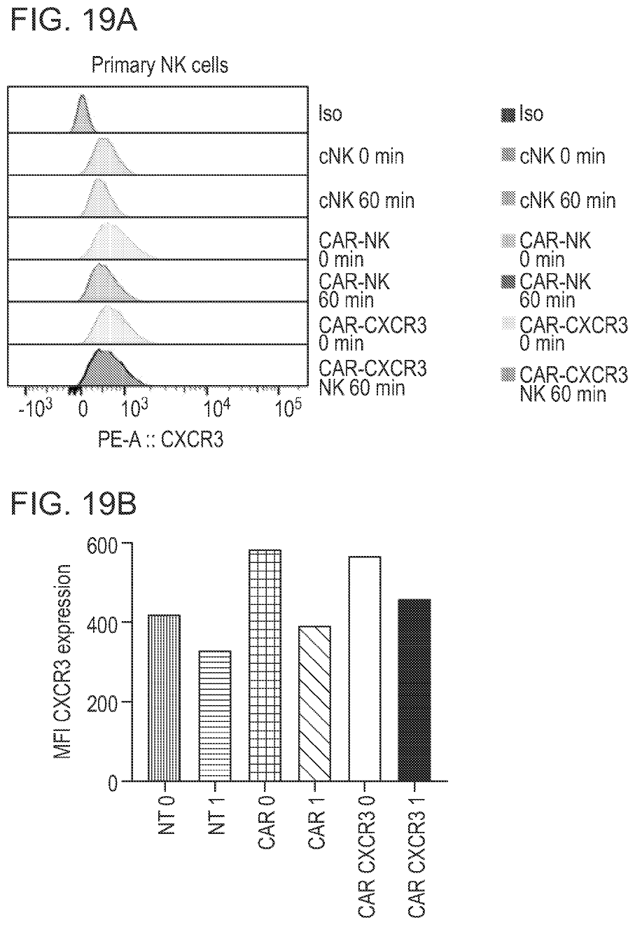

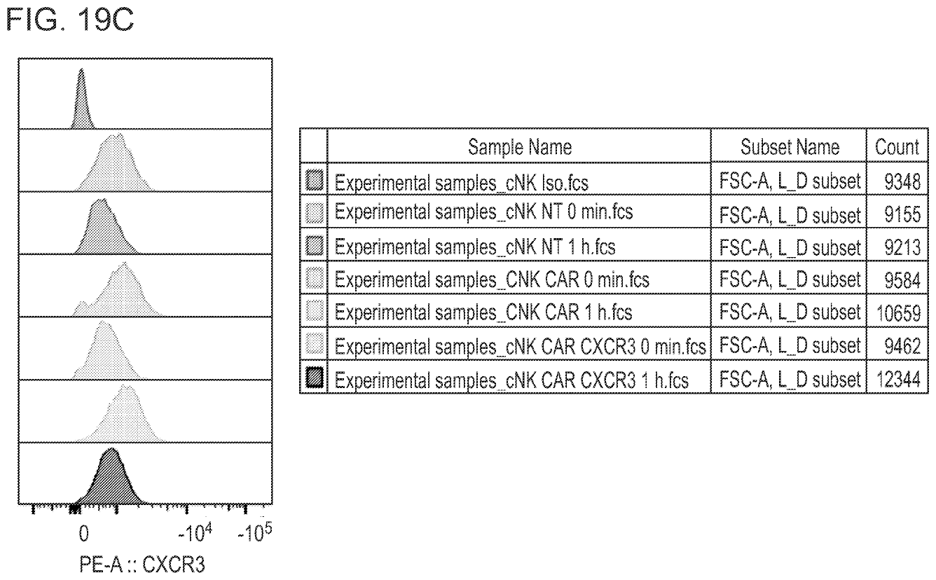

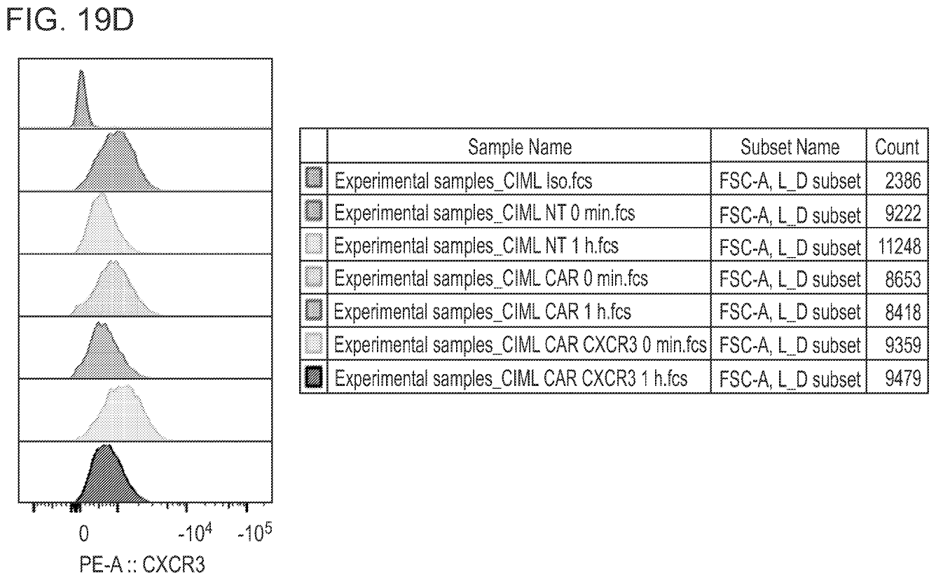

CXCR3 is degraded from the cell surface of primary NK cells and the NK cell lines NK92 and JURKAT both expressing CXCR3, after stimulation with 200 ng of recombinant human C—X—C Motif Chemokine Ligand 10 (hCXCL10) at different time points, as illustrated in FIG. 17A-FIG. 18B, measured by flow cytometry and expressed as median fluorescent intensity (MFI) of the CXCR3 receptor. CXCR3 is also degraded from CAR and CAR-CXCR expressing cNK cells. FIG. 18C-FIG. 18D illustrate CXCR3 surface expression as measured by flow cytometry of primary NK cells (cNK) expressing CAR, CAR-CXCR3 or control, stimulated with 200 ng of recombinant human CXCL10 stimulation at different time points (0 and 60 minutes). CXCR3 is degraded from cNK NT, CAR-cNK, and CAR-NK CXCR+ cell surfaces after 1 hour of hCXCL10 treatment (FIG. 19C). CXCR3 is also degraded from cytokine-induced memory-like (CIML) NK NT, CIML CAR-NK, CIML CAR-NK CXCR+ after 1 hour of hCXCL10 treatment (FIG. 19D). These data demonstrate that CXCR3 is lost from the cell surface after hCXCL10 treatment and that CXCR3-overexpressed cell lines (NK92, Jurkat) have a higher CXCR3 surface expression and consequently lower CXCR3 receptor internalization relative to endogenous NK CXCR3 expression.

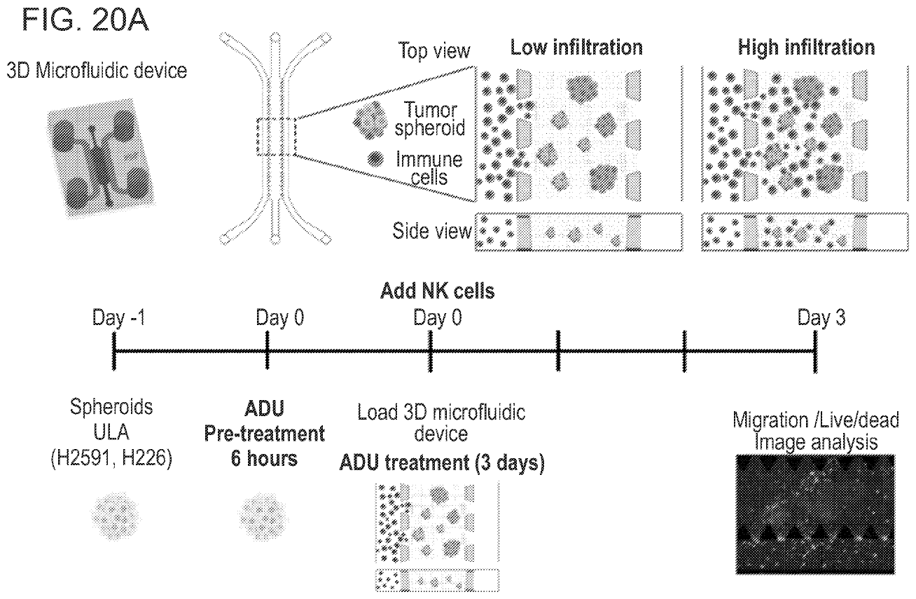

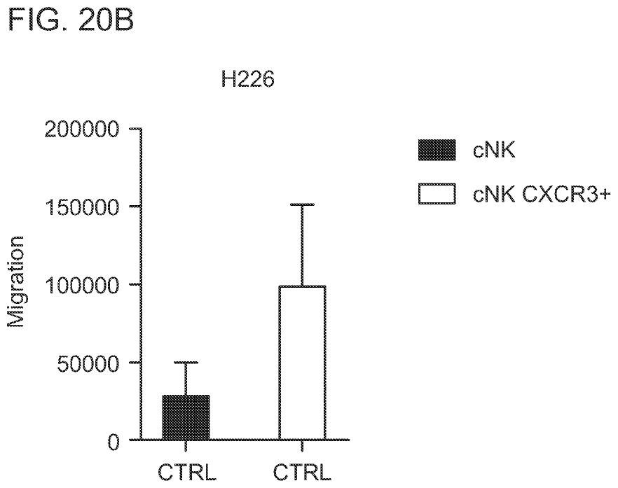

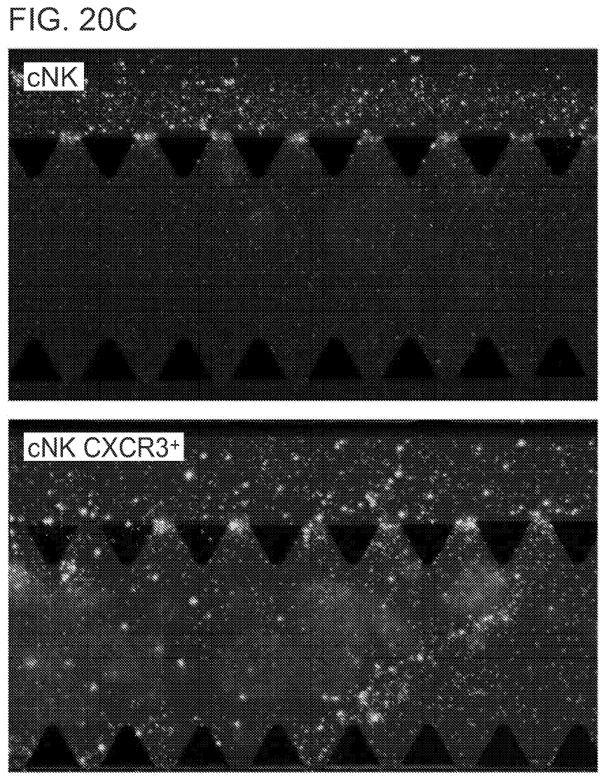

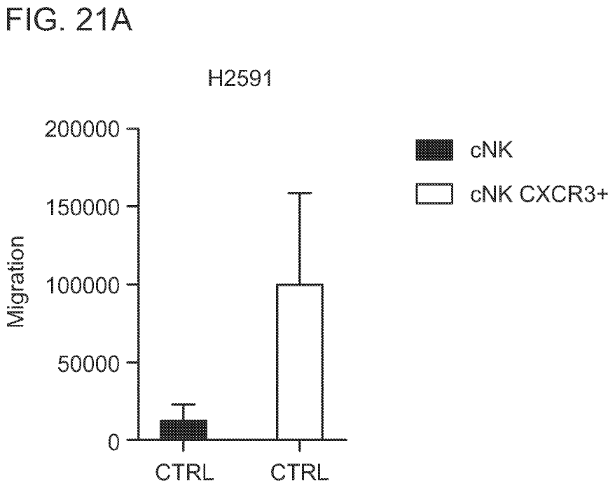

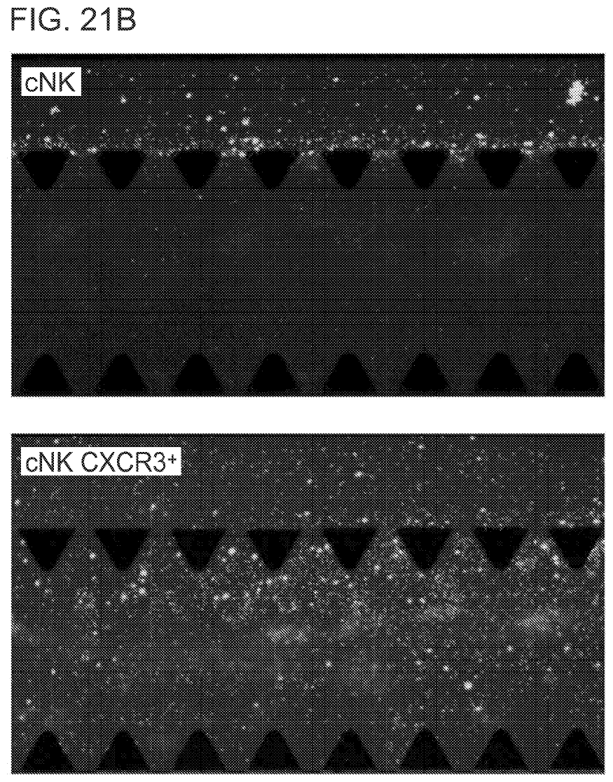

Immune cell migration assays were performed on control cNK cells and cNK cells overexpressing CXCR3. CXCR3 overexpression resulted in increased NK cell migration towards H226 MPM cells (FIG. 20A-FIG. 20B) and H2591 MPM cells (FIG. 21A-FIG. 21B).

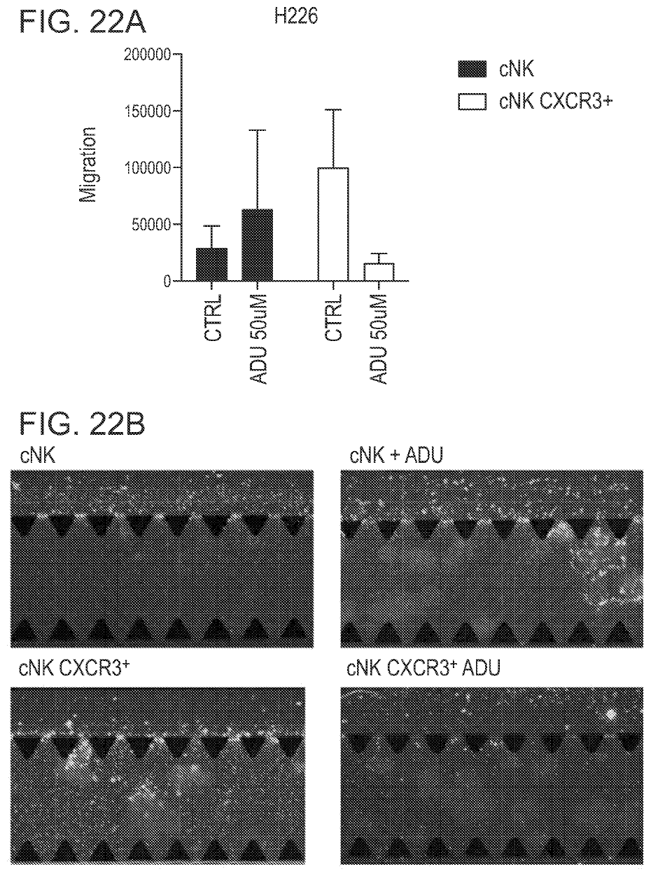

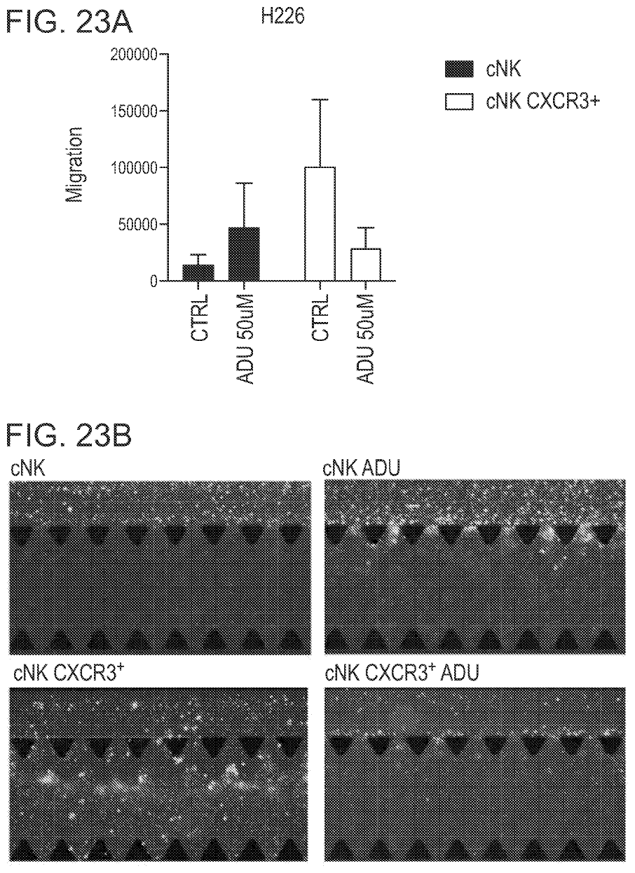

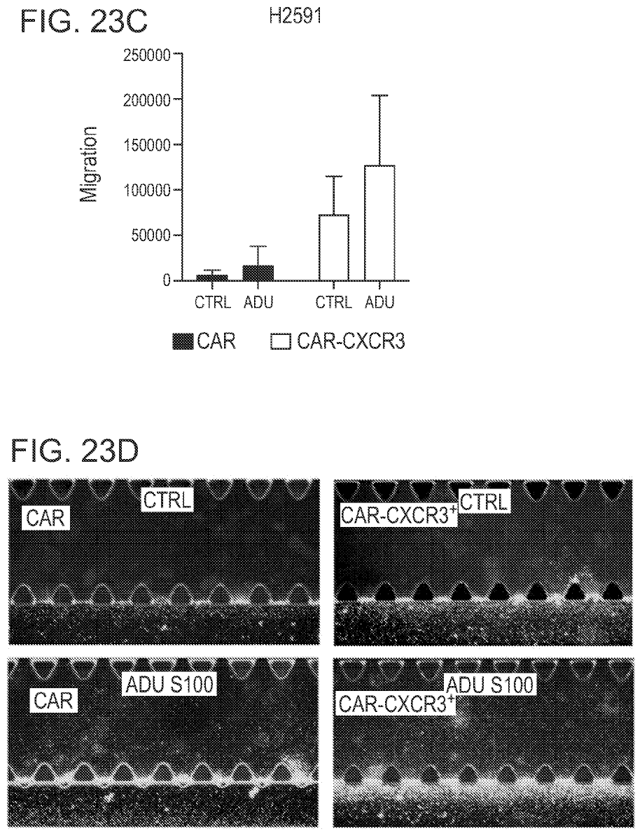

The effect of CXCR3 and ADU-S100 (abbreviated ADU) on NK cell migration was next tested. ADU-S100 increased migration of cNK cells, but decreased migration of cNK overexpressing CXCX3 towards H226 MPM cells (FIG. 22A-FIG. 22B) as well as towards H2591 MPM cells (FIG. 22A-FIG. 23B).

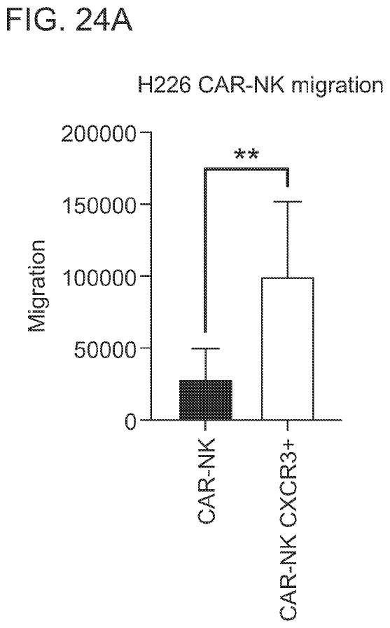

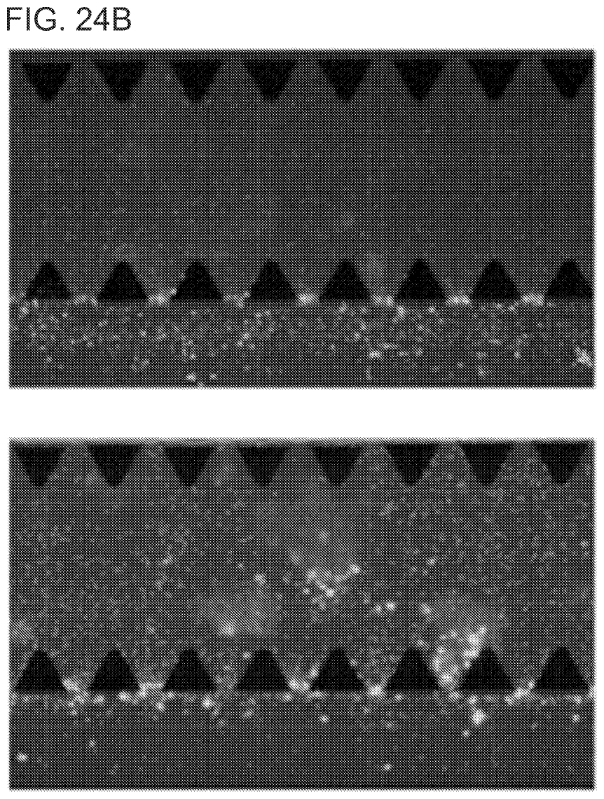

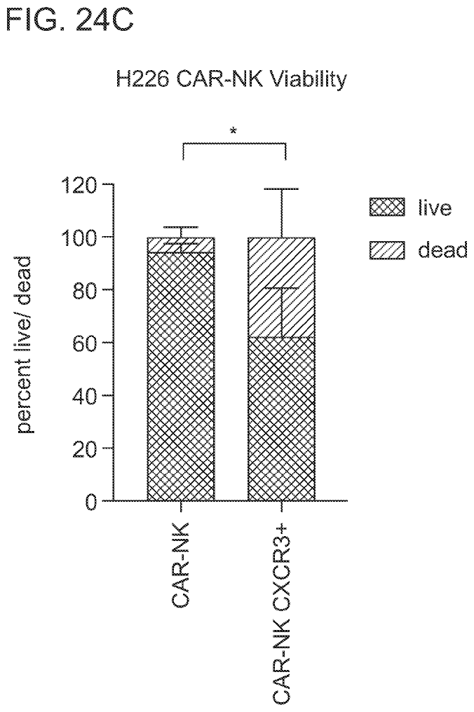

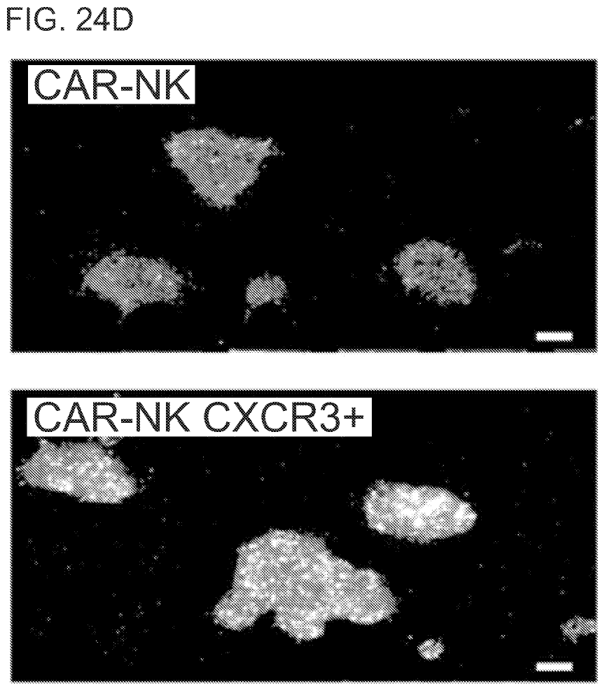

Furthermore, CXCR3 overexpression increases CAR-NK cell migration and cytotoxicity. CAR-NK control cells or CAR-NK cells overexpressing CXCR3 (CAR-NK CXCR+) were tested for migration towards H226 MPM cells and H226 cell killing. CAR-NK CXCR+ cells migrated (FIG. 24A-FIG. 24B) and killed more H226 cells than NK control cells (FIG. 24C-FIG. 24D). In FIG. 24D, cNK cells are labeled in red, all cells (live and dead) are labeled in blue with DAPI, and dead cells are labeled with Draq7 in yellow; the scale bar represents 150 μm.

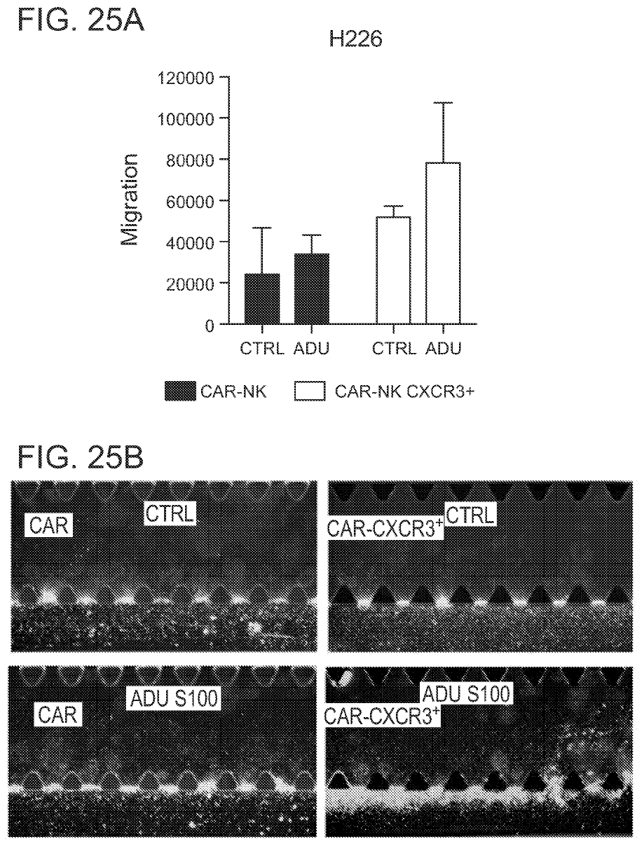

The STING agonist ADU-S100 enhances CAR-NK migration. CAR-NK control cells and CAR-NK CXCR+ cells were tested for migration with and without ADU-S100. ADU-S100 did not affect CAR-NK control cell migration towards H226 MPM cells; CAR-NK CXCR+ cell migration was increased after ADU-S100 treatment (FIG. 25A-FIG. 25B).

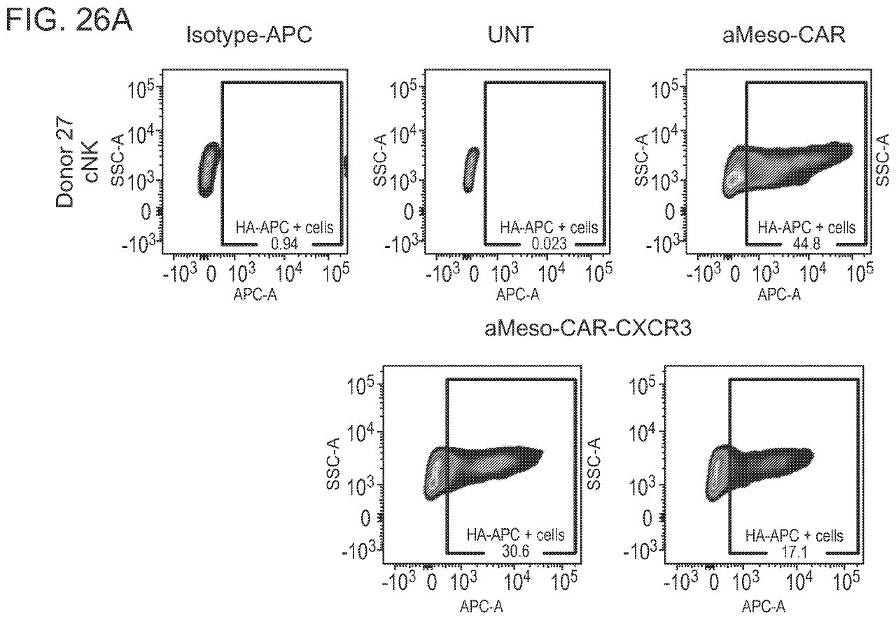

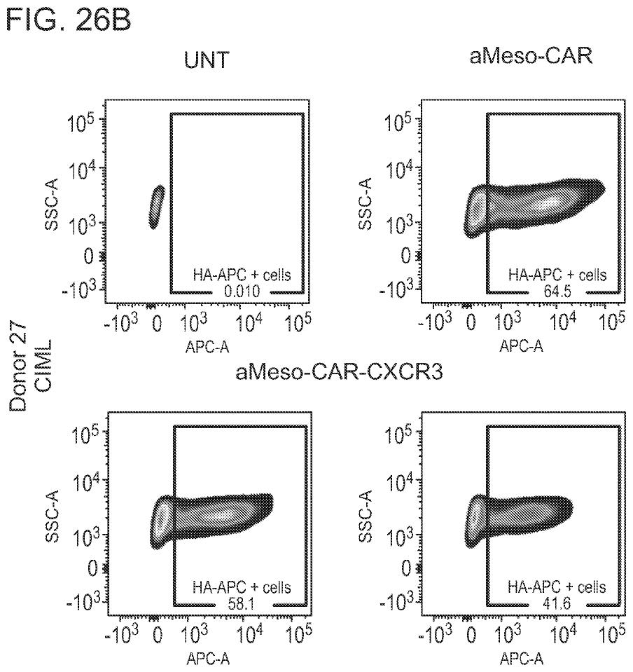

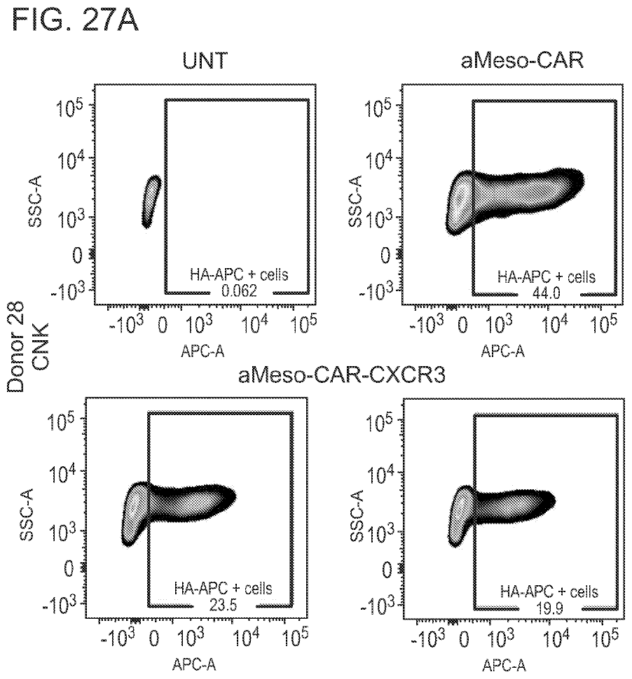

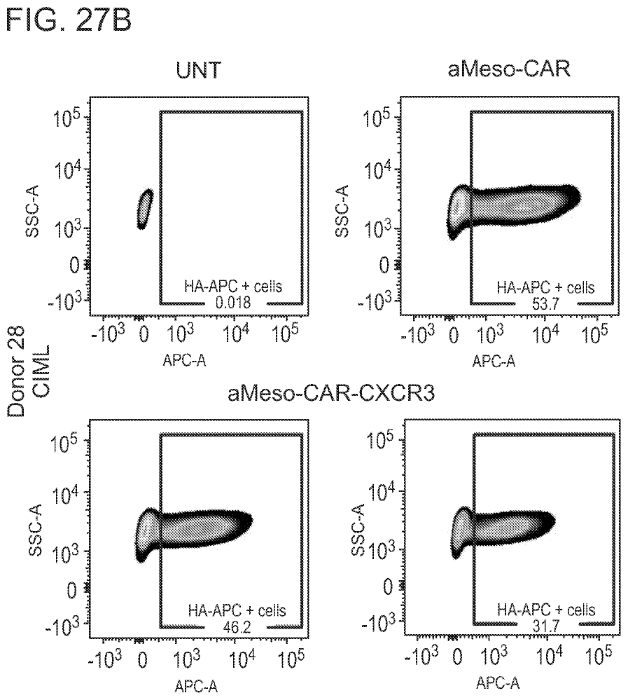

CAR expression was confirmed in cNK and CIML NK cells isolated and generated from two donors. Untransduced (abbreviated UNT) cNK and CIML NK cells did not show any binding against the anti-APC-HA antibody, while cNK and CIML NK cells transduced with an anti-mesothelin CAR construct (containing a human agglutinin (HA) tag; abbreviated CAR) with or without a CXCR3 overexpression construct had increased binding to the anti-APC-HA antibody (abbreviated CAR-CXCR3) (FIG. 26A-FIG. 27B). The fraction of CAR high cNK cells was 23.9±6.8% in CAR-CXCR expressing cells as compared to 44.8% in CAR only expressing cells and 0.02% in untransduced cells for donor 27 (FIG. 26A). The fraction of CAR high cNK cells was 21.7±1.8 in CAR-CXCR expressing cells as compared to 44% in CAR only expressing cells and 0.06% in untransduced cells for donor 28 (FIG. 27A). The fraction of CAR high CIML cells was 49.9±8.3% in CAR-CXCR expressing cells as compared to 64.5% in CAR only expressing cells and 0.01% in untransduced for donor 27 (FIG. 26B). The fraction of CAR high CIML cells was 39±7.3 in CAR-CXCR expressing cells as compared to 53.7 in CAR only expressing cells and 0.02% in untransduced for donor 28 (FIG. 27B).

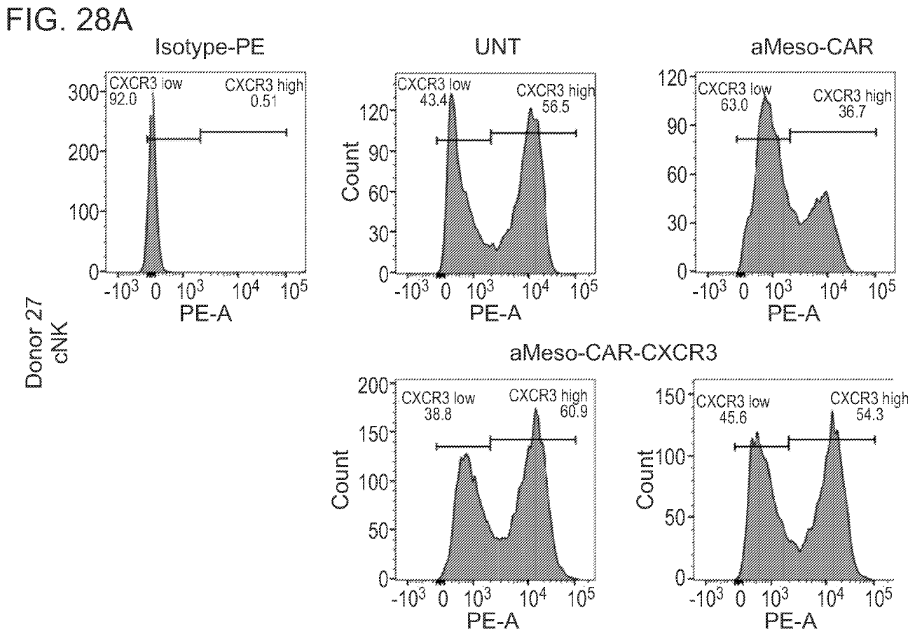

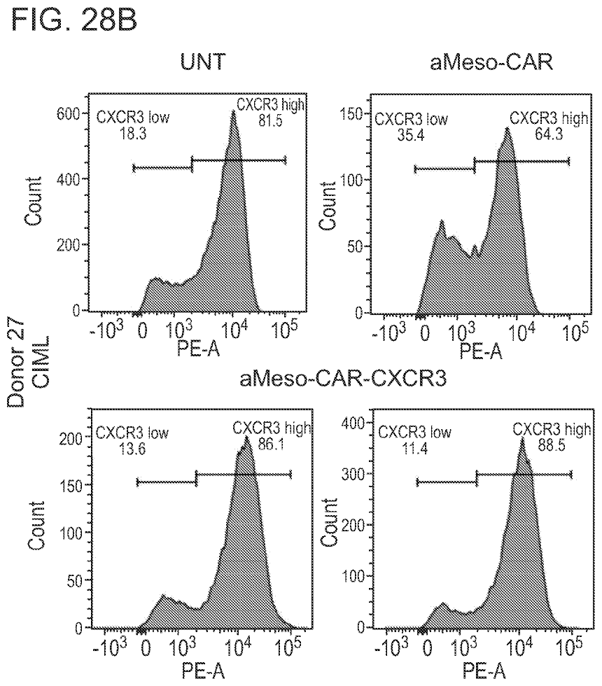

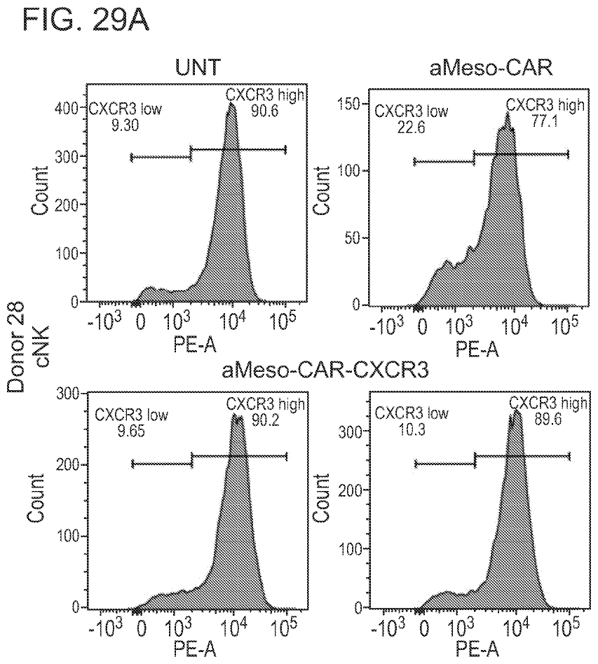

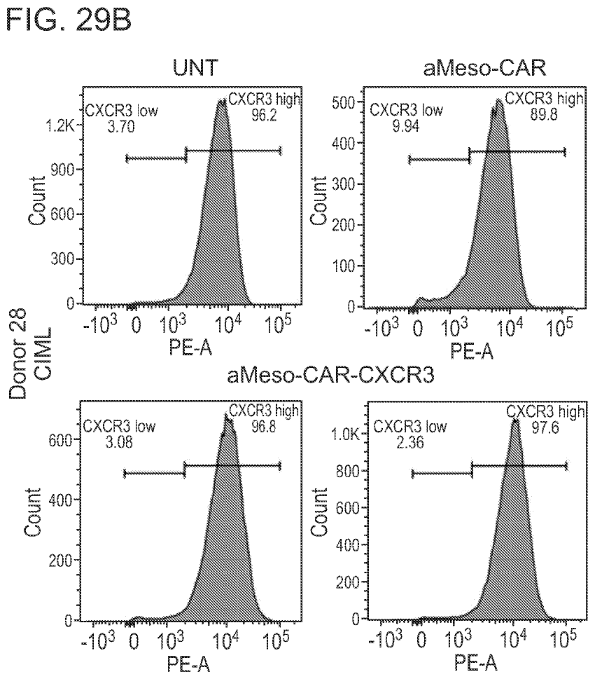

Similar to CAR expression confirmation, CXCR3 overexpression was confirmed by flow cytometry on cNK (FIG. 28A, FIG. 29A) and CIML NK cells (FIG. 28B, FIG. 29B) that were untransduced or transduced with an anti-mesothelin CAR construct with or without a CXCR3 overexpression construct (FIG. 28A-FIG. 29B). The fraction of CXCR3 high cNK cells increased to 57.6±3.3% in CAR-CXCR expressing cells as compared to 36.7% in CAR only expressing cells for donor 27 (FIG. 28A). The fraction of CXCR3 high cNK cells increased to 89.9±0.3 in CAR-CXCR expressing cells as compared to 77.1 in CAR only expressing cells for donor 28 (FIG. 29A). The fraction of CXCR3 high CIML cells increased to 87.3±1.2% in CAR-CXCR expressing cells as compared to 64.3% in CAR only expressing cells for donor 27 (FIG. 28B). The fraction of CXCR3 high CIML cells increased to 97.2±0.4 in CAR-CXCR expressing cells as compared to 89.8 in CAR only expressing cells for donor 28 (FIG. 29B).

More generally, combination immunotherapy remains challenging to translate to the clinic, and utilizing patient-derived tumor samples to study innate/adaptive immune crosstalk and the effects of activating one pathway on the broader TIME may inform the best approaches to enhance emerging cell therapies and overcome immune exhaustion.

All patent publications and non-patent publications are indicative of the level of skill of those skilled in the art to which this disclosure pertains. All these publications are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

What is claimed is:

1. A nucleic acid construct comprising:
a first nucleic acid comprising a first promotor operably linked to a nucleic acid encoding a C—X—C Motif Chemokine Receptor 3 (CXCR3); and
a second nucleic acid encoding a chimeric antigen receptor (CAR), wherein the CAR comprises a ligand binding domain comprising a single chain antibody fragment that binds an antigen on a tumor cell, a transmembrane domain, and an intracellular domain comprising a signaling domain.

2. The nucleic acid construct of claim 1, wherein the second nucleic acid is operably linked to a second promoter, which may be the same or different from the first promoter; or
wherein a third nucleic acid encoding a self-cleaving peptide is disposed between the first and second nucleic acids, and the first promoter drives expression of the first, the second, and the third nucleic acid.

3. The nucleic acid construct of claim 1, wherein the first promoter is configured to overexpress the CXCR3.

4. The nucleic acid construct of claim 1, wherein the antigen on the tumor cell is a malignant pleural mesothelioma (MPM) antigen.

5. The nucleic acid construct of claim 4, wherein the MPM antigen is mesothelin.

6. The nucleic acid construct of claim 5, wherein the ligand binding domain is derived from a portion of the anti-mesothelin ScFv YP218 antibody.

7. The nucleic acid construct of claim 1, wherein the signaling domain comprises a primary signaling domain, co-stimulatory signaling domain, or both a primary signaling domain and a co-stimulatory signaling domain.

8. The nucleic acid construct of claim 7, wherein the signaling domain comprises a CD3ζ primary signaling domain and a 4-1BB co-stimulatory signaling domain, or a CD28 co-stimulatory signaling domain, or both 4-1BB and CD28 co-stimulatory signaling domains.

9. A vector comprising the nucleic acid construct of claim 1.

10. The vector of claim 9, wherein the vector is a lentivirus vector.

11. The vector of claim 10, wherein the lentivirus vector is a baboon envelope pseudotyped lentiviral vector.

12. A genetically modified immune cell containing one or more vectors comprising a nucleic acid construct of claim 1.

13. The genetically modified immune cell of claim 12, wherein the genetically modified immune cell is a Natural Killer (NK) cell, a T cell, or a combination thereof.

14. A pharmaceutical composition comprising an effective number of the genetically modified immune cells of claim 12 and a pharmaceutically acceptable carrier.

15. A method of treating cancer in a subject, comprising:
administering to the subject in need thereof an effective amount of the pharmaceutical composition of claim 14.

16. The method of claim 15, further comprising administering to the subject an effective amount of a STING agonist prior to, substantially contemporaneous with, or subsequent to the administering of the pharmaceutical composition.

17. The method of claim 16, wherein the STING agonist comprises ADU-S100, TAK-676, BI-STING, BMS-986301, GSK532, JNJ-4412, MK-1454, SB11285, 3′3′-scylic AIMP, ALG-031048, E7766, JNJ-′6196, MK-2118, MSA-1, MSA-2, SNX281m SR-717, KAT676, TTI-10001, XMT-2056, CRD-5500, or a combination thereof.

18. The method of claim 17, wherein the STING agonist comprises ADU-S100 or TAK-676.

19. The method of claim 16, wherein the STING agonist is delivered by intratumoral or intravenous injection.

20. The method of claim 15, wherein the cancer is a solid tumor.

21. The method of claim 20, wherein the cancer is MPM, melanoma, gastric cancer, liver cancer, lung cancer, bladder cancer, colorectal cancer, or breast cancer.

22. The method of claim 21, wherein the cancer is MPM.
